(19) United States
(12) Patent Application Publication
(10) Pub. No.: US 2023/0263900 A1
(43) Pub. Date: Aug. 24, 2023

(54) DENDRIMER-DRUG CONJUGATE

(71) Applicant: Starpharma Pty Ltd., Abbotsford (AU)

(72) Inventors: David James Owen, Abbotsford (AU); Richard Hufton, Abbotsford (AU); Rosliana Halim, Abbortsford (AU)

(21) Appl. No.: 18/023,029

(22) PCT Filed: Aug. 31, 2021
(86) PCT No.: PCT/AU2021/051007

§ 371 Date: Feb. 24, 2023

(30) Foreign Application Priority Data
Aug. 31, 2020 (AU) 2020903111

(51) Int. Cl. A61K 47/60; A61K 47/64; A61K 31/675; A61K 31/706; A61P 31/14; A61K 9/00

(52) U.S. Cl. CPC A61K 47/60; A61K 47/645; A61K 31/675; A61K 31/706; A61P 31/14; A61K 9/0073; A61K 9/0019

(57) ABSTRACT

Provided herein are dendrimer-drug conjugates comprising a dendrimer including a core, building units which are lysine residues or analogues thereof, first terminal groups comprising a drug moiety comprising a Remdesivir nucleoside and a cleavable linker that provides for controlled release of the drug moiety, and second terminal groups comprising a hydrophobic polymeric group. Also provided herein are pharmaceutical compositions comprising the dendrimer-drug conjugates, and method and uses of the dendrimer-drug conjugates in therapy of disorders such as a viral infection, including a Coronavirus (CoV) infection.

### FIELD

The present disclosure relates to the delivery of antiviral agents by means of dendrimer-drug conjugates. The dendrimer-drug conjugates comprise a dendrimer including a core and building units, with the outermost generation of building units including one or more drug moieties comprising a Remdesivir nucleoside. The present disclosure also relates to pharmaceutical compositions and methods of treatment comprising the dendrimer-drug conjugates.

### BACKGROUND

A viral infection occurs when an organism's body is invaded by pathogenic viruses, and infectious virus particles (virions) attach to and enter susceptible cells. Viral diseases are usually detected upon clinical presentation, and symptoms can include, for example, severe muscle and joint pains preceding fever, skin rash, and swollen lymph glands. Viral infections are typically of limited duration, and so treatment usually entails reducing the associated symptoms.

The severity of the symptoms, and indeed patient outcome, is largely dependent on the type of viral infection. Coronaviruses (CoVs) are large, enveloped viruses with a positive sense, single-stranded RNA genome. CoV infections are a serious threat to both humans and animals; they cause enzootic infections and are responsible for outbreaks of severe acute respiratory syndrome (SARS) caused by SARS-CoV, Middle-East respiratory syndrome (MERS) caused by MERS-CoV and coronavirus disease 2019 (COVID-19) caused by SARS-CoV-2 in humans.

In humans, CoVs typically cause acute respiratory infections. Symptoms and severity can range from mild upper respiratory infections (e.g. a common cold) to much more severe acute respiratory distress syndrome (ARDS), pneumonia, to single and multi-organ failures. Part of human CoV virulence is attributed to long incubation periods and the display of no or often mild symptoms by infected persons, meaning that many infected patients do not realise they have been infected and continue their routines, thereby spreading infection.

Transmission of CoV is usually via airborne droplets to the nasal mucosa, where the virus then invades the respiratory tract. It is also possible that contaminated droplets on the hands may be transmitted to the oral and/or nasal mucosa. Currently, hygiene practices are recommended to prevent transmission and the disease is treated by symptom management. Mild symptoms, such as that of the common cold, are usually treated with nonsteroidal anti-inflammatory drugs.

While a substantial number of potential medications have been proposed based on previous work on SARS-CoV, and some initial clinical testing has taken place, currently, limited options for antiviral or immunomodulatory therapies for the prevention and/or treatment are available for use against SARS-CoV-2.

In particular, several candidates have emerged as potential new therapies in the treatment of SARS-CoV-2. Examples of such therapies that have been proposed include various kinase inhibitors (e.g., berzosertib, imatinib, baricitinib), angiotensin-II receptor blockers, cytokine-blocking monoclonal antibodies (e.g., anakinra, tocilizumab, sarilumab), antiretrovirals (e.g., lopinavir/ritonavir, emtricitabine/tenofovir), and other small molecule therapies (e.g., dexamethasone, colchicine, chloroquine/hydroxychloroquine, losartan, simvastatin).

Remdesivir, an antiviral medication developed by Gilead Sciences, has emerged as a further candidate antiviral therapy for the treatment of SARS-CoV-2. However, like many other medicines, Remdesivir has its drawbacks. In particular, Remdesivir is poorly soluble. This poor solubility means that prolonged administration times and high volume dosages are required to deliver the requisite dose of Remdesivir, which can present a significant burden for intensive care units (ICUs). Further, the poor solubility of Remdesivir requires it to typically be formulated with cyclodextrins. However, this in itself may be problematic due to possible side effects associated with the cyclodextrin component. Reported side-effects for the formulated Remdesivir product currently undergoing clinical trials include multiple-organ dysfunction syndrome, septic shock, acute kidney injury, low blood pressure and liver damage. In addition, the requisite daily intravenous delivery of Remdesivir places a burden on health care resources, which are under severe stress in a pandemic situation.

There remains a clear need for new therapies for the treatment of antiviral infections, and particularly SARS-CoV-2. There also remains a need for therapies that are safe, and/or which result in reduced side-effects experienced by already unwell patients. There also remains a need for therapies that are convenient and efficient to administer, therefore alleviating burden on ICUs and other SARS-CoV-2 treatment centres.

Any reference to any prior art in this specification is not, and should not be taken as an acknowledgement or any form of suggestion that the referenced prior art forms part of the common general knowledge.

### SUMMARY

It has now been found that dendrimer-drug conjugates containing a drug moiety comprising a Remdesivir nucleoside can be prepared which allow for controlled release of the free active agent from the dendrimer scaffold in vivo, and which provide for improved solubility of the drug moiety. It is considered that such conjugates provide a new therapy for the treatment of antiviral infections such as SARS-CoV-2, facilitate simple, less frequent dosing, and allow for therapeutic concentrations of active agent to be provided over a prolonged period of time.

Accordingly, in a first aspect, there is provided a dendrimer-drug conjugate comprising i) a core unit (C); and ii) building units (BU), each building unit being a lysine residue or an analogue thereof; wherein the core unit is covalently attached to at least two building units via amide linkages, each amide linkage being formed between a nitrogen atom present in the core unit and the carbon atom of an acyl group present in a building unit; and wherein the dendrimer-drug conjugate has from three to six generations of building units; and wherein building units of different generations are covalently attached to one another via amide linkages formed between a nitrogen atom present in one building unit and the carbon atom of an acyl group present in another building unit; the dendrimer-drug conjugate further comprising: iii) a plurality of first terminal groups (T1) attached to an outer building unit of the dendrimer, comprising a drug moiety comprising a Remdesivir nucleoside and a cleavable linker that provides for controlled release of the drug moiety; and iv) a plurality of second terminal groups (T2) attached to an outer building unit of the dendrimer, comprising a hydrophilic polymeric group; or a pharmaceutically acceptable salt thereof.

In some embodiments, the dendrimer-drug conjugate is capable of releasing in vivo:

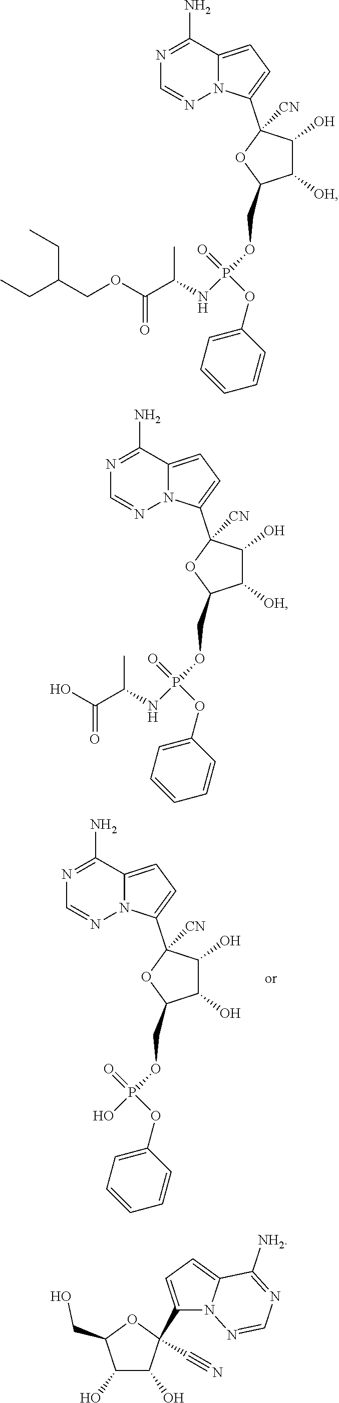

In some embodiments, the dendrimer-drug conjugate is capable of releasing in vivo:

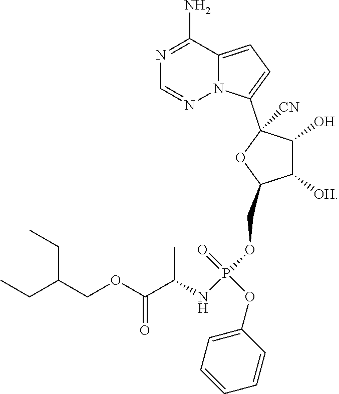

In some embodiments, the dendrimer-drug conjugate is capable of releasing in vivo:

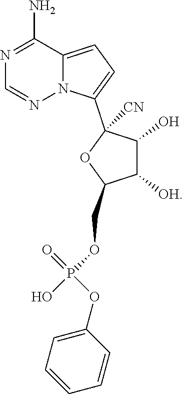

In some embodiments, the dendrimer-drug conjugate is capable of releasing in vivo:

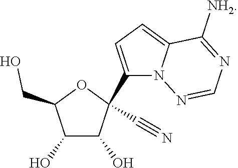 [SMILES: N#C[C@@]1(c2ccc3c(N)ncnn23)O[C@H](CO)[C@@H](O)[C@H]1O]

In some embodiments, the core unit is formed from a core unit precursor comprising two amino groups.

In some embodiments, the core unit is:

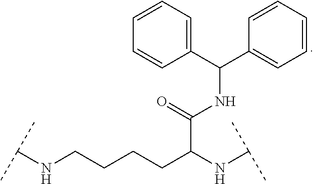

In some embodiments, the building units are each:

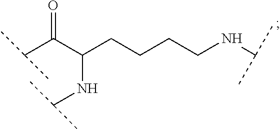

wherein the acyl group of each building unit provides a covalent attachment point for attachment to the core or to a previous generation building unit; and wherein each nitrogen atom provides a covalent attachment point for covalent attachment to a subsequent generation building unit, a first terminal group or a second terminal group.

In some embodiments, the building units are each:

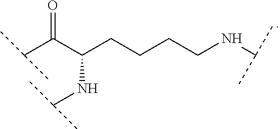

In some embodiments, the dendrimer has five generations of building units.

In some embodiments, the cleavable linker is covalently attached to the drug moiety such that, when exposed to PBS and 10% DMSO at pH 7.4 and 37° C., less than 50% of drug moiety is released from the conjugate within 24 hours.

In some embodiments, the cleavable linker is covalently attached to the drug moiety such that, when exposed to PBS and 10% DMSO at pH 7.4 and 37° C., within 5% to 40% of drug moiety is released from the conjugate within 24 hours.

In some embodiments, the cleavable linker is a diacyl linker group of formula:

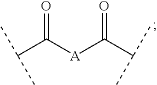

wherein A is a C₂-C₁₀ alkylene group which is optionally interrupted by at least one O, S, NH, or N(Me), or wherein A is a heterocycle selected from the group consisting of tetrahydrofuran, tetrahydrothiophene, pyrrolidine, and N-methylpyrrolidine.

In some embodiments, the cleavable linker is:

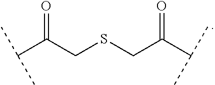

In some embodiments, the drug moiety is:

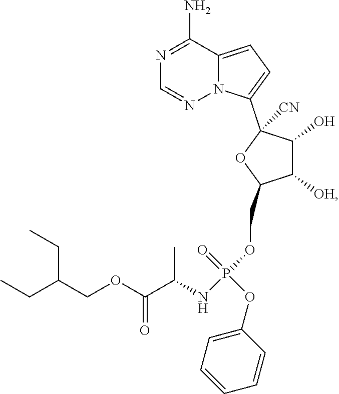

which is covalently attached to the cleavable linker through an —OH or —NH₂ group.

In some embodiments, the drug moiety is selected from the group consisting of:

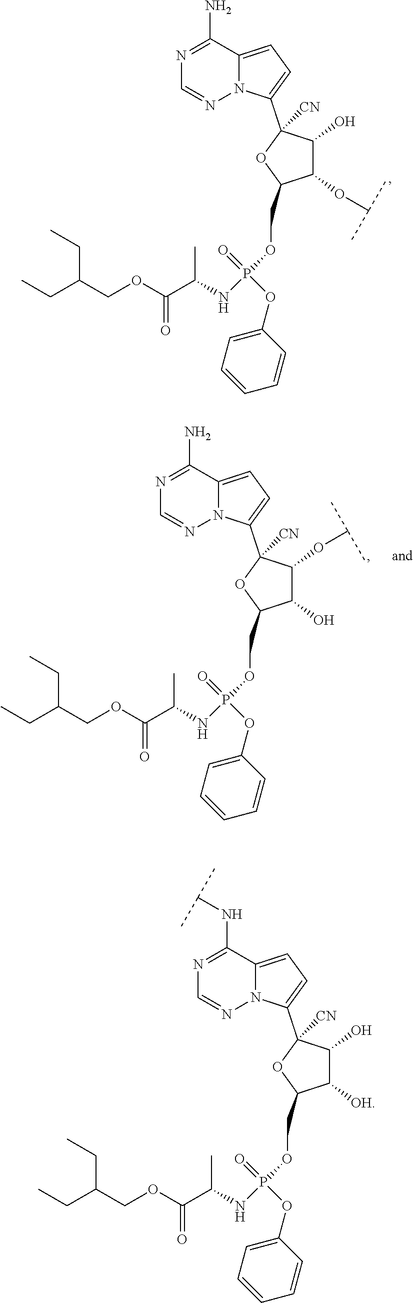

In some embodiments, the first terminal group is:

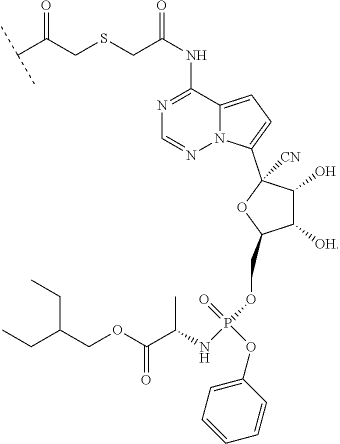

In some embodiments, the drug moiety is selected from the group consisting of:

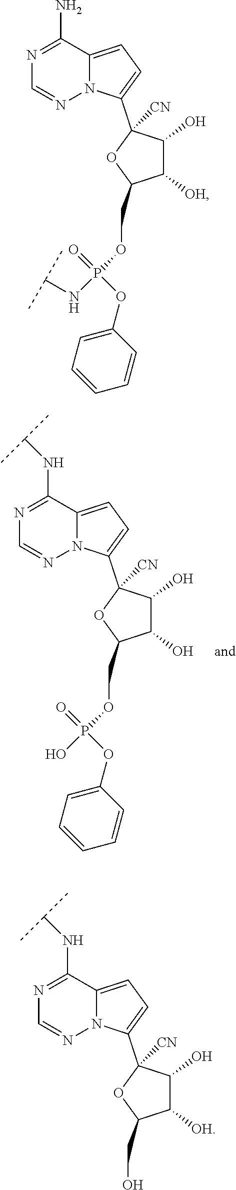

In some embodiments, the hydrophilic polymers comprise polyethylene gycol (PEG), polyethyloxazoline (PEOX) or polysarcosine groups.

In some embodiments, the second terminal groups comprise PEG groups having an average molecular weight in the range of from 500 to 2500 Daltons.

In some embodiments, the second terminal groups each comprise a PEG group covalently attached to a PEG linking group (L1) via an ether linkage formed between a carbon atom present in the PEG group and an oxygen atom present in the PEG linking group, and each second terminal group is covalently attached to a building unit via an amide linkage formed between a nitrogen atom present in a building unit and the carbon atom of an acyl group present in the PEG linking group.

In some embodiments, the second terminal group is:

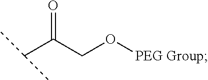

and wherein the PEG group is a methoxy-terminated PEG having an average molecular weight in the range of from 500 to 2500 Daltons.

In some embodiments, the dendrimer-drug conjugate comprises surface units comprising an outer building unit attached to a first terminal group and a second terminal group, the surface units having the structure:

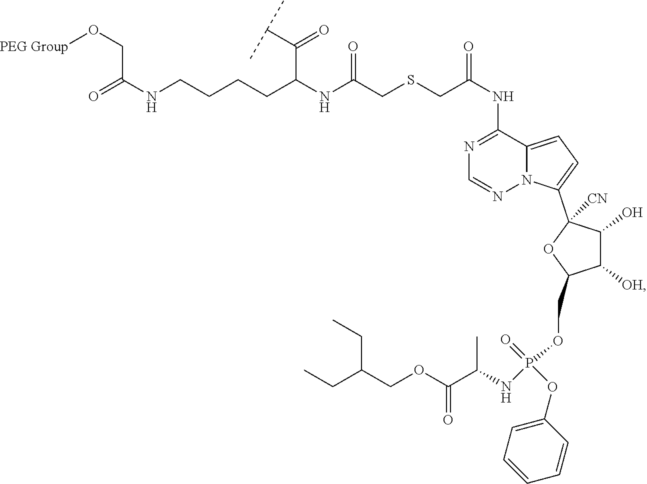

and wherein the PEG group is a methoxy-terminated PEG having an average molecular weight in the range of from 500 to 2500 Daltons.

In some embodiments, the dendrimer has five generations of building units, the five generations are complete generations, and wherein the outer generation of building units provides 64 nitrogen atoms for covalent attachment to a first terminal group or a second terminal group, wherein from 24 to 32 first terminal groups are covalently attached to one of said nitrogen atoms, and wherein from 24 to 32 second terminal groups are each covalently attached to one of said nitrogen atoms.

In some embodiments, the dendrimer-drug conjugate is:

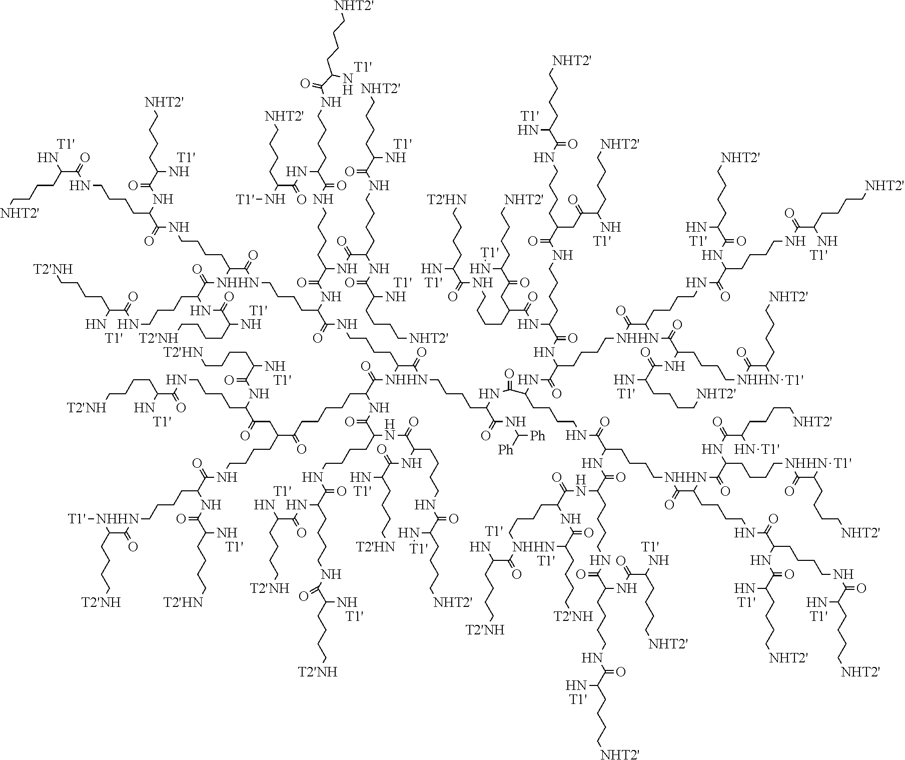

in which T1′ represents a group selected from the group consisting of hydrogen, and

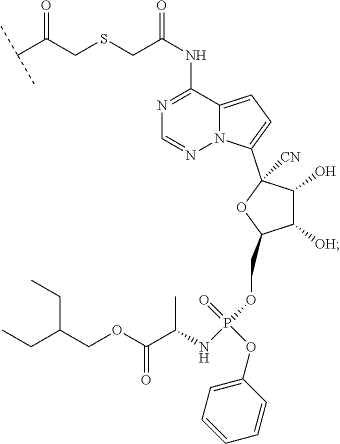

and wherein less than 10 of T1′ are hydrogen; and T2′ represents a second group which is

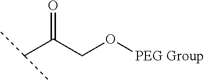

wherein the PEG group is a methoxy-terminated PEG having an average molecular weight in the range of from 500 to 2500 Daltons, or T2′ represents H, and wherein less than 10 of T2′ are H.

In a further aspect, there is provided a composition comprising a plurality of dendrimer-drug conjugates or pharmaceutically acceptable salts thereof, wherein the dendrimer-drug conjugates are as defined herein.

In a further aspect, there is provided a pharmaceutical composition comprising:
i) a dendrimer-drug conjugate as defined herein, or a pharmaceutically acceptable salt thereof; and
ii) a pharmaceutically acceptable excipient.

In some embodiments, the composition is free of cyclodextrin.

In some embodiments, the composition has greater aqueous solubility of drug moiety comprising Remdesivir nucleoside than Remdesivir, in terms of moles of Remdesivir nucleoside solubilised.

In some embodiments, the composition is a non-aqueous composition formulated for intramuscular injection.

In some embodiments, the composition is a solid composition formulated for pulmonary delivery.

In some embodiments, the composition is formulated for pulmonary delivery.

In some embodiments, the dendrimer-drug conjugate as defined herein, or pharmaceutical composition as defined herein, is for use in the treatment and/or prevention of a viral infection.

In a further aspect, there is provided a method of treating and/or preventing a viral infection comprising administering to a subject in need thereof a therapeutically effective amount of a dendrimer-drug conjugate as defined herein, or a pharmaceutical composition as defined herein.

In a further aspect, there is provided the use of a dendrimer-drug conjugate as defined herein, or of a composition as defined herein, in the manufacture of a medicament for the treatment and/or prevention of a viral infection.

In some embodiments, the viral infection is an RNA viral infection.

In some embodiments, the viral infection is a Coronavirus (CoV) infection.

In some embodiments, the Coronavirus (CoV) is selected from the group consisting of severe acute respiratory syndrome-related coronavirus-2 (SARS-CoV-2), human coronavirus OC43 (HCoV-OC43), human coronavirus HKU1 (HCoV-HKU1), human coronavirus 229E (HCoV-229E), human coronavirus NL63 (HCoV-NL63), severe acute respiratory-related coronavirus (SARS-CoV), and middle-east respiratory syndrome-related coronavirus (MERS-CoV), and subtypes or variants thereof.

In some embodiments, the Coronavirus (CoV) is SARS-CoV-2 or a subtype or variant thereof.

In some embodiments, the prevention and/or treatment of a viral infection includes preventing or reducing the likelihood or severity of a symptom associated with a Coronavirus (CoV) infection.

In some embodiments, the symptom associated with a Coronavirus (CoV) infection is one or more selected from the group consisting of fever, cough, sore throat, shortness of breath, viral shedding, respiratory insufficiency, runny nose, nasal congestion, bronchitis, headache, muscle pain, dyspnea, moderate pneumonia, severe pneumonia, and acute respiratory distress syndrome (ARDS).

In some embodiments, the dendrimer-drug conjugate or composition is administered parenterally.

In some embodiments, the dendrimer-drug conjugate or composition is administered intravenously.

In some embodiments, the dendrimer-drug conjugate or composition is administered by fast infusion or as a bolus.

In some embodiments, the dendrimer-drug conjugate or composition is administered intramuscularly.

In some embodiments, the dendrimer-drug conjugate or composition is administered subcutaneously.

In some embodiments, the dendrimer-drug conjugate or composition is administered by inhalation.

In some embodiments, a single dose of dendrimer-drug conjugate provides plasma levels of Remdesivir of greater than 10 ng/mL for at least 5 days.

In some embodiments, a single dose of dendrimer-drug conjugate provides plasma levels of Remdesivir of greater than 100 ng/mL for at least 2 days.

In some embodiments, a single dose of dendrimer-drug conjugate provides plasma levels of GS-441524 of greater than 10 ng/mL for at least 2 days.

In some embodiments, a single dose of dendrimer-drug conjugate provides plasma levels of GS-441524 of greater than 5 ng/mL for at least 5 days.

In some embodiments, a single dose of dendrimer-drug conjugate provides a therapeutically effective amount of Remdesivir nucleoside over a period of at least five days.

In some embodiments, a single dose of dendrimer-drug conjugate provides a therapeutically effective amount of the drug moiety comprising Remdesivir nucleoside over a period of at least two days.

In some embodiments, a single dose of dendrimer-drug conjugate provides therapeutic drug exposure (AUCinf) of at least 5000 ng/h/mL of Remdesivir.

In some embodiments, a single dose of dendrimer-drug conjugate provides therapeutic drug exposure (AUCinf) of at least 3000 ng/h/mL of GS-441524.

In some embodiments, the dendrimer is administered in combination with a further therapeutic agent used for therapy of a viral condition.

### BRIEF DESCRIPTION OF THE DRAWINGS

FIG. 1 shows synthetic schemes for synthesis of example dendrimer-drug conjugates.

FIG. 2 shows the solubility of BHALys [Lys]₂[Lys]₄[Lys]₈[Lys]₁₆[Lys]₃₂[(α-NH-3′ O-Glu-Remdesivir)₃₂(ε-NH-COPEG˜₂₀₀₀)₃₂] in water. A 200 mg/mL solution of BHALys [Lys]₂[Lys]₄[Lys]₈[Lys]₁₆[Lys]₃₂ (α-NH-3′ O-Glu-Remdesivir)₃₂(ε-NH-COPEG˜₂₀₀₀)₃₂] (vial on the right, 421-476-039-01) compared to a suspension of Remdesivir at 40 mg/mL.

FIG. 3 shows the extent to which Remdesivir RHa-1 is released from 6 constructs in PBS buffer (pH 7.4) at 37° C. over time for 6 constructs.

FIG. 4 shows plasma pharmacokinetics and biodistribution of D-DOX and biodistribution of a solution formulation of doxorubicin. Plasma concentration-time profile of a solution formulation of D-DOX at 5 mg/kg to rats by following a 3H-labelled scaffold or doxorubicin after intravenous administration (Panel A) or intratracheal (IT) instillation (Panel B). In panels A and B, the dose equivalents of doxorubicin given were scaled to 5 mg/kg to show how clearance of the dendrimer scaffold compares to clearance of the drug. This data demonstrates that only a small amount of doxorubicin is released into the systemic circulation following IT administration of the D-DOX dendrimer-drug conjugate in contrast to IV delivery. Biodistribution profiles of doxorubicin (0.6 mg dose) or D-DOX (1 mg dose) in BALF (Panel C) or lungs (Panel D) 1, 3 or 7 days after intratracheal instillation to rats show the clearance of an intratracheal dose from the lungs over time. This data demonstrates the retention of D-DOX in the lung tissue and BALF in contrast to free doxorubicin when delivered IT.

FIG. 5 shows the fold change in total flux (measured in radiance photons) emitted from the lungs of rats at 1 week and 2 weeks following pre-dose imaging.

FIG. 6 shows the organ biodistribution of 3H-labelled D-DOX 5 days after IV administration or 7 days after IT instillation of 5 mg/kg dendrimer to rats. Data are represented as mean±s.d. (n=3-7 rats). This further demonstrates the retention in the lung of D-DOX following IT administration in contrast to IV administration.

FIG. 7 shows the localization of PEGylated dendrimer in the lungs 30 mins (left panel) or 2 days (right panel) after IT instillation of a 100 μl dosing solution containing 1 mg dendrimer. This figure demonstrates that the dendrimer is distributed throughout the lung over time, and is retained, not expelled from the lung.

FIG. 8 shows the pharmacokinetics of a 68 kDa PEGylated dendrimer after aerosol administration to the lungs of rats at a dose of 1 mg. Data are represented as mean±s.d (n=4-5 rats). Panel A shows plasma concentration of dendrimer dosed IV (black symbols) or into the lungs using a microsprayer (white symbols). Panel B shows the biodistribution of the dendrimer 7 days after an IT dose.

FIG. 9 shows the pharmacokinetics of plasma Remdesivir for RHa-5, RHa-15, and Remdesivir administered IV or SC at a dose of 6.17 mg/kg.

FIGS. 10a and 10b show the pharmacokinetics of plasma GS-441524 for RHa-5, RHa-15, and Remdesivir administered IV or SC at a dose of 6.17 mg/kg.

### DESCRIPTION

### General Definitions

Unless specifically defined otherwise, all technical and scientific terms used herein shall be taken to have the same meaning as commonly understood by one of ordinary skill in the art (e.g., chemistry, biochemistry, medicinal chemistry, polymer chemistry, and the like).

As used herein, the term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−20%, more preferably +/−10%, of the designated value.

As used herein, the term “equivalent” refers to an amount that is “about” the same, as defined above.

As used herein, singular forms “a”, “an” and “the” include plural aspects, unless the context clearly indicates otherwise.

Throughout this specification, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “subject” refers to any organism susceptible to a disease or condition. In one embodiment, the disease or condition is cancer. For example, the subject can be a mammal, primate, livestock (e.g., sheep, cow, horse, pig), companion animal (e.g., dog, cat), or laboratory animal (e.g., mouse, rabbit, rat, guinea pig, hamster). In one example, the subject is a mammal. In one embodiment, the subject is human.

As used herein, the term “treating” includes alleviation of the symptoms associated with a specific disorder or condition and eliminating said symptoms. For example, as used herein, the phrase “treating a viral infection” refers to alleviating one or more symptoms, and/or duration of symptoms associated with a viral infection and reducing said symptoms. In one embodiment, the term “treating a viral condition” refers to a reduction in the severity of one or more of the symptoms associated with the viral infection and/or duration of symptoms. In one embodiment, the term “treating a viral condition” refers to elimination of one or more of the symptoms associated with the viral infection. In one embodiment, the term “treating a viral infection” refers to the elimination of the viral infection from the host.

As used herein, the term “prevention” includes prophylaxis of the specific disorder or condition. For example, as used herein, the term “preventing a viral infection” refers to the prevention of a subject contracting the viral infection. In one embodiment, the term “preventing a viral infection” refers to preventing the onset or duration of one or more symptoms associated with the viral infection.

As used herein, the terms “viral shedding” and “shedding”, and variants thereof, refer to the expulsion and release of virus progeny following successful reproduction during a host cell infection. The terms may refer to shedding of virus or viral material from bodies into the environment. It will be understood that a reduction in viral shedding, particularly viral shedding into the environment, may reduce transmission of Coronavirus (CoV) infection.

As would be understood by the person skilled in the art, a dendrimer-drug conjugate would be administered in a therapeutically effective amount. The term “therapeutically effective amount”, as used herein, refers to a dendrimer-drug conjugate being administered in an amount sufficient to alleviate or prevent to some extent one or more of the symptoms of the disorder or condition being treated. The result can be the reduction and/or alleviation of the signs, symptoms, or causes of a disease or condition, or any other desired alteration of a biological system. In one embodiment, the term “therapeutically effective amount” refers to a dendrimer-drug conjugate being administered in an amount sufficient to result in a reduction in the severity of one or more symptoms associated with a viral infection. In one embodiment, the term “therapeutically effective amount” refers to a dendrimer-drug conjugate being administered in an amount sufficient to result in the elimination of one or more of the symptoms associated with the viral infection. In one embodiment, the term “therapeutically effective amount” refers to a dendrimer-drug conjugate being administered in an amount sufficient to eliminate the viral infection from the host. In one embodiment, the term “therapeutically effective amount” refers to a dendrimer-drug conjugate being administered in an amount sufficient to prevent a subject contracting the viral infection. In one embodiment, the term “therapeutically effective amount” refers to a dendrimer-drug conjugate being administered in an amount sufficient to prevent the onset or reduce the duration of one or more symptoms associated with the viral infection.

The term, an “effective amount”, as used herein, refers to an amount of a dendrimer-drug conjugate effective to achieve a desired pharmacologic effect or therapeutic improvement without undue adverse side effects or to achieve a desired pharmacologic effect or therapeutic improvement with a reduced side effect profile.

Suitable salts of the dendrimer-drug conjugates include those formed with organic or inorganic acids or bases. As used herein, the phrase “pharmaceutically acceptable salt” refers to pharmaceutically acceptable organic or inorganic salts. Exemplary acid addition salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. Exemplary base addition salts include, but are not limited to, ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion. It will also be appreciated that non-pharmaceutically acceptable salts also fall within the scope of the present disclosure since these may be useful as intermediates in the preparation of pharmaceutically acceptable salts or may be useful during storage or transport.

Those skilled in the art of organic and/or medicinal chemistry will appreciate that many organic compounds can form complexes with solvents in which they are reacted or from which they are precipitated or crystallized. These complexes are known as “solvates”. For example, a complex with water is known as a “hydrate”. As used herein, the phrase “pharmaceutically acceptable solvate” or “solvate” refer to an association of one or more solvent molecules and a compound of the present disclosure. Examples of solvents that form pharmaceutically acceptable solvates include, but are not limited to, water, isopropanol, ethanol, methanol, DMSO, ethyl acetate, acetic acid, and ethanolamine.

As used herein, the term “dendrimer-drug conjugate” refers to a molecule containing a dendrimer covalently attached to a drug moiety.

As used herein, the term “dendrimer” refers to a molecule containing a core and dendrons attached to the core. Each dendron is made up of generations of branched building units resulting in a branched structure with increasing number of branches with each generation of building units. A “dendrimer”, and a “drug-dendrimer conjugate”, may include pharmaceutically acceptable salts or solvates as defined supra.

As used herein, the term “building unit” refers to a branched molecule which is a lysine residue or an analogue thereof having three functional groups, one for attachment to the core or a previous generation of building units and at least two functional groups for attachment to the next generation of building units or forming the surface of the dendrimer molecule.

As used herein, the term “attached” refers to a connection between chemical components by way of covalent bonding. The term “covalent bonding”, as used herein, refers to a chemical bond formed by the sharing of one or more electrons, especially pairs of electrons, between atoms. The term “covalent bonding” is used interchangeable with the term “covalent attachment”.

As used herein, the term “solubilisation excipient” refers to a formulation additive that is used to solubilise insoluble or sparingly soluble drugs into an aqueous formulation. Examples include surfactants such as polyethoxylated caster oils including Cremophor EL, Cremophor RH 40 and Cremophor RH 60, D-α-tocopherol-polyethylene-glycol 1000 succinate, polysorbate 20, polysorbate 80, solutol HS 15, sorbitan monoleate, poloxamer 407, Labrasol and the like.

### Dendrimer-Drug Conjugates

In a first aspect, there is provided a dendrimer-drug conjugate comprising a dendrimer conjugated to a drug moiety, the drug moiety comprising a Remdesivir nucleoside.

### Dendrimer

There is provided a dendrimer-drug conjugate comprising:
i) a core unit (C); and
ii) building units (BU), each building unit being a lysine residue or an analogue thereof;
   wherein the core unit is covalently attached to at least two building units via amide linkages, each amide linkage being formed between a nitrogen atom present in the core unit and the carbon atom of an acyl group present in a building unit; and wherein the dendrimer-drug conjugate has from three to six generations of building units; and
   wherein building units of different generations are covalently attached to one another via amide linkages formed between a nitrogen atom present in one building unit and the carbon atom of an acyl group present in another building unit;
the dendrimer-drug conjugate further comprising:
iii) a plurality of first terminal groups (T1) attached to an outer building unit of the dendrimer, comprising a drug moiety comprising a Remdesivir nucleoside and a cleavable linker that provides for controlled release of the drug moiety; and
iv) a plurality of second terminal groups (T2) attached to an outer building unit of the dendrimer, comprising a hydrophilic polymeric group;
or a pharmaceutically acceptable salt thereof.

### Core Unit

The core unit (C) of the dendrimer-drug conjugate is covalently attached to two building units via amide linkages, each amide linkage being formed between a nitrogen atom present in the core unit and the carbon atom of an acyl group present in a building unit. Accordingly, the core unit may for example be formed from a core unit precursor comprising two amino groups. Any suitable diamino-containing molecule may be used as the core unit precursor. In some embodiments, the core unit is:

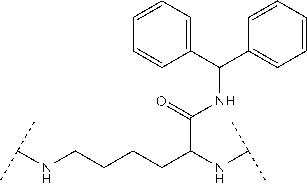

and may, for example, be formed from a core unit precursor:

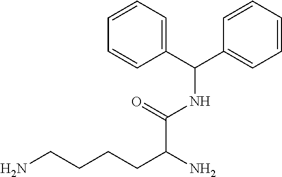 [SMILES: NCCCCC(N)C(=O)NC(c1ccccc1)c1ccccc1]

having two reactive (amino) nitrogens.

### Building Units

The building units (BU) are lysine residues or analogues thereof, and may be formed from suitable building unit precursors, e.g. lysine or lysine analogues containing appropriate protecting groups. Lysine analogues have two amino nitrogen atoms for bonding to a subsequent generation of building units and an acyl group for bonding to a previous generation of building units or a core. Examples of suitable building units include

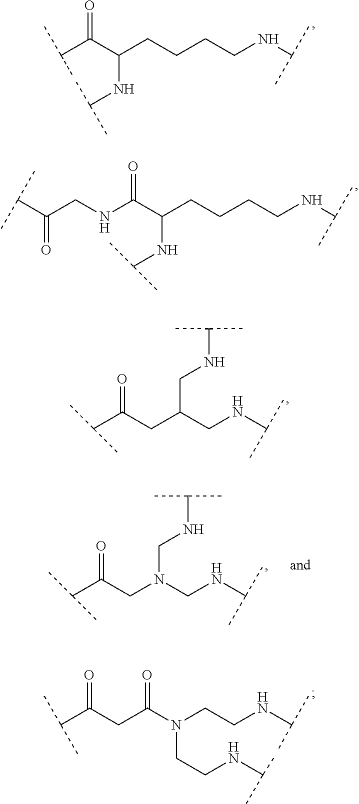

wherein the acyl group of each building unit provides a covalent attachment point for attachment to the core or to a previous generation building unit; and wherein each nitrogen atom provides a covalent attachment point for covalent attachment to a subsequent generation building unit, a first terminal group or a second terminal group.

In some preferred embodiments, the building units are each:

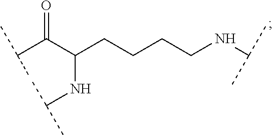

wherein the acyl group of each building unit provides a covalent attachment point for attachment to the core or to a previous generation building unit; and wherein each nitrogen atom provides a covalent attachment point for covalent attachment to a subsequent generation building unit, a first terminal group or a second terminal group.

In some preferred embodiments, the building units are each:

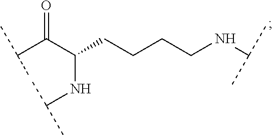

wherein the acyl group of each building unit provides a covalent attachment point for attachment to the core or to a previous generation building unit; and wherein each nitrogen atom provides a covalent attachment point for covalent attachment to a subsequent generation building unit, a first terminal group or a second terminal group.

The outermost generation of building units (BUₒᵤₜₑᵣ) may be formed by lysine or lysine analogue building units as used in the other generations of building units (BU) as described above. The outermost generation of building units (BUₒᵤₜₑᵣ) is the generation of building units that is outermost from the core of the dendrimer-drug conjugate, i.e., no further generations of building units are attached to the outermost generation of building units (BUₒᵤₜₑᵣ). These are also described as surface units or surface building units

It will be appreciated that the dendrons of the dendrimer-drug conjugate may, for example, be synthesised to the required number of generations through the attachment of building units (BU) accordingly. In some embodiments, each generation of building units (BU) may be formed of the same building unit, for example all of the generations of building units may be lysine building units. In some other embodiments, one or more generations of building units may be formed of different building units to other generations of building units.

The dendrimer-drug conjugate may have three, four, five or six generations of building units. In one embodiment, the dendrimer-drug conjugate is a three generation (G3) building unit dendrimer. In one embodiment, the dendrimer-drug conjugate is a four generation (G4) building unit dendrimer. In one embodiment, the dendrimer-drug conjugate is a five generation (G5) building unit dendrimer. In one embodiment, the dendrimer-drug conjugate is a six generation (G6) building unit dendrimer. In one example, where the dendrimer-drug conjugate is a five generation building unit dendrimer-drug conjugate, the structure of the dendrimer-drug conjugate includes five building units that are covalently linked to another, for example in the case where the building units are lysines, it may comprise the substructure:

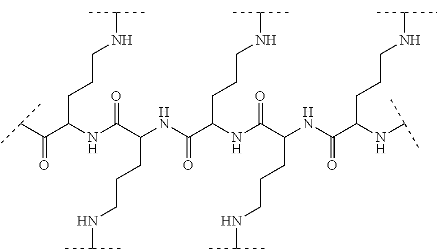

In some embodiments, the dendrimer has three complete generations of building units. With a core having two reactive amine groups, such a dendrimer will comprise 14 building units (i.e. core unit+2 BU+4 BU+8 BU). In some embodiments, the dendrimer has four complete generations of building units. With a core having two reactive amine groups, such a dendrimer will comprise 30 building units (i.e. core unit+2 BU+4 BU+8 BU+16 BU). In some embodiments, the dendrimer has five complete generations of building units. With a core having two reactive amine groups, such a dendrimer will comprise 62 building units (i.e. core unit+2 BU+4 BU+8 BU+16 BU+32 BU).

However, it will be appreciated that, due to the nature of the synthetic process for producing the dendrimer-drug conjugates, one or more reactions carried out to produce the dendrimer may not go fully to completion. Accordingly, in some embodiments, the dendrimer may comprise an incomplete generations of building units. For example, a population of dendrimer-drug conjugates may be obtained, in which the dendrimer-drug conjugates have a distribution of numbers of building units per dendrimer.

In some embodiments, when the dendrimer has three generations of building units, a population of dendrimer-drug conjugates is obtained which has a mean number of building units per dendrimer of at least 11, or at least 12, or at least 13. In some embodiments, when the dendrimer has three generations of building units, a population of dendrimer-drug conjugates is obtained in which at least 60%, at least 70%, at least 80%, at least 90% or at least 95% of the dendrimers have 11 or more building units. In some embodiments, when the dendrimer has three generations of building units, a population of dendrimer-drug conjugates is obtained in which at least 60%, at least 70%, at least 80%, at least 90% or at least 95% of the dendrimers have 13 or more building units. In some embodiments, when the dendrimer has four generations of building units, a population of dendrimer-drug conjugates is obtained which has a mean number of building units per dendrimer of at least 26, or at least 27, or at least 28, or at least 29. In some embodiments, when the dendrimer has four generations of building units, a population of dendrimer-drug conjugates is obtained in which at least 60%, at least 70%, at least 80%, at least 90% or at least 95% of the dendrimers have 26 or more building units. In some embodiments, when the dendrimer has four generations of building units, a population of dendrimer-drug conjugates is obtained in which at least 60%, at least 70%, at least 80%, at least 90% or at least 95% of the dendrimers have 29 or more building units. In some embodiments, when the dendrimer has five generations of building units, a population of dendrimer-drug conjugates is obtained which has a mean number of building units per dendrimer of at least 55, or at least 56, or at least 57, or at least 58, or at least 59, or at least 60. In some embodiments, when the dendrimer has five generations of building units, a population of dendrimer-drug conjugates is obtained in which at least 60%, at least 70%, at least 80%, at least 90% or at least 95% of the dendrimers have 55 or more building units. In some embodiments, when the dendrimer has five generations of building units, a population of dendrimer-drug conjugates is obtained in which at least 60%, at least 70%, at least 80%, at least 90% or at least 95% of the dendrimers have 60 or more building units.

Each reactive (amino) group of the core represents a conjugation site for a dendron comprising one or more generations of building units. The core has two reactive (amino) groups, and two dendrons, for the generations of building units to be attached.

In some embodiments, each generation of building units in each dendron (X) may be represented by the formula [BU]₂(b-1), wherein b is the generation number. As an example, a dendron (X) having five complete generations of building units is represented as [BU]₁-[BU]₂-[BU]₄-[BU]₈-[BU]₁₆.

### Drug Moiety

The dendrimer-drug conjugates comprise a plurality of first terminal groups (T1) comprising a drug moiety comprising a Remdesivir nucleoside.

Remdesivir is a therapeutic agent originally developed by Gilead Sciences (USA) for the treatment of Ebola virus disease and Marburg virus infections. Accordingly, Remdesivir finds application in viral therapy. Remdesivir is a pro-drug that can be metabolised, including into GS-441524. The structures of Remdesivir and GS-441524 are:

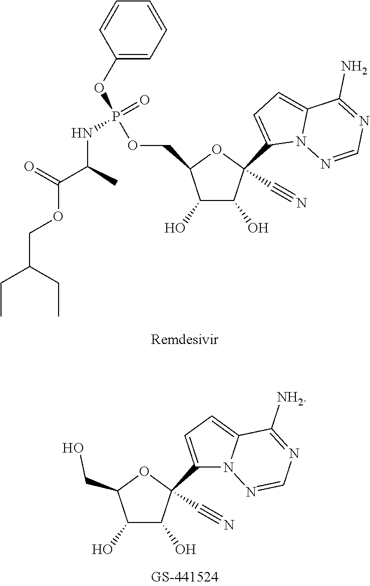 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1 ; N#C[C@@]1(c2ccc3c(N)ncnn23)O[C@H](CO)[C@@H](O)[C@H]1O]

Remdesivir and related compounds, and synthetic procedures for producing Remdesivir are described in, for example, WO2016/069825A1 and WO2012/012776 A1, the entire contents of which are incorporated herein in their entirety.

The chemical name for Remdesivir is 2-Ethylbutyl (2S)-2-{[(S)-{[(2R,3S,4R,5R)-5-(4-aminopyrrolo[2,1-f][1,2,4]triazin-7-yl)-5-cyano-3,4-dihydroxytetrahydrofuran-2-yl]methoxy}(phenoxy)phosphoryl]amino}propanoate. The molecular formula is C27H35N6O8P and the molecular weight is 602.6 g/mol.

Remdesivir inhibits viral RNA polymerases and has broad spectrum activity against members of the filoviruses (eg, EBOV, MARV), CoVs (eg, SARS-CoV, MERS-CoV), and paramyxoviruses (eg, respiratory syncytial virus [RSV], Nipah virus [NiV], and Hendra virus).

Remdesivir (GS-5734) is a single diastereomer monophosphoramidate prodrug of a monophosphate nucleoside analog (GS-441524).

On IV administration, rapid decline in plasma levels of Remdesivir is accompanied by the sequential appearance of the intermediate metabolite GS-704277 and the nucleoside metabolite GS-441524. GS-441524 is understood to be phosphorylated to the monophosphate which undergoes rapid conversion to the pharmacologically active analog of adenosine triphosphate (GS-443902) that inhibits viral RNA polymerases.

The drug moiety contains one or more hydroxyl or amine groups that can be utilised for linking of the drug moiety to the remainder of the dendrimer, via a linker, to form the dendrimer-drug conjugate.

The drug moiety comprises a Remdesivir nucleoside, i.e. it comprises the nucleobase and sugar component present in Remdesivir. In some embodiments, the drug moiety may contain additional structural components present in Remdesivir. For example, in some embodiments the drug moiety comprises a Remdesivir nucleotide, i.e. it contains a phosphorous-containing group. In some embodiments, the drug moiety comprises the entire structure of Remdesivir.

Accordingly, in one embodiment, the drug moiety is:

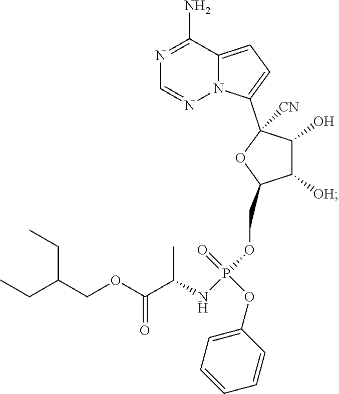

and it is this drug moiety that can be released from the dendrimer of the dendrimer-drug conjugate.

In one embodiment, the drug moiety is:

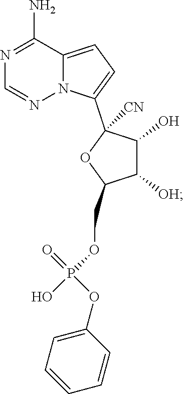

and it is this drug moiety that can be released from the dendrimer of the dendrimer-drug conjugate.

In one embodiment, the drug moiety is:

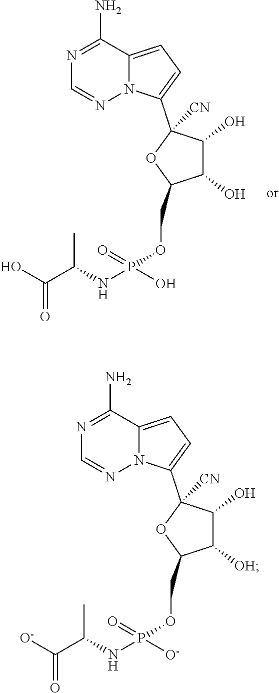

and it is this drug moiety that can be released from the dendrimer of the dendrimer-drug conjugate.

In one embodiment, the drug moiety is:

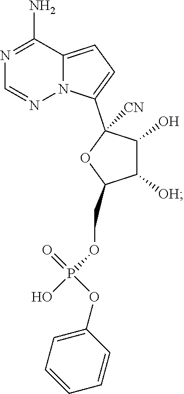

and it is this drug moiety that can be released from the dendrimer of the dendrimer-drug conjugate.

In one embodiment, the drug moiety is:

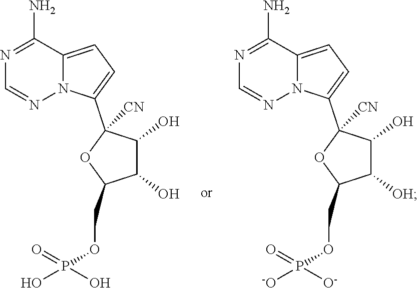

and it is this drug moiety that can be released from the dendrimer of the dendrimer-drug conjugate.

In one embodiment, the drug moiety is:

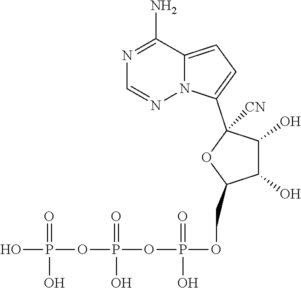 [SMILES: N#C[C@@]1(c2ccc3c(N)ncnn23)O[C@H](COP(=O)(O)OP(=O)(O)OP(=O)(O)O)[C@@H](O)[C@H]1O]

or a charged form thereof;
and it is this drug moiety that can be released from the dendrimer of the dendrimer-drug conjugate.

In one embodiment, the drug moiety is:

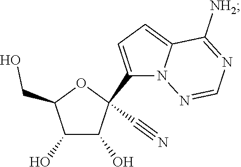 [SMILES: N#C[C@@]1(c2ccc3c(N)ncnn23)O[C@H](CO)[C@@H](O)[C@H]1O]

and it is this drug moiety that can be released from the dendrimer of the dendrimer-drug conjugate.

That is, upon in vivo administration, typically the dendrimer releases the drug moiety from the dendrimer-drug conjugate.

In some embodiments, the drug moiety is attached to the diacyl linker through an available —OH or —NH₂ group on the Remdesivir nucleoside. In one embodiment, the drug moiety is attached through a 3′-OH group, and the drug moiety is:

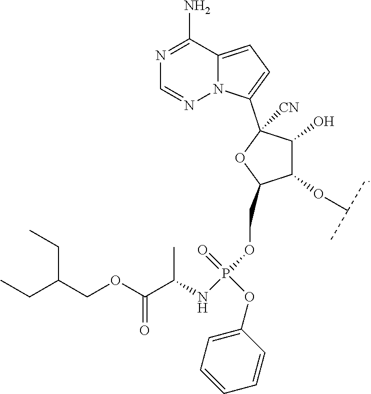

In one embodiment, the drug moiety is attached through a 2′-OH group, and the drug moiety is:

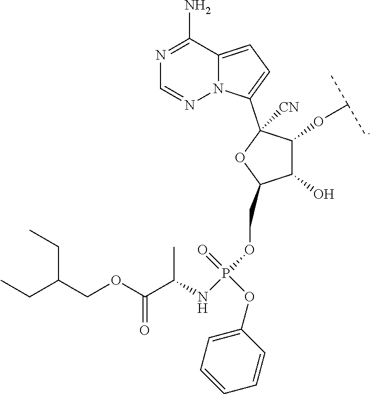

In one embodiment, the drug moiety is attached through an —NH₂ group, and the drug moiety is:

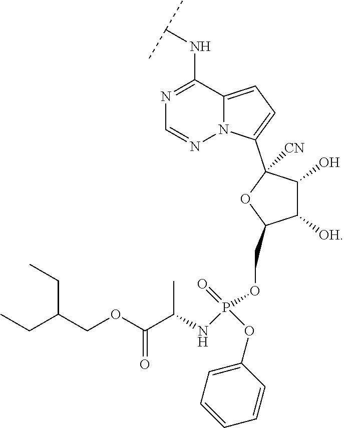

In some embodiments, the drug moiety attached to the dendrimer, and subsequently released from the dendrimer, may be a combination of any of

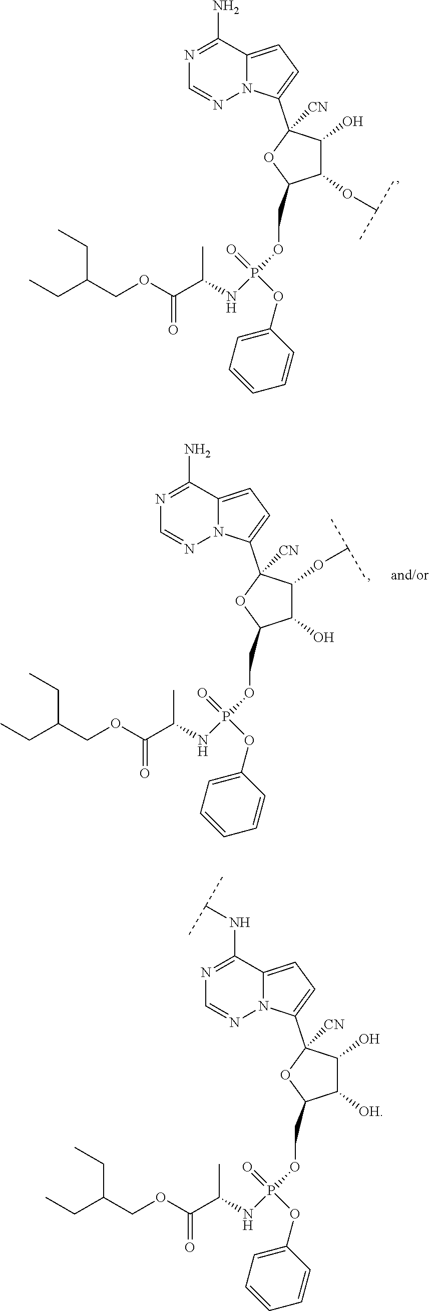

### Linker

The first terminal groups comprise a cleavable linker that provides for controlled release of the drug moiety. On in vivo administration, the linker is cleaved releasing drug moiety at a controlled rate so as to enable sustained provision of therapeutically effective concentrations of drug moiety over a period of time. The linker is selected so as to be cleavable at an appropriate rate in the body, for example in plasma, within cells, or within the lung or respiratory tract.

In some embodiments, the linker enables release of drug moiety from the drug-dendrimer conjugate upon exposure to aqueous media at pH 7.4 and 37° C. (e.g. PBS with 10% DMSO) at a rate of at least 5%, or at least 10% of dendrimer-bound drug moiety being released within 12 hrs, or at a rate of at least 5%, at least 10%, or at least 20% of dendrimer-bound drug moiety being released within 24 hours. In some embodiments, the linker enables release of drug moiety from the drug-dendrimer conjugate upon exposure to aqueous media at pH 7.4 and 37° C. (e.g. PBS with 10% DMSO) at a rate of not more than 50%, or not more than 40%, or not more than 30% of dendrimer-bound drug moiety being released within 12 hrs, or at a rate of not more than 60%, not more than 50%, or not more than 40%, or not more than 30% of dendrimer-bound drug moiety being released within 24 hours. In some embodiments the linker enables release of drug moiety from the drug-dendrimer conjugate upon exposure to aqueous media at pH 7.4 and 37° C. (e.g. PBS with 10% DMSO) at a rate in the range of from 5% to 90%, or from 5% to 80%, or from 5% to 50%, or from 5% to 40%, or from 5% to 30%, or from 10% to 50%, or from 10% to 40% or from 10% to 30% of dendrimer-bound drug moiety being released within 12 hours, or at a rate in the range of from 5% to 90%, from 5% to 80%, from 5% to 50%, from 5% to 40%, from 5% to 20%, from 5% to 15%, from 10% to 90%, from 10% to 80%, from 10% to 70%, 10% to 60%, from 10% to 50%, from 10% to 40%, from 10% to 30%, from 20% to 90%, from 20% to 80%, 20% to 70%, from 20% to 60%, from 20% to 50%, from 20% to 40%, or from 20% to 30%, or from 50% to 100%, from 60% to 90% of dendrimer-bound drug moiety being released within 24 hours.

In some embodiments, the linker enables release of drug moiety from the drug-dendrimer conjugate upon exposure to aqueous media at pH 7.4 and 37° C. (e.g. PBS with 10% DMSO) at a rate of less than 50%, or less than 40%, or less than 30% of dendrimer-bound drug moiety being released within 12 hrs, or at a rate of less than 60%, less than 50%, or less than 40%, or less than 30% of dendrimer-bound drug moiety being released within 24 hours.

A variety of suitable linkers may be used, as described in WO2012167309 (2012). The first terminal group comprises a cleavable group. Examples of suitable cleavable groups include those containing ester groups, amide groups (i.e. labile amide groups), disulfide linkages, boronate esters, silyl ethers, imines, amidines, carbamates, acetals, ketals, phosphoramidates and the like. Further examples include di- and tri-phosphate-containing groups, for example a monophosphate-containing linker.

In some embodiments, the cleavable group may be within the linker structure, for example such as a disulfide linkage contained within the linker group. In some other embodiment, the cleavable group may be formed between a group present in the linker and part of the drug moiety or dendrimer structure, for example such as an amide formed between an acyl group present in the linker and an amine group present in the drug moiety.

In some embodiments, the drug moiety is covalently attached to a diacyl linker group of formula

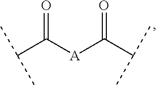

wherein A is a C₂-C₁₀ alkylene group which is optionally interrupted by at least one O, S, NH, or N(Me), or in which A is a heterocycle selected from the group consisting of tetrahydrofuran, tetrahydrothiophene, pyrrolidine and N-methylpyrrolidine.

As used herein, the term “alkyl” refers to straight (i.e., linear) or branched chain hydrocarbons ranging in size from one to 10 carbon atoms (i.e. C₁₋₁₀alkyl). Thus, alkyl moieties include, unless explicitly limited to smaller groups, moieties ranging in size, for example, from about one to about six carbon atoms or greater, such as, methyl, ethyl, n-propyl, iso-propyl and/or butyl, pentyl, hexyl, and higher isomers. In one example, the alkyl moiety is of one to 10 carbon atoms (i.e. C₁₋₁₀alkyl). In another example, the alkyl moiety is of 2 to 4 carbon atoms, for example 2, 3 or 4 carbon atoms.

As used herein, the term “alkylene” refers to straight (i.e. linear) or branched chain hydrocarbons ranging in size from 1 to 10 carbon atoms (i.e. C₁₋₁₀alkylene). Thus, alkylene moieties include, for example, —CH₂—, —CH₂CH₂—, —CH₂CH₂CH₂—, —CH₂CH(CH₃)—, —CH₂CH₂CH₂CH₂—, —CH₂CH(CH₃)CH₂—, and the like.

In some embodiments, the diacyl linker is:

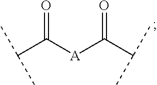

wherein A is a C₂-C₁₀ alkylene group (e.g straight chain or branched) which is interrupted by at least one O, S, NH, or N(Me).

In some embodiments, the diacyl linker is:

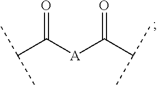

wherein A is a C₂-C₆ alkylene group (e.g. straight chain or branched) which is interrupted by at least one O, S, NH, or N(Me).

In some embodiments, the diacyl linker is selected from the group consisting of

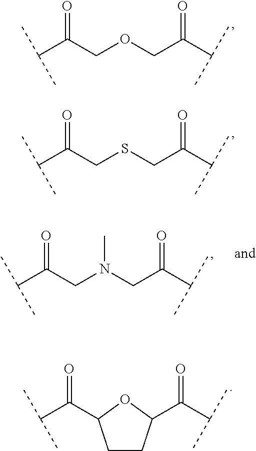

In some embodiments, the diacyl linker is:

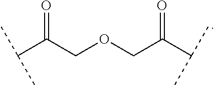

In some embodiments, the diacyl linker is:

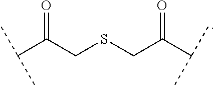

In some embodiments, the diacyl linker is:

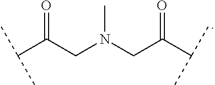

In some embodiments, the drug moiety is covalently attached to a diacyl linker group of formula

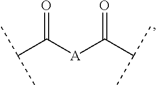

wherein A is a C₂-C₁₀ alkylene group.

In some embodiments, the diacyl linker is:

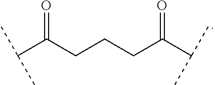

In some embodiments, the cleavable group is an amide group, for example formed between a C(O)— group present in the linker and a nitrogen atom present in the drug moiety.

In some embodiments, the cleavable group is an ester group, for example formed between a C(O)— group present in the linker and an oxygen atom present in the drug moiety.

In some embodiments, the cleavable group is a boronate ester, for example formed between a boron atom present in the linker and two oxygen atoms present in the drug moiety. For example, a linker group of the formula shown below may be used to link to a nitrogen atom of a surface building unit, and to two oxygen atoms of the drug moiety:

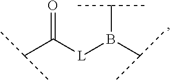

wherein L is an aryl (e.g. phenyl, for example para-substituted phenyl) group.

In some embodiments, the cleavable group is a silyl ether, for example formed between a silicon atom present in the linker and an oxygen atom present in the linker or present in the drug moiety. For example, a linker group of the formula shown below may be used to link to a nitrogen atom of a surface building unit, and to an oxygen atom of the drug moiety:

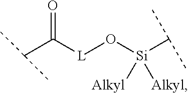

wherein L is a C₁₋₁₀ alkylene group optionally substituted by one or more of O, S, NH or NMe, and Alkyl is a C₁₋₆ alkyl.

In some embodiments, the cleavable group is a carbamate, for example formed between an O—C(O)— group present in the linker and a nitrogen atom present in the drug moiety. For example, a linker group of the formula shown below may be used to link to a nitrogen atom of a surface building unit, and to a nitrogen atom of the drug moiety:

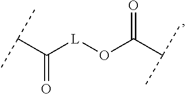

wherein L is a linker group, for example for example comprising one or more of a C₁₋₁₀alkylene group, a C₆₋₁₀aryl and a C₃₋₆ heterocyclic group, optionally interrupted by one or more of O, S, NH and NMe, and optionally substituted by ═O, e.g. it may be a group of the formula:

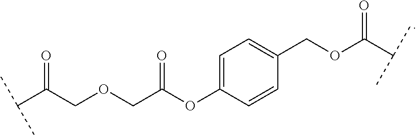

In some embodiments, the cleavable group is an imine or an amidine, for example formed by a double bond between a carbon atom present in the linker and a nitrogen atom present in the drug moiety. For example, a linker group of the formula shown below may be used to link to a nitrogen atom of a surface building unit, and to a nitrogen atom of the drug moiety:

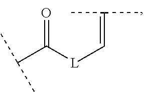

wherein L is a linker group, for example comprising one or more of a C₁₋₁₀alkylene group and a C₃₋₆ heterocyclic group, optionally interrupted by one or more of O, S, NH and NMe, and optionally substituted by ═O, e.g. it may be a group of the formula:

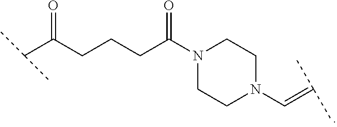

In some embodiments, the cleavable group is a phosphoramidate, for example formed between a nitrogen atom present in the linker and a phosphorous atom present in the drug moiety. For example, a linker group of the formula shown below may be used to link to a nitrogen atom of a surface building unit, and to a phosphorous atom of the drug moiety:

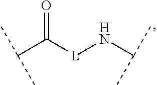

wherein L is a linker group, for example for example comprising one or more of a C₁₋₁₀alkylene group, a C₆₋₁₀aryl and a C₃₋₆ heterocyclic group, optionally interrupted by one or more of O, S, NH and NMe, and optionally substituted by ═O.

In some embodiments, the cleavable group is an acetal, for example formed between a carbon atom present in the linker and two oxygen atoms present in the drug moiety. For example, a linker group of the formula shown below may be used to link to a nitrogen atom of a surface building unit, and to two oxygen atoms of the drug moiety:

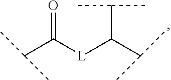

wherein L is a C₁₋₆ alkyl group.

In some embodiments, the dendrimer-drug conjugate comprises two or more different linkers (e.g. diacyl linkers). In some embodiment, the dendrimer-drug conjugate comprises two, three, or four different linkers. In one embodiment, the dendrimer-drug conjugate comprises two different linkers. In one embodiment, the dendrimer-drug conjugate comprises three different linkers. In one embodiment, the dendrimer-drug conjugate comprises four different linkers.

Having a dendrimer-drug conjugate with different linkers may provide for the release of the drug moiety at various rates. For example, the drug moiety may be released relatively quickly from the dendrimer-drug conjugate when a first linker is cleaved relatively quickly under physiological conditions. Similarly, for example, the drug moiety may be released relatively slowly from the dendrimer-drug conjugate when a second linker is cleaved relatively slowly under physiological conditions. This allows for fast release of a dose of drug moiety to the patient following administration, followed by a gradual, prolonged release of further drug moiety, such that, upon administration, therapeutically effective levels of drug moiety are provided quickly, and then maintained for a prolonged period of time.

In some embodiments, the dendrimer-drug conjugate comprises two different linkers (e.g. diacyl linkers), one of which releases drug moiety in vivo at a faster rate than the other.

In some embodiments, the dendrimer-drug conjugate comprises two or more linkers, wherein the two or more linkers are each a diacyl linker group of formula

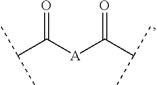

wherein A is a C₂-C₁₀ alkylene group which is optionally interrupted by at least one O, S, NH, or N(Me), or in which A is a heterocycle selected from the group consisting of tetrahydrofuran, tetrahydrothiophene, pyrrolidine and N-methylpyrrolidine.

In some embodiments, the dendrimer-drug conjugate comprises two or more diacyl linkers, wherein the two or more diacyl linkers are each a diacyl linker selected from the group consisting of:

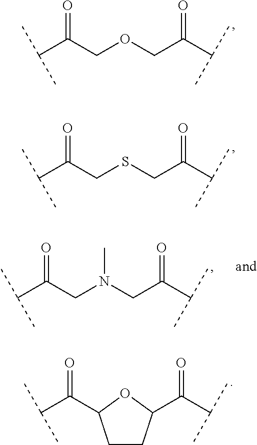

In some embodiments, the dendrimer-drug conjugate comprises two or more diacyl linkers, wherein the two or more diacyl linkers are each a diacyl linker selected from the group consisting of:

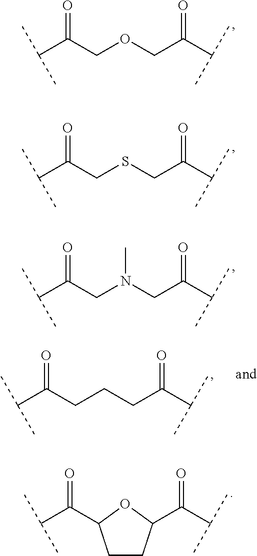

In some embodiments, the drug moiety is covalently attached to a diacyl linker via a linkage formed between an oxygen atom present as part of the drug moiety and a carbon atom of an acyl group present as part of the linker. The other acyl group of the diacyl linker forms an amide linkage with a nitrogen atom present in an outer building unit. In some embodiments, the drug moiety is covalently attached to a diacyl linker via a linkage formed between a nitrogen atom present as part of the drug moiety and a carbon atom of an acyl group present as part of the diacyl linker. The other acyl group of the diacyl linker forms an amide linkage with a nitrogen atom present in an outer building unit.

In some embodiments, the drug moiety has the substructure:

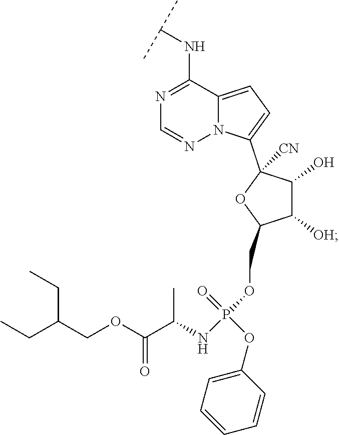

and is covalently attached to the diacyl linker via the nitrogen atom of the —NH₂ substituent of the adenosine-like ring, and wherein the diacyl linker is:

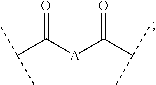

wherein A is a C₂-C₁₀ alkylene group which is optionally interrupted by at least one O, S, NH, or N(Me), or in which A is a heterocycle selected from the group consisting of tetrahydrofuran, tetrahydrothiophene, pyrrolidine and N-methylpyrrolidine.

In one embodiment, the first terminal group (T1) is:

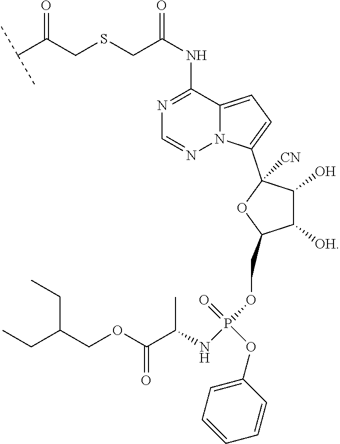

In one embodiment, the first terminal group (T1) is:

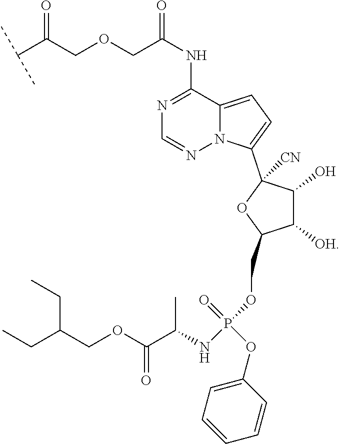

It has been found that, by the combination of particular cleavable linker groups with a drug moiety, that controlled release of the drug moiety comprising the Remdesivir nucleoside can be achieved, leading to high aqueous solubility, biological activity and effective pharmacokinetic properties.

### Second Terminal Group

The dendrimer-drug conjugates comprise a plurality of second terminal groups (T2) each comprising a hydrophilic polymeric group. The second terminal group may for example change the solubility profile of the dendrimer-drug conjugate, for example increasing the solubility of the dendrimer-drug conjugate in a pharmaceutically acceptable carrier. The second terminal group may for example provide for improved pharmacokinetic properties. The second terminal may for example provide for reduced immunogenicity and/or side effects.

The term hydrophilic polymeric group typically refers to a polymeric group which has a solubility in water at 25° C. of at least 25 mg/ml, more preferably at least 50 mg/ml, and still more preferably at least 100 mg/ml.

In some embodiments, the hydrophilic polymeric group comprises repeating units of amino acids, alkyloxy or alkyl(acyl)amino groups. In some embodiments, the hydrophilic polymeric group comprises repeating units of amino acids, such as sarcosine. In some embodiments, the hydrophilic polymeric group comprises repeating units of alkyloxy groups (e.g. the hydrophilic polymer is a PEG group). In some embodiments the hydrophilic polymer comprises repeating units of alkyl(acyl)amino groups (e.g. the hydrophilic polymer is a PEOX group).

In some embodiments, the hydrophilic polymeric group comprises at least 10 monomer units. In some embodiments, the hydrophilic polymeric group comprises up to 100 monomer units. In some embodiments, the hydrophilic polymeric group comprises from 10 to 100, or from 10 to 50 monomer units.

In some embodiments, the hydrophilic polymer comprises at least 50% of the MW of the dendrimer-drug conjugate. In some embodiments, the hydrophilic polymer comprises at least 60% of the MW of the dendrimer-drug conjugate.

In one embodiment, the second terminal group comprises a PEG group as the hydrophilic polymer. A PEG group is a polyethylene glycol group, i.e. a group comprising repeat units of the formula —CH₂CH₂O—. PEG materials used to produce the dendrimer of the present disclosure typically contain a mixture of PEGs having some variance in molecular weight (i.e., ±10%), and therefore, where a molecular weight is specified, it is typically an approximation of the average molecular weight of the PEG composition. For example, the term “PEG_{˜2100}” refers to polyethylene glycol having an average molecular weight of approximately 2100 Daltons, i.e. ±approximately 10% (PEG₁₈₉₀ to PEG₂₃₁₀). The term “PEG_{˜2300}” refers to polyethylene glycol having an average molecular weight of approximately 2300 Daltons, i.e. ±approximately 10% (PEG₂₀₇₀ to PEG₂₅₃₀). Three methods are commonly used to calculate MW averages: number average, weight average, and z-average molecular weights. As used herein, the phrase “molecular weight” is intended to refer to the weight-average molecular weight which can be measured using techniques well-known in the art including, but not limited to, NMR, mass spectrometry, matrix-assisted laser desorption ionization time of flight (MALDI-TOF), gel permeation chromatography or other liquid chromatography techniques, light scattering techniques, ultracentrifugation and viscometry.

In some embodiments, the second terminal groups comprise PEG groups having an average molecular weight of between about 200 and 5000 Daltons. In some embodiments, the second terminal groups comprise PEG groups having an average molecular weight of at least 750 Daltons. In some embodiments, the second terminal groups comprise PEG groups having an average molecular weight in the range of from 500 to 2500 Daltons. In some embodiments, the second terminal groups comprise PEG groups having an average molecular weight in the range of from 1000 to 2500 Daltons. In some embodiments, the second terminal groups comprise PEG groups having an average molecular weight in the range of from 1500 to 2500 Daltons. In some embodiments, the second terminal groups comprise PEG groups having an average molecular weight in the range of from 1900 to 2300 Daltons. In some embodiments, the second terminal groups comprise PEG groups having an average molecular weight in the range of from 2100 to 2500 Daltons. In some embodiments, the second terminal groups comprise PEG groups having an average molecular weight of about 1900, about 2000, about 2100, about 2200, about 2300, about 2400 or about 2500 Daltons.

In some embodiments, the second terminal groups comprise PEG groups having an average molecular weight in the range of from 500 to 1500 Daltons. In some embodiments, the second terminal groups comprise PEG groups having an average molecular weight in the range of from 800 to 1300 Daltons. In some embodiments, the second terminal groups comprise PEG groups having an average molecular weight in the range of from 750 to 1200 Daltons. In some embodiments, the second terminal groups comprise PEG groups having an average molecular weight of about 800, about 900, about 1000, about 1100, about 1200 or about 1300 Daltons.

In some embodiments, the PEG group has a polydispersity index (PDI) of between about 1.00 and about 1.50, between about 1.00 and about 1.25, or between about 1.00 and about 1.10. In some embodiments, the PEG group has a polydispersity index (PDI) of about 1.05. The term “polydispersity index” refers to a measure of the distribution of molecular mass in a given polymer sample. The polydispersity index (PDI) is equal to the weight average molecular weight (M_{w}) divided by the number average molecular weight (Mₙ) and indicates the distribution of individual molecular masses in a batch of polymers. The polydispersity index (PDI) has a value equal to or greater than one, but as the polymer approaches uniform change length and average molecular weight, the polydispersity index (PDI) will be closer to one.

Where the second terminal groups comprise a PEG group, the PEG groups may be linear or branched. If desired, an end-capped PEG group may be used. In some embodiments, the PEG group is a methoxy-terminated PEG.

In one embodiment, the second terminal group comprises a PEOX group. A PEOX group is a polyethyloxazoline group, i.e. a group comprising repeat units of the formula

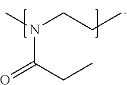

PEOX groups are so named since they can be produced by polymerisation of ethyloxazoline. PEOX materials used to produce the dendrimer of the present disclosure typically contain a mixture of PEOXs having some variance in molecular weight (i.e., ±10%), and therefore, where a molecular weight is specified, it is typically an approximation of the average molecular weight of the PEOX composition. In some embodiments, the second terminal groups comprise PEOX groups having an average molecular weight of at least 750 Daltons, at least 1000 Daltons, or at least 1500 Daltons. In some embodiments, the second terminal groups comprise PEOX groups having an average molecular weight in the range of from 750 Daltons to 2500 Daltons, or from 1000 Daltons to 2000 Daltons. If desired, an end-capped PEOX group may be used. In some embodiments, the PEOX group is a methoxy-terminated PEOX.

In some embodiments, the second terminal group comprises a polysarcosine group, i.e. a group comprising repeat units of the formula

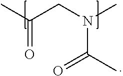

In some embodiments, the second terminal groups comprise polysarcosine groups having an average molecular weight of at least 750 Daltons, at least 1000 Daltons, or at least 1500 Daltons. In some embodiments, the second terminal groups comprise polysarcosine groups having an average molecular weight in the range of from 750 Daltons to 2500 Daltons, or from 1000 Daltons to 2500 Daltons.

The second terminal group may be attached to the outer building unit via any suitable means. In some embodiments, a linking group is used to attach the hydrophilic polymeric group to the outer building unit.

Where required, the second terminal groups are typically attached via use of a second terminal group precursor which contains a reactive group that is reactive with an amine group, such as a reactive acyl group (which can form an amide bond), or an aldehyde (which can form an amine group under reductive amination conditions).

In some embodiments, the second terminal groups each comprise a PEG group covalently attached to a PEG linking group (L1) via an ether linkage formed between a carbon atom present in the PEG group and an oxygen atom present in the PEG linking group, and each second terminal group is covalently attached to a building unit via an amide linkage formed between a nitrogen atom present in a building unit and the carbon atom of an acyl group present in the PEG linking group.

In some embodiments, the second terminal groups are each

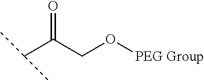

and wherein the PEG group is a methoxy-terminated PEG having an average molecular weight in the range of from about 500 to about 2500 Daltons.

In some embodiments, the second terminal groups are each

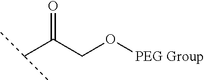

and wherein the PEG group is a methoxy-terminated PEG having an average molecular weight in the range of from about 1750 to about 2500 Daltons.

In some embodiments, the second terminal groups each comprise a PEOX group covalently attached to a PEOX linking group (L1′) via a linkage formed between a nitrogen atom present in the PEOX group and a carbon atom present in the PEOX linking group, and each second terminal group is covalently attached to a building unit via an amide linkage formed between a nitrogen atom present in a building unit and the carbon atom of an acyl group present in the PEOX linking group. In some embodiments, the
second terminal groups are each

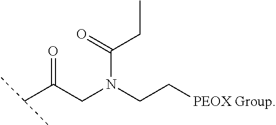

In some embodiments, the second terminal groups are each polysarcosine groups, e.g. of the formula:

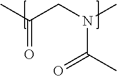

and are attached to a building unit via an amide linkage formed between a nitrogen atom present in a building unit and the carbon atom of an acyl group present in the polysarcosine group.

In some embodiments of the dendrimer-drug conjugates of the present disclosure, at least one half of the outer building units have one nitrogen atom covalently attached to a first terminal group and have one nitrogen atom covalently attached to a second terminal group. The dendrimers can thus be considered to have controlled stoichiometry and/or topology. For example, the dendrimer-drug conjugates are typically produced using synthetic processes that allow for a high degree of control over the number and arrangement of first and second terminal groups present on the dendrimer-drug conjugates. The dendrimer-drug conjugates may be synthesised using orthogonal protecting groups to allow for conjugation of the terminal groups to the outer building unit in a predefined or controlled manner. In some embodiments, at least two thirds of the outer building units have one nitrogen atom covalently attached to a first terminal group and have one nitrogen atom covalently attached to a second terminal group. In some embodiments, at least 75%, at least 80%, at least 85%, or at least 90%, of the outer building units have one nitrogen atom covalently attached to a first terminal group and have one nitrogen atom covalently attached to a second terminal group. In some embodiments, each functionalised outer building unit contains one first terminal group and one second terminal group.

In some embodiments, the dendrimer comprises surface units comprising an outer building unit attached to a first terminal group and a second terminal group, the surface units having a structure selected from the group consisting of:

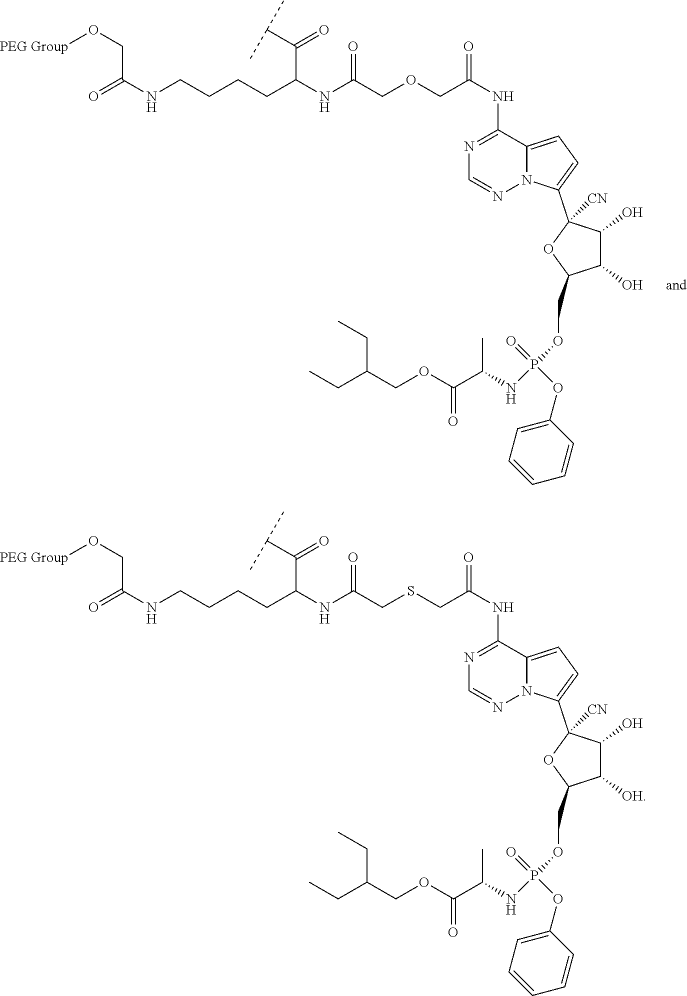

Within those embodiments, in some examples the PEG group is a methoxy-terminated PEG having an average molecular weight in the range of from about 500 to 2500 Daltons, or about 1500 to 2500 Daltons, or about 750 to 1200 Daltons.

The building units are lysine residues or analogues. Lysine has an alpha-nitrogen atom (a nitrogen which is attached to a carbon atom which is α- to the carbon atom which is part of the carbonyl group present in lysine) and an epsilon-nitrogen atom (a nitrogen which is attached to a carbon atom which is ε- to the carbon atom which is part of the carbonyl group present in lysine).

In many cases, a population of dendrimer-drug conjugates that has been functionalised at the dendrimer surface contain a random stoichiometry and topology of functional groups. For example, the reaction of a population of dendrimer-drug conjugate molecules containing, e.g., 64 reactive surface groups with one or more reactive functional groups may result in a diverse population of functionalised dendrimer-drug conjugate products, with some dendrimer-drug conjugate products containing higher numbers of functional groups than others. In cases where there are multiple different surface groups available for reaction with a reactive functional group, a wide distribution of dendrimer-drug conjugate products having different surface topologies may also be obtained.

In the present case, in some embodiments the dendrimer-drug conjugate has controlled stoichiometry and/or controlled topology with regard to the first terminal groups and second terminal groups. For example, in some embodiments alpha-nitrogen atoms of outer building units are attached to first terminal groups and epsilon-nitrogen atoms of outer building units are attached to second terminal groups. In other embodiments, epsilon-nitrogen atoms of outer building units are attached to first terminal groups and alpha-nitrogen atoms of outer building units are second terminal groups.

The present dendrimer-drug conjugate scaffolds, intermediates, and processes, allow for high loadings of drug moiety comprising the Remdesivir nucleoside to be incorporated into the dendrimer-drug conjugate. Such dendrimer-drug conjugates are also considered to facilitate therapeutically effective levels of the drug moiety to be released over an extended period of time following administration, and thus may decrease the frequency and/or number of administrations required.

Drug loading (% w/w) can be calculated by multiplying the molecular weight of the drug moiety by the number of drug moiety groups loaded on to the dendrimer, divided by the total molecular weight of the drug-dendrimer conjugate construct. In some embodiments, the drug moiety comprises 15 to 40% of the MW of the dendrimer-drug conjugate. In some embodiments, the drug moiety comprises 15 to 25% of the MW of the dendrimer-drug conjugate. In some embodiments, the drug moiety comprises at least 15% of the MW of the dendrimer-drug conjugate.

In some embodiments, for example wherein the dendrimer has 5 generations of building units, the dendrimer has from 24 to 32, from 26 to 32, from 28 to 32, from 30 to 32, from 24 to 30, from 26 to 30, from 28 to 30, from 26 to 30, from 26 to 28, or from 28 to 30 surface units, the surface units comprising an outer building unit attached to a first terminal group and attached to a second terminal group.

In some embodiments, from 26 to 32, or from 27 to 32, or from 28 to 32 first terminal groups are covalently attached to nitrogen atoms present on outer building units. In some embodiments, from 26 to 32, or from 27 to 32, or from 28 to 32 first terminal groups are covalently attached to alpha-nitrogen atoms present on outer building units

In some embodiments, from 26 to 32, or from 27 to 32, or from 28 to 32 second terminal groups are covalently attached to nitrogen atoms present on outer building units. In some embodiments, from 26 to 32, or from 27 to 32, or from 28 to 32 second terminal groups are covalently attached to epsilon-nitrogen atoms present on outer building units.

In some embodiments, the conjugate has three generations of building units which are complete generations, and wherein the outer generation of building units provides 16 nitrogen atoms for covalent attachment to a first terminal group or a second terminal, wherein from 6 to 8 first terminal groups are covalently attached to one of said nitrogen atoms, and wherein from 6 to 8 second terminal groups are each covalently attached to one of said nitrogen atoms.

In some embodiments, the conjugate has four generations of building units which are complete generations, and wherein the outer generation of building units provides 32 nitrogen atoms for covalent attachment to a first terminal group or a second terminal, wherein from 12 to 16 first terminal groups are covalently attached to one of said nitrogen atoms, and wherein from 12 to 16 second terminal groups are each covalently attached to one of said nitrogen atoms.

In some embodiments, the conjugate has five generations of building units which are complete generations, and wherein the outer generation of building units provides 64 nitrogen atoms for covalent attachment to a first terminal group or a second terminal, wherein from 26 to 32 first terminal groups are covalently attached to one of said nitrogen atoms, and wherein from 26 to 32 second terminal groups are each covalently attached to one of said nitrogen atoms.

In some embodiments, no more than one quarter of the nitrogen atoms present in the outer generation of building units are unsubstituted. In some embodiments, no more than one fifth of the nitrogen atoms present in said outer generation of building units are unsubstituted. In some embodiments, no more than one sixth of the nitrogen atoms present in said outer generation of building units are unsubstituted. In some embodiments, no more than one eighth of the nitrogen atoms present in said outer generation of building units are unsubstituted. In some embodiments, no more than one tenth of the nitrogen atoms present in said outer generation of building units are unsubstituted.

In some embodiments, no more than 20 nitrogen atoms present in the outer generation of building units are unsubstituted. In some embodiments, no more than 10 nitrogen atoms present in the outer generation of building units are unsubstituted. In some embodiments, no more than 5 nitrogen atoms present in the outer generation of building units are unsubstituted. In some embodiments, no more than 3 nitrogen atoms present in the outer generation of building units are unsubstituted. In some embodiments, no more than 2 nitrogen atoms present in the outer generation of building units are unsubstituted. In some embodiments, no more than 1 nitrogen atom present in the outer generation of building units are unsubstituted. In some embodiments, substantially all of the nitrogen atoms present in the outer generation of building units are substituted.

It will be appreciated that, in addition to the drug moiety and the PEG or PEOX group, further terminal groups can be attached to the dendrimer. Thus, in some embodiments, the dendrimer-drug conjugate comprises one or more third terminal groups. In some embodiments, the third terminal group comprises a residue of a further therapeutic agent, such as a therapeutic agent which does not comprise a Remdesivir nucleoside. For example, the third terminal group may comprise a residue of a further therapeutic agent used for therapy of a viral condition. The residue of a further therapeutic agent may be attached via a linker (e.g., a cleavable linker), for example. At least one half of the outer building units have one nitrogen atom covalently attached to a first terminal group and have one nitrogen atom covalently attached to a second terminal group. In some embodiments, where the dendrimer-drug conjugate comprises one or more third terminal groups, the third terminal groups may be attached to the nitrogen atom of an outer building unit which is not covalently attached to a first or second terminal group.

In some embodiments, alpha-nitrogen atoms of outer building units are attached to third terminal groups. In some embodiments, epsilon-nitrogen atoms of outer building units are attached to third terminal groups.

In some embodiments, the dendrimer-drug conjugate is:

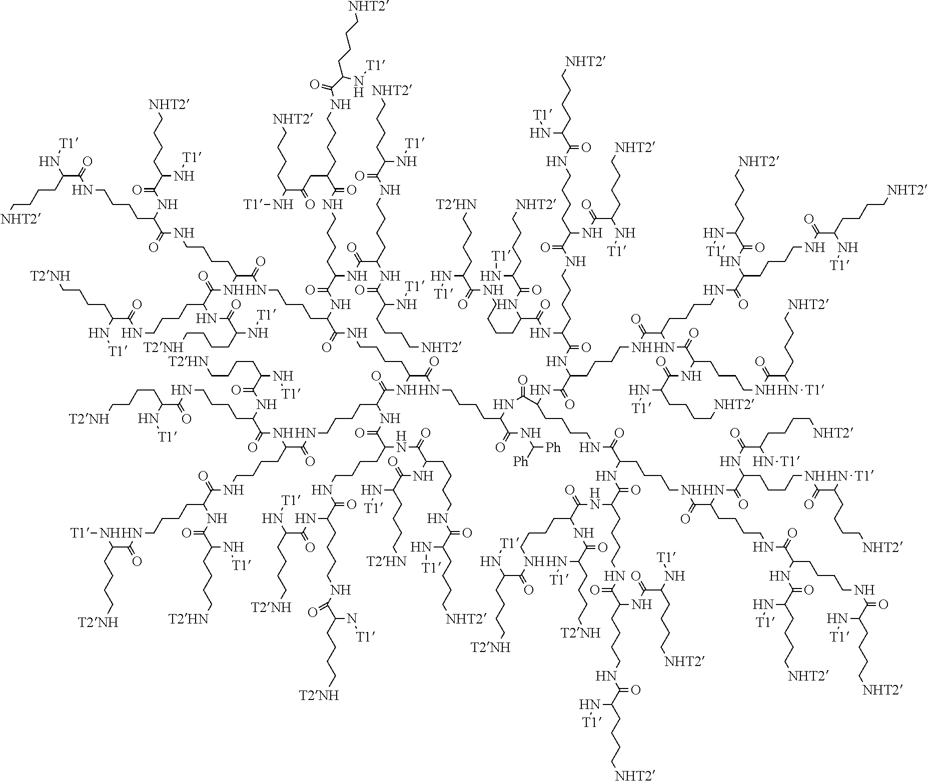

in which T1′ represents a group selected from the group consisting of hydrogen, and

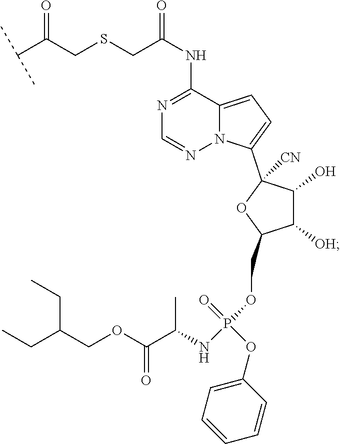

and wherein less than 10 of T1′ are hydrogen; and
T2′ represents a second group which is

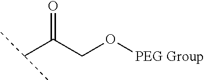

wherein the PEG group is a methoxy-terminated PEG having an average molecular weight in the range of from 500 to 2500 Daltons, or T2′ represents H, and wherein less than 10 of T2′ are H.

In some embodiments, the dendrimer-drug conjugate is:

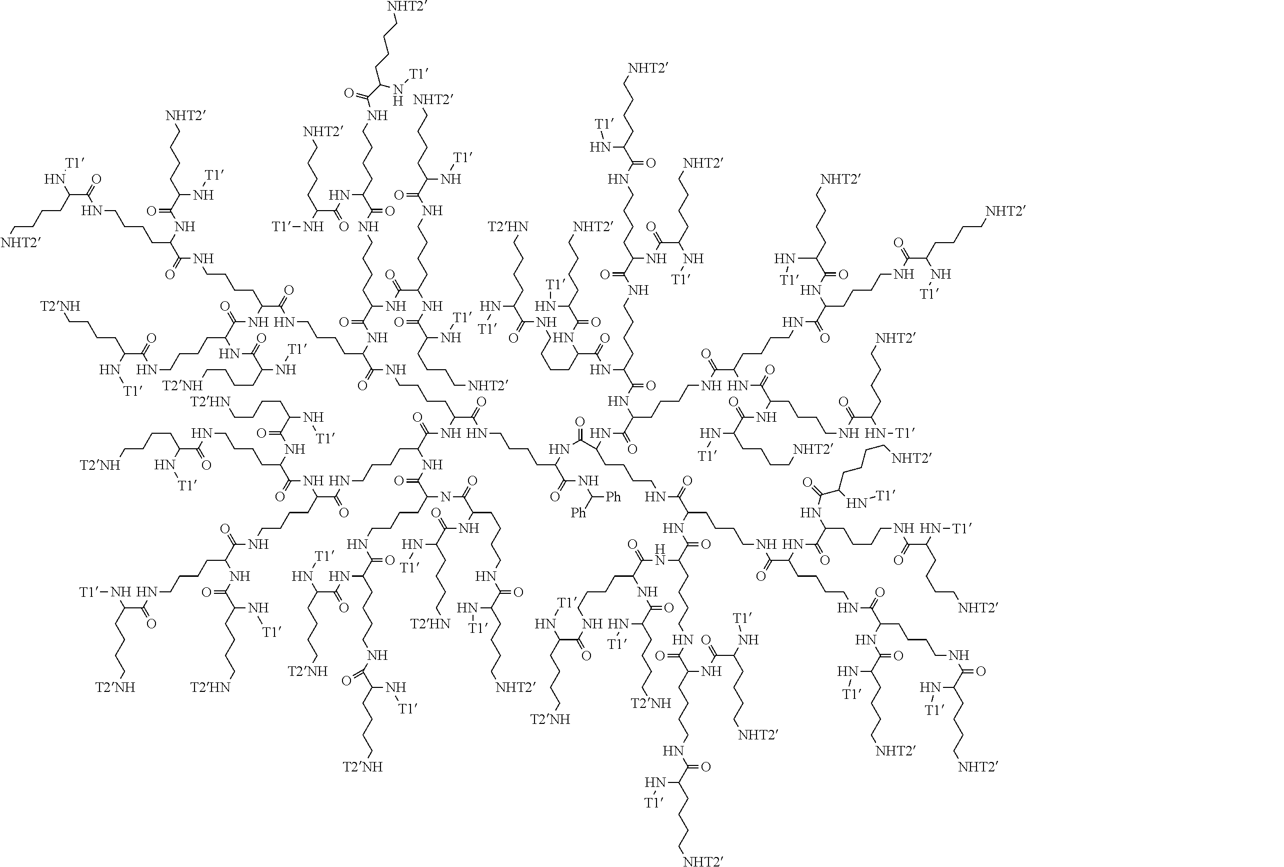

in which T1′ represents a group selected from the group consisting of hydrogen, and

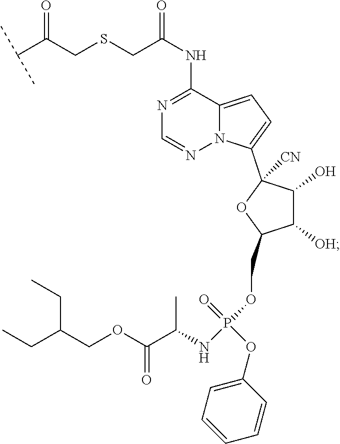

and wherein less than 5 of T1′ are hydrogen; and
T2′ represents a second group which is

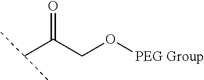

wherein the PEG group is a methoxy-terminated PEG having an average molecular weight in the range of from 500 to 2500 Daltons, or T2′ represents H, and wherein less than 5 of T2′ are H.

In some embodiments, the dendrimer-drug conjugate has a molecular weight in the range of from 25 to 300 kDa, or from 40 to 300 kDa, or from 75 to 200 kDa, or from 90 to 150 kDa. In some embodiments, the dendrimer-drug conjugate has a molecular weight in the range of from 35 to 100 kDa. In one example, the dendrimer-drug conjugate has a molecular weight in the range of from 35 to 45 kDa, or in the range of from 50 to 60 kDa, or in the range of from 85 to 95 kDa.

### Therapeutic Methods

There is also provided a dendrimer-drug conjugate or pharmaceutical composition as described herein for use in therapy.

The conjugates and pharmaceutical compositions described herein find use in therapy of viral infections, such as coronavirus infections.

Accordingly, there is provided a dendrimer-drug conjugate or pharmaceutical composition as described herein for use in the prevention and/or treatment of a viral infection.

There is also provided a method of preventing and/or treating a viral infection comprising administering to a subject in need thereof a therapeutically effective amount of a dendrimer-drug conjugate or a composition, as defined herein.

There is also provided use of a dendrimer-drug conjugate or a composition, as defined herein, in the manufacture of a medicament for the treatment and/or prevention of a viral infection.

### Coronavirus (CoV)

As used herein, “Coronaviridae”, known by the common name of “Coronavirus” or “CoV” are enveloped, positive sense, single-stranded RNA viruses. In some embodiment, the viral infection is a coronavirus (CoV) infection. In some embodiments, the virus is an RNA infection. The family of Coronaviridae viruses belong to the broader realm of Riboviria viruses. In some embodiments, the virus is a Riboviria infection. The family of Coronaviridae viruses belong to the broader kingdom of Orthornavirae viruses.

In some embodiments, the virus is an Orthornavirae infection. There are two subfamilies of Coronaviridae; Letovirinae and Orthocoronavirinae. The phylogeny of coronaviruses is outlined in Coronaviridae Study Group (2020).

In one embodiment, the CoV is selected from the genera Alphacoronavirus (alphaCoV), Betacoronavirus (betaCoV), Gammacoronavirus (gammaCoV) and Deltacoronavirus (deltaCoV).

In one embodiment, the alphaCoV is selected from coronavirus 229E (HCoV-229E), human coronavirus NL63 (HCoV-NL63), transmissible gastroenteritis virus (TGEV), porcine epidemic diarrhea virus (PEDV), feline infectious peritonitis virus (FIPV) and canine coronavirus (CCoV).

In one embodiment, the betaCoV is selected from human coronavirus HKU1 (HCoV-HKU1), Human coronavirus OC43 (HCoV-OC43), Severe acute respiratory syndrome-related coronavirus (SARS-CoV), Severe acute respiratory syndrome-related coronavirus-2 (SARS-Cov-2), Middle-East respiratory syndrome-related coronavirus (MERS-CoV), murine hepatitis virus (MHV) and/or bovine coronavirus (BCoV).

In one embodiment, the CoV is capable of infecting a human.

In one embodiment, the CoV capable of infecting a human is selected from the group consisting of SARS-CoV-2, HCoV-OC43, HCoV-HKU1, HCoV-229E, HCoV-NL63, SARS-CoV, and MERS-CoV, and a subtype or strain or variant thereof.

In one embodiment, the CoV has a death rate in humans of about 0.001 to about 10%. In one embodiment, the CoV has a death rate in humans of about 0.01 to about 9%. In one embodiment, the CoV has a death rate in humans of about 0.01 to about 8%. In one embodiment, the CoV has a death rate in humans of about 0.01 to about 7%. In one embodiment, the CoV has a death rate in humans of about 0.01 to about 6%.

In one embodiment, the CoV has a median daily time-varying basic reproduction number (Rt) in humans of about 1.3 to about 5 when minimal social restrictions are in place. In one embodiment, the CoV has an Rt in humans of about 1.4 to about 4 when minimal social restrictions are in place. In one embodiment, the CoV has an Rt in humans of about 1.4 to about 3 when minimal social restrictions are in place. In one embodiment, the CoV has an Rt in humans of about 1.4 to about 2.6 when minimal social restrictions are in place. In an embodiment, the Rt is calculated as described in Kucharski et al 2020.

In one embodiment, the CoV is SARS-CoV-2 or a subtype or variant thereof. In one embodiment, the SARS-CoV-2 is SARS-CoV-2 subtype L as described in Tang et al., 2020. In one embodiment, the SARS-CoV-2 is SARS-CoV-2 subtype S as described in Tang et al., 2020. In an embodiment, SARS-CoV-2 is SARS-CoV-2 hCoV-19/Australia/VIC01/2020. In one embodiment, SARS-COV-2 comprises the sequences as described in NCBI Reference Sequence: NC_045512.2. In one embodiment, SARS-CoV-2 comprises the sequence as described in GenBank: MN908947.3 or a variant thereof. Examples of SARS-CoV-2 variants are described, for example, in Shen et al., 2020 and Tang et al., 2020. Foster et al (2020) have found 3 variants, A, B and C, based on genomic analysis. In some embodiments, the SARS-CoV-2 is SARS-CoV-2 variant A. In some embodiments, the SARS-CoV-2 is SARS-CoV-2 variant B. In some embodiments, the SARS-CoV-2 is SARS-CoV-2 variant C.

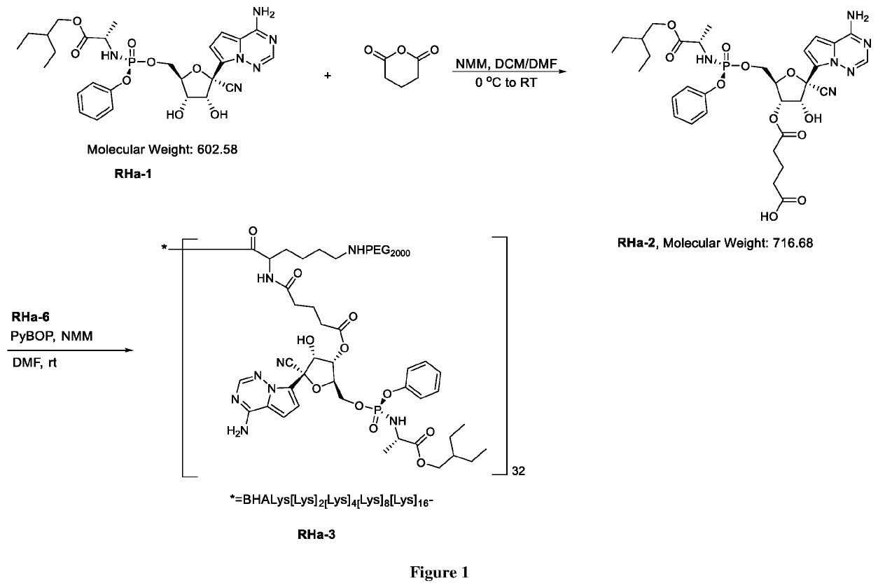

In one embodiment, the variant is at least 90% identical to the parental sequence. In one embodiment, the variant is at least 92% identical to the parental sequence. In one embodiment, the variant is at least 93% identical to the parental sequence. In one embodiment, the variant is at least 94% identical to the parental sequence. In one embodiment, the variant is at least 95% identical to the parental sequence. In one embodiment, the variant is at least 96% identical to the parental sequence. In one embodiment, the variant is at least 97% identical to the parental sequence. In one embodiment, the variant is at least 98% identical to the parental sequence. In one embodiment, the variant is at least 99% identical to the parental sequence. In some embodiments, the variant is at least 99.5% identical to the parental sequence. In some embodiments, the variant is at least 99.8% identical to the parental sequence. In some embodiments, the parental strain is SARS-CoV-2 hCoV-19/Australia/VIC01/2020. In some embodiment, the parental strain is BetaCoV/Wuhan/WIV04/2019. In some embodiments, the parental strain of SARS-COV-2 is a strain listed in supplementary FIG. 1 and supplementary Table 1 of Wang et al 2020. In some embodiments, the strain comprises a variation as described in Ugurel et al 2020.

CoV infections cause can cause respiratory, enteric, hepatic, and neurological diseases in different animal species, including camels, cattle, cats, and bats.

CoV can be transmitted from one individual to another through contact of viral droplets with mucosa. Typically, viral droplets are airborne and inhaled via the respiratory tract including the nasal airway. In some examples, during an infection, CoV can be found in the upper respiratory tract, for example the nasal passages and/or eyes. In one embodiment, during an infection, CoV is found in the nasal passages. In one embodiment, during an infection, CoV is found in the eyes. In one embodiment, during an infection, CoV is found in the aqueous humor surrounding the eye. In some examples, during an infection, CoV can be found in the lower respiratory tract, for example the bronchi and/or alveoli. In one example, during an infection, CoV is found in the bronchi. In one example, during an infection, CoV is found in the alveoli.

In one embodiment, a CoV infection can cause one or more symptoms selected from the group consisting of fever, cough (e.g., dry cough), fatigue, sore throat, shortness of breath, viral shedding respiratory insufficiency, runny nose, nasal congestion, conjunctivitis, loss of taste (hypogeusia) and/or smell (anosmia), rash, discolouration of extremities (i.e., fingers, toes) malaise, bronchitis, headache, muscle ache and/or pain, dyspnea, moderate pneumonia, severe pneumonia, chest pain/pressure, loss of speech and/or movement, and acute respiratory distress syndrome (ARDS). In some embodiments, the ARDS is selected from mild ARDS (defined as 200 mmHg<PaO2/FiO2≤300 mmHg), moderate ARDS (defined as 100 mmHg<PaO2/FiO2≤200 mmHg) and severe ARDS (defined as PaO2/FiO2≤100 mmHg).

In one embodiment, a SARS-CoV-2 infection can cause one or more symptoms selected from the group consisting of fever, cough, sore throat, shortness of breath, viral shedding, respiratory insufficiency, runny nose, nasal congestion, malaise, bronchitis, headache, muscle pain, dyspnea, moderate pneumonia, severe pneumonia, and acute respiratory distress syndrome (ARDS).

In one embodiment, administration of the dendrimer-drug conjugate reduces the NEWS (National Early Warning Score) or NEWS2 score of the individual. In one embodiment, the dendrimer-drug conjugate reduces the viral load of the individual. A person skilled in the art will appreciate that viral load can be measured by any method known to a person skilled in the art including, for example, measurement by Quantitative reverse transcription PCR (RT-qPCR) to the relevant viral nucleotide sequences. In one embodiment, viral load is reduced to above 20CT (cycle threshold), or reduced to above 30CT, or reduced to above 35CT, or reduced to above 40CT.

In one embodiment, the dendrimer-drug conjugate reduces the coronavirus antibody titre of the individual. In one embodiment, the IgA, IgG and/or IgM antibody titre is measured by ELISA, and is reduced to below detectable levels. In some embodiments, the antibody is to protein S or N. In some embodiments, the sample tested is taken from oral swabs, nasal swabs, blood sample, throat swabs or lung fluid.

In some embodiments, the viral infection is a Coronoavirus infection, an influenza infection (e.g. influenza A viral infection), an RNA viral infection or an ebolavirus infection.

In one embodiment, the CoV is not SARS-CoV.

As discussed above, in some embodiments, the viral infection is a Coronavirus (CoV) infection.

As discussed supra, the Coronavirus (CoV) infection may be selected from the group consisting of severe acute respiratory syndrome-related coronavirus-2 (SARS-CoV-2), human coronavirus OC43 (HCoV-OC43), human coronavirus HKU1 (HCoV-HKU1), human coronavirus 229E (HCoV-229E), human coronavirus NL63 (HCoV-NL63), severe acute respiratory-related coronavirus (SARS-CoV), and middle-east respiratory syndrome-related coronavirus (MERS-CoV), and subtypes or variants thereof.

Accordingly, in some embodiments, the Coronavirus (CoV) infection is selected from the group consisting of severe acute respiratory syndrome-related coronavirus-2 (SARS-CoV-2), human coronavirus OC43 (HCoV-OC43), human coronavirus HKU1 (HCoV-HKU1), human coronavirus 229E (HCoV-229E), human coronavirus NL63 (HCoV-NL63), severe acute respiratory-related coronavirus (SARS-CoV), and middle-east respiratory syndrome-related coronavirus (MERS-CoV), and subtypes or strains or variants thereof. In one embodiment, the Coronavirus (CoV) infection is SARS-CoV-2.

In some embodiments, there is also provided a method of preventing or reducing the likelihood or severity of a symptom associated with a Coronavirus (CoV) infection, comprising administering to a subject in need thereof a therapeutically effective amount of a dendrimer-drug conjugate or a composition, as defined herein. As discussed supra, a symptom associated with a Coronavirus (CoV) infection, includes, but is not limited to, any one or more selected from the group consisting of fever, cough, sore throat, shortness of breath, viral shedding, respiratory insufficiency, runny nose, nasal congestion, bronchitis, headache, muscle pain, dyspnea, moderate pneumonia, severe pneumonia, and acute respiratory distress syndrome (ARDS).

In one embodiment, there is provided a method of preventing or reducing fever associated with a Coronavirus (CoV) infection, comprising administering to a subject in need thereof a therapeutically effective amount of a dendrimer-drug conjugate or a composition, as defined herein. In one embodiment, there is provided a method of preventing or reducing cough associated with a Coronavirus (CoV) infection, comprising administering to a subject in need thereof a therapeutically effective amount of a dendrimer-drug conjugate or a composition, as defined herein. In one embodiment, there is provided a method of preventing or reducing a sore throat associated with a Coronavirus (CoV) infection, comprising administering to a subject in need thereof a therapeutically effective amount of a dendrimer-drug conjugate or a composition, as defined herein. In one embodiment, there is provided a method of preventing or reducing shortness of breath associated with a Coronavirus (CoV) infection, comprising administering to a subject in need thereof a therapeutically effective amount of a dendrimer-drug conjugate or a composition, as defined herein. In one embodiment, there is provided a method of preventing or reducing respiratory insufficiency associated with a Coronavirus (CoV) infection, comprising administering to a subject in need thereof a therapeutically effective amount of a dendrimer-drug conjugate or a composition, as defined herein. In one embodiment, there is provided a method of preventing or reducing a runny nose associated with a Coronavirus (CoV) infection, comprising administering to a subject in need thereof a therapeutically effective amount of a dendrimer-drug conjugate or a composition, as defined herein. In one embodiment, there is provided a method of preventing or reducing nasal congestion associated with a Coronavirus (CoV) infection, comprising administering to a subject in need thereof a therapeutically effective amount of a dendrimer-drug conjugate or a composition, as defined herein. In one embodiment, there is provided a method of preventing or reducing bronchitis associated with a Coronavirus (CoV) infection, comprising administering to a subject in need thereof a therapeutically effective amount of a dendrimer-drug conjugate or a composition, as defined herein. In one embodiment, there is provided a method of preventing or reducing headache associated with a Coronavirus (CoV) infection, comprising administering to a subject in need thereof a therapeutically effective amount of a dendrimer-drug conjugate or a composition, as defined herein. In one embodiment, there is provided a method of preventing or reducing muscle pain associated with a Coronavirus (CoV) infection, comprising administering to a subject in need thereof a therapeutically effective amount of a dendrimer-drug conjugate or a composition, as defined herein. In one embodiment, there is provided a method of preventing or reducing dyspnea associated with a Coronavirus (CoV) infection, comprising administering to a subject in need thereof a therapeutically effective amount of a dendrimer-drug conjugate or a composition, as defined herein. In one embodiment, there is provided a method of preventing or reducing moderate pneumonia associated with a Coronavirus (CoV) infection, comprising administering to a subject in need thereof a therapeutically effective amount of a dendrimer-drug conjugate or a composition, as defined herein. In one embodiment, there is provided a method of preventing or reducing severe pneumonia associated with a Coronavirus (CoV) infection, comprising administering to a subject in need thereof a therapeutically effective amount of a dendrimer-drug conjugate or a composition, as defined herein. In one embodiment, there is provided a method of preventing or reducing acute respiratory distress syndrome (ARDS) associated with a Coronavirus (CoV) infection, comprising administering to a subject in need thereof a therapeutically effective amount of a dendrimer-drug conjugate or a composition, as defined herein. In one embodiment, there is provided a method of preventing or reducing neurological symptoms associated with a Coronavirus (CoV) infection, comprising administering to a subject in need thereof a therapeutically effective amount of a dendrimer-drug conjugate or a composition, as defined herein. In one embodiment the neurological symptoms are cognitive dysfunction including short term memory loss or confusion/delirium. Other symptoms include impaired vision and hearing, loss of taste and smell, insomnia, or fatigue. As used herein, the term “confusion” and/or “delirium” refers to a decline in mental status. Such confusion/delirium may present, for example, as a subject being disoriented, distracted, and having difficulty in concentrating.

In one embodiment, there is provided a method of preventing or reducing viral shedding associated with a Coronavirus (CoV) infection, comprising administering to a subject in need thereof a therapeutically effective amount of a dendrimer-drug conjugate or a composition, as defined herein. In one embodiment, the method, as described herein, may reduce viral shedding by at least 10, 20, 30, 40, 50, or 75%, compared to viral shedding in an untreated subject with a Coronavirus (CoV) infection. In one embodiment, the method, as described herein, may reduce viral shedding by between about 10 and 90%, between about 20 and 75%, between about 30 and 70%, or between about 40 and 70%, compared to viral shedding in an untreated subject with a Coronavirus (CoV) infection.

In some embodiments, the dendrimer-drug conjugate that is used in therapy (e.g. in therapy of a viral infection) has a core which is:

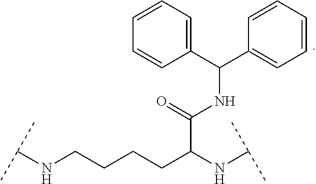

In some embodiments, the dendrimer-drug conjugate which is used in therapy (e.g. in therapy of a viral infection) has building units which are each:

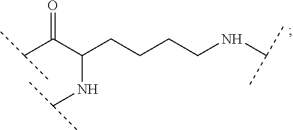

wherein the acyl group of each building unit provides a covalent attachment point for attachment to the core or to a previous generation building unit; and wherein each nitrogen atom provides a covalent attachment point for covalent attachment to a subsequent generation building unit, a first terminal group or a second terminal group.

In some embodiments, the dendrimer-drug conjugate which is used in therapy (e.g. in therapy of a viral infection) has first terminal groups (T1) which are selected from the group consisting of

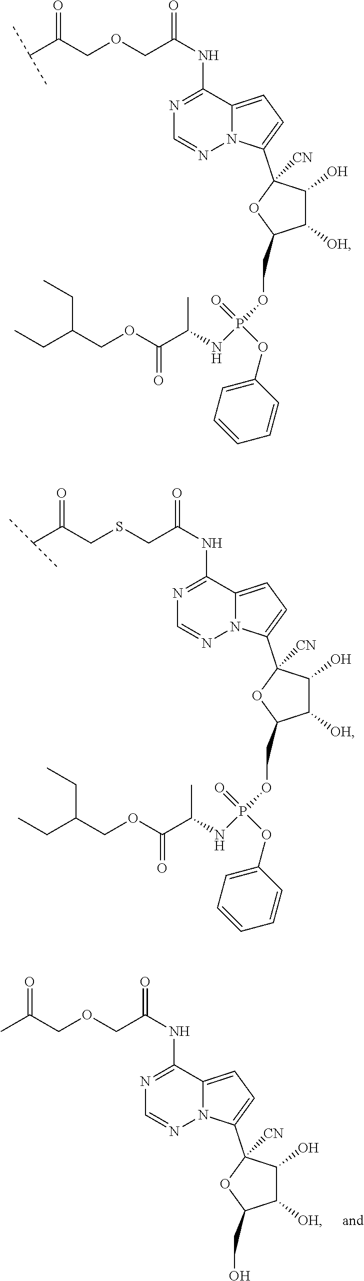

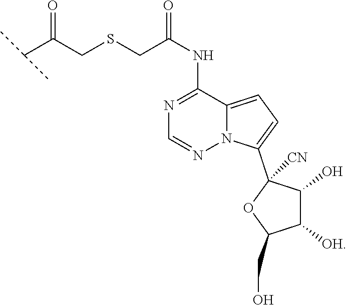

In some embodiments, the dendrimer-drug conjugate used in therapy (e.g. in therapy of a viral infection) has second terminal groups which are each

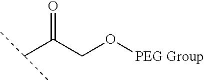

and wherein the PEG group is a methoxy-terminated PEG having a mean molecular weight in the range of from about 500 to 2500 Daltons.

In some embodiments, the dendrimer-drug conjugate used in therapy (e.g. in therapy of a viral infection) has from 26 to 32 first terminal groups, and from 28 to 32 second terminal groups.
In some embodiments, the dendrimer-drug conjugate used in therapy (e.g. in therapy of a viral infection) is

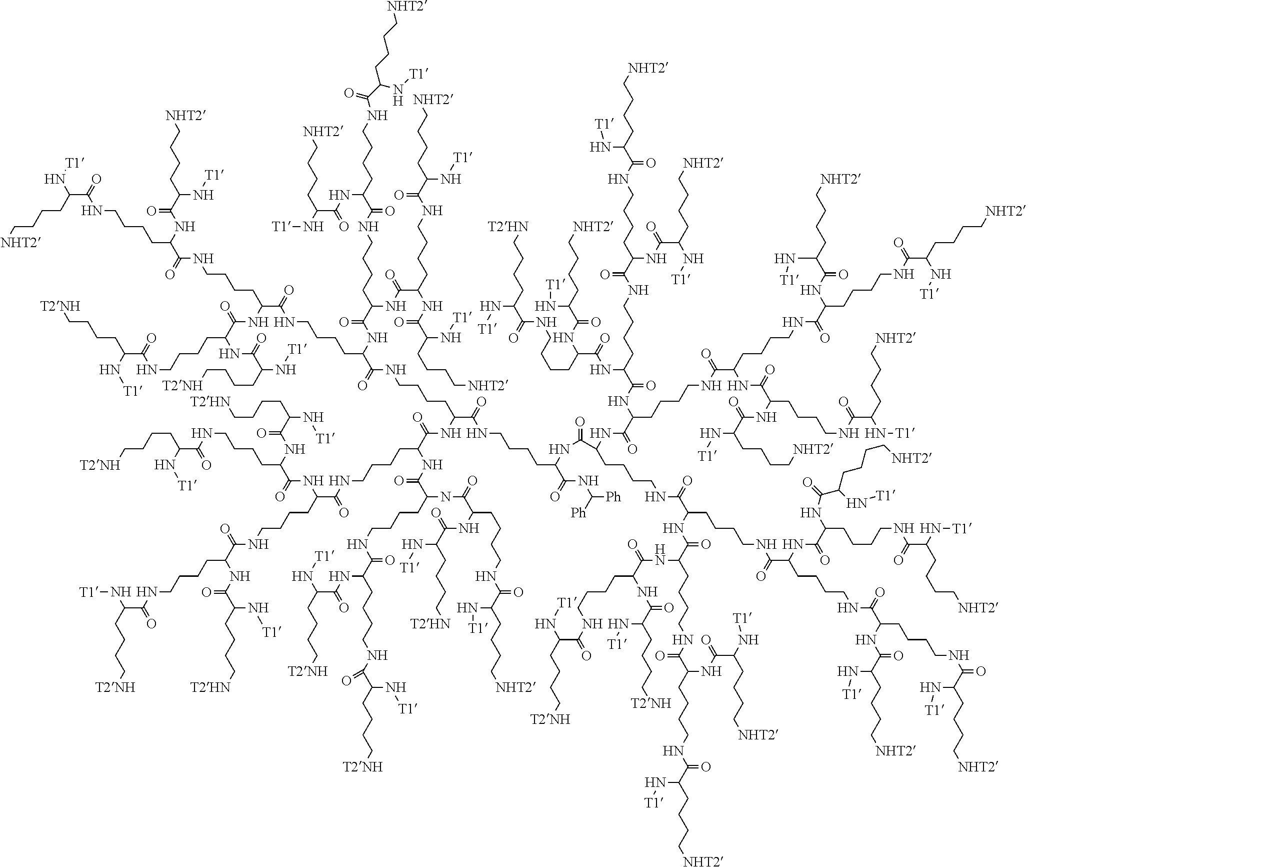

in which T1′ represents a group selected from the group consisting of hydrogen, and

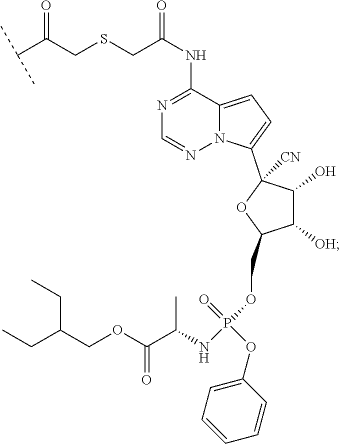

and wherein less than 5 of T1′ are hydrogen; and
T2′ represents a second group which is

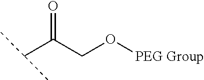

wherein the PEG group is a methoxy-terminated PEG having an average molecular weight in the range of from 500 to 2500 Daltons, or T2′ represents H, and wherein less than 5 of T2′ are H.

### Combinations

Drugs may be co-administered with other drugs in combination therapy, for example during therapy of a viral infection. Accordingly, in some embodiments, the dendrimer-drug conjugate is administered in combination with one or more further therapies, for example one or more further therapeutic agents used for therapy of a viral condition. The dendrimer-drug conjugates and the one or more further therapeutic/pharmaceutically active agents may be administered simultaneously, subsequently or separately. For example, they may be administered as part of the same composition, or by administration of separate compositions.

Accordingly, in some embodiments, there is provided a method of treating a Coronavirus (CoV) infection comprising administering to a subject in need thereof, a combination of a therapeutically effective amount of a dendrimer-drug conjugate or composition, as defined herein, and a therapeutically effective amount of a further therapeutic agent. In some embodiments, the dendrimer-drug conjugate is administered in combination with a further active agent for preventing, treating or reducing the likelihood of infection with a virus. In one embodiment, the virus can infect individuals via the respiratory tract. In one embodiment, the virus can infect individuals via the respiratory tract is selected from: CoV, rhinovirus, influenza virus, syncytial virus, parainfluenza, adenovirus, metapneumovirus and enterovirus. In one embodiment, the virus is a CoV.

In an embodiment, the further active agent is selected from one or more of: an antiviral active agent, a vaccine, an immunomodulator, an antibacterial agent and/or an anti-inflammatory agent.

As used herein the tem “antiviral active agent” refers to a compound that is directly or indirectly effective in specifically interfering with at least one viral action selected from one or more of: virus penetration of a eukaryotic cell, virus replication in a eukaryotic cell, virus assembly, virus release from infected eukaryotic cells, or that is effective in unspecifically inhibiting virus titre increase or in unspecifically reducing a virus titre level in a eukaryotic or mammalian host system. It also refers to an agent that prevents or reduces the likelihood of getting a viral infection.

In an embodiment, the antiviral active agent is selected from an antiviral active agent described in Gordon et al., 2020. In an embodiment, the antiviral active agent is selected from one or more of: carrageenan, GM-CSF, IL-6R, CCR5, S protein of MERS, and drugs including, ribavirin, tilorone, favipiravir, Kaletra (lopinavir/ritonavir), Prezcobix (darunavir/cobicistat), nelfinavir, mycophenolic acid, Galidesivir, Actemra, OYA1, BPI-002, Ifenprodil, APN01, EIDD-2801, baricitinib, camostat mesylate, lycorine, Brilacidin, BX-25, an interferon (e.g. IFNβ), antimalarial chloroquine combined and the antibiotic azithromycin.

Examples of carrageenan are described for example in CA2696009. In one embodiment, the carrageenan is selected from an iota-carrageenan, kappa-carrageenan and a lambda-carrageenan. In one embodiment, the carrageenan is an iota-carrageenan.

In one embodiment, the antibacterial agent is an antibiotic. In an embodiment, the antibiotic is a broad-spectrum antibiotic.

In one embodiment, the immunomodulator is an immunosuppressant, a cytokine inhibitor, an antibody, or an immunostimulant. The immunomodulator may suppress inflammation and/or immune activation (e.g., cell proliferation and homing to tissues) of airways.

The macromolecules or salts thereof may also be used in combination with nonsteroidal anti-inflammatory drug (NSAID). For example, the NSAID may be used to treat the symptoms of a CoV infection, whilst the macromolecule or salt thereof may be used to prevent transmission of the virus to another individual.

As discussed above, the one or more further pharmaceutically active agents may be for preventing, treating or reducing the likelihood of infection with a virus, such as a Coronavirus (CoV) infection. Examples of further therapeutic agents used for therapy of a viral condition include vaccines, plasma therapy, steroids, anti-inflammatory drugs, antipyretic drugs, kinase inhibitors (e.g., berzosertib, imatinib, baricitinib), angiotensin-II receptor blockers, cytokine-blocking monoclonal antibodies (e.g., anakinra, tocilizumab, sarilumab), antiretrovirals (e.g., lopinavir/ritonavir, emtricitabine/tenofovir), and other small molecule therapies (e.g., dexamethasone, colchicine, chloroquine/hydroxychloroquine, losartan, simvastatin).

In some embodiments, the further therapeutic agent is a further dendrimeric therapeutic agent, for example astodrimer or its sodium salt (SPL-7013).

As discussed above, the further therapeutic agent may be delivered as part of the same composition, or delivered in a separate composition from the drug-dendrimer conjugate. The further therapeutic agent may be formulated for administration by any suitable route, for example orally, intravenously, subcutaneously, intramuscularly, intranasally, and/or by inhalation.

The drug dendrimer conjugate may also, for example, be administered as part of a treatment regime involving the use of a ventilator, continuous positive airway pressure (CPAP) device, or other breathing aid. Accordingly, in some embodiments, there is provided a method of treating a Coronavirus (CoV) infection comprising administering to a subject in need thereof a therapeutically effective amount of a dendrimer-drug conjugate, or composition as defined herein, as part of a treatment regime comprising use of a ventilator, CPAP device, or other breathing aid.

### Dosage

It will be appreciated that a therapeutically effective amount refers to a dendrimer-drug conjugate being administered in an amount sufficient to alleviate or prevent to some extent one or more of the symptoms of the disorder or condition being treated.

A therapeutically effective amount of dendrimer-drug conjugate may be referred to on the basis of, for example, the amount of dendrimer-drug conjugate administered. Alternatively, it may be determined based on the amount of active agent (drug moiety comprising Remdesivir nucleoside) which the dendrimer-drug conjugate is theoretically capable of delivering, e.g. based on the loading of drug moiety on the dendrimer.

As used herein, the terms “unconjugated” and “released” refer to a drug moiety which has dissociated or been cleaved from a dendrimer. This dissociation or cleaving may occur in vivo following administration of the drug-dendrimer conjugate.

In a Phase 3 clinical trial, Remdesivir was evaluated over 5-day and 10-day dosing durations in hospitalised patients with severe manifestations of Coronavirus infection. Patients were required to have evidence of pneumonia and reduced oxygen levels that did not require mechanical ventilation at the time of study entry. Efficacy and safety results from the study are depicted in the following table:

| | 5-Day | | 10-Day | | Baseline |
|---|---|---|---|---|---|
| | RDV | | RDV | | adjusted |
| Clinical Efficacy Outcomes at Day 14 | n = 200 | | n = 197 | | p-value¹ |
| ≥ 2-point improvement in ordinal scale | 129 | (65) | 107 | (54) | 0.16 |
| Clinical recovery | 129 | (65) | 106 | (54) | 0.17 |
| Discharge | 120 | (60) | 103 | (52) | 0.44 |
| Death | 16 | (8) | 21 | (11) | 0.70 |
| Safety | | | | | |
| Any adverse event (AE) | 141 | (71) | 145 | (74) | 0.86 |
| Grade ≥ 3 study drug-related AE | 8 | (4) | 10 | (5) | 0.65 |
| Study drug-related serious adverse event | 3 | (2) | 4 | (2) | 0.73 |
| (SAE) | | | | | |
| AE leading to discontinuation | 9 | (5) | 20 | (10) | 0.07 |

¹Adjusted for baseline clinical status
(https://www.gilead.com/news-and-press/press-room/press-releases/2020/4/gilead-announces-results-from-phase-3-trial-of-investigational-antiviral-Remdesivir-in-patients-with-severe-covid-19)

The study demonstrated that the time to clinical improvement for 50% of patients was 10 days in the 5-day treatment group, and 11 days in the 10-day treatment group. More than half of the patients in both treatment groups were discharged from hospital by day 14.

In adult patients (i.e., patients ≥40 kg body weight) requiring invasive mechanical ventilation, the dosage of Remdesivir was a single loading dose of 200 mg, infused intravenously over 30 to 120 minutes on day 1, followed by once-daily maintenance doses of 100 mg infused intravenously over 30 to 120 minutes for days 2 through 10. Alternatively, for adult patients (i.e., patients ≥40 kg body weight) not requiring invasive mechanical ventilation, the dosage of Remdesivir was a single loading dose of 200 mg, infused intravenously over 30 to 120 minutes on day 1, followed by once-daily maintenance doses of 100 mg infused intravenously over 30 to 120 minutes for days 2 through 5. For paediatric patients (i.e., patients ≤40 kg body weight), a body weight-based dosing regimen of one loading dose of Remdesivir at 5 mg/kg intravenous (infused over 30 to 120 minutes) on day 1, followed by Remdesivir at 2.5 mg/kg IV (infused over 30 to 120 minutes) once daily for 9 days (for paediatric patients requiring invasive mechanical ventilation) or 4 days (for patients not requiring invasive mechanical ventilation). Where the patient did not show clinical improvement following 5 days, the duration of treatment may be extended for an additional 5 days. These dosage regimens, in both adults and paediatric patients, were utilised to maintain Remdesivir exposure for the duration of treatment.

Typically, the dendrimer-drug conjugate will provide therapeutic levels of drug moiety comprising Remdesivir nucleoside for prolonged periods, and so can be administered less frequently than Remdesivir. For example, it may be administered once every two days, or once every three days, or once every five days, or once every six days, or once every seven days, or once every eight days, or once every nine days, or once every ten days, or once every two weeks, or once every three weeks, or one every four weeks, or once per month. In some embodiments, a single dose of dendrimer-drug conjugate provides a therapeutically effective amount of the drug moiety comprising Remdesivir nucleoside over a period of at least two days, at least three days, at least four days, at least five days, at least six days, at least seven days, at least eight days, at least nine days, at least ten days, or at least 14 days, or at least 30 days. In some embodiments a single dose of dendrimer-drug conjugate provides a therapeutically effective amount of dendrimer over a period of about two days, about three days, about four days, about five days, about six days, about seven days, about eight days, about nine days, about ten days, about 14 days, or about 30 days.

The use of the conjugates of the present disclosure, which provide for controlled release of drug moiety comprising Remdesivir nucleoside in vivo, allows for administration of a large quantity of conjugated drug moiety in a single dose, which is then released gradually over time.

In some embodiments, the course of therapy of the dendrimer-drug conjugate, or pharmaceutical composition comprising the conjugate, is no more than five doses, no more than four doses, no more than three doses, no more than two doses, or is a single dose.

In some embodiments, the conjugate releases an amount per day in moles of drug moiety comprising Remdesivir nucleoside which is equivalent to the amount in moles of Remdesivir in the range of from 1 mg to 200 mg per day.

In some embodiments, the conjugate releases an amount per day in moles of drug moiety comprising Remdesivir nucleoside which is about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, or about 250 mg per day.

In some embodiments, the dendrimer-drug conjugate is administered to an adult patient, and the conjugate releases an amount per day in moles of drug moiety comprising Remdesivir nucleoside which is equivalent to the amount in moles of Remdesivir nucleoside provided by administration of an amount of Remdesivir in the range of from 100 mg to 200 mg per day (e.g. equivalent to 100 mg per day, or 200 mg per day).

In some embodiments, the dendrimer-drug conjugate is administered to a patient having ≥40 kg body weight, and the conjugate releases an amount per day in moles of drug moiety comprising Remdesivir nucleoside which is equivalent to the amount in moles of Remdesivir nucleoside provided by administration of an amount of Remdesivir in the range of from 100 mg to 200 mg per day.

In some embodiments, the conjugate releases an amount per day in moles of drug moiety comprising Remdesivir nucleoside which is equivalent to the amount of Remdesivir nucleoside provided by administration of an amount of Remdesivir in the range of from 2.5 to 5 mg/kg subject per day (e.g. equivalent to administering about 2.5 mg/kg per day, or 5 mg/kg per day).

In some embodiments the dendrimer-drug conjugate is administered to a patient having <40 kg body weight, and the conjugate releases an amount per day in moles of drug moiety comprising Remdesivir nucleoside which is equivalent to the amount of Remdesivir nucleoside provided by administration of an amount of Remdesivir in the range of from 2.5 to 5 mg/kg subject per day (e.g. equivalent to administering about 2.5 mg/kg per day, or 5 mg/kg per day).

In some embodiments, the amount of dendrimer-drug conjugate administered is sufficient to deliver between 50 mg and 100 mg of drug moiety comprising Remdesivir nucleoside per day.

In some embodiments, the amount of dendrimer-drug conjugate administered is sufficient to deliver an amount of drug moiety comprising Remdesivir nucleoside per day (i.e., the amount of Remdesivir nucleoside released from the dendrimer) which is equivalent to administering between about 5 mg and 200 mg, or between about 5 mg and 100 mg, or between about 10 mg and 50 mg of Remdesivir per day.

In some embodiments, the amount of dendrimer-drug conjugate administered is sufficient to deliver about 5 mg, about 10 mg, about 15 mg, about 20 mg, about 25 mg, about 30 mg, about 35 mg, about 40 mg, about 45 mg, or about 50 mg of drug moiety comprising Remdesivir nucleoside per day. In some embodiments, the amount of dendrimer-drug conjugate administered is sufficient to deliver about 50 mg, about 75 mg, about 100 mg, about 125 mg, about 150 mg, about 175 mg, or about 200 mg of drug moiety comprising Remdesivir nucleoside per day.

The present disclosure provides methods which provide for rapid attainment of therapeutically effective concentrations of drug moiety comprising Remdesivir nucleoside in vivo, and for prolonged exposure to such therapeutically effective concentrations. In some embodiments, this may be achieved by use of a dendrimer-drug conjugate containing drug moieties comprising Remdesivir nucleoside linked to the building units by two different linkers, one providing for faster release of drug moiety, to allow for rapid attainment of therapeutically effective concentrations of drug moiety comprising Remdesivir nucleoside, and the other providing for slower release of drug moiety to allow for slower release of drug moiety comprising Remdesivir nucleoside to allow the in vivo concentration of drug moiety comprising Remdesivir nucleoside to be maintained at a therapeutically effective level. Accordingly, in some embodiments, the method comprises administering a dendrimer-drug conjugate having drug moieties comprising Remdesivir nucleoside conjugated via two different linker groups having differential release rates.

As an alternative, two different dendrimer-drug conjugates according to the present disclosure, may be administered, each containing a different linker group providing for differential release rate of drug moiety comprising Remdesivir nucleoside. Accordingly, in some embodiments, the methods comprise administration of two dendrimer-drug conjugates as described herein, each having a drug moiety comprising a Remdesivir nucleoside conjugated via a different linker, or of a composition or compositions comprising the conjugates, to the subject. The conjugates may be administered simultaneously, sequentially, or separately. As another alternative, a dendrimer-drug conjugate according to the present disclosure, may be administered, containing more than one different linker group, providing for differential release rates of drug moiety. Accordingly, in some embodiments, the methods comprise administration of a dendrimer-drug conjugate as described herein, having drug moieties comprising Remdesivir nucleoside conjugated via two different linker groups, or of a composition or compositions comprising the conjugate, to the subject. As another alternative, a dendrimer-drug conjugate according to the present disclosure, may be administered, containing drug moieties comprising Remdesivir moiety conjugated to the linker at different conjugation sites, providing for differential release rates of drug moiety comprising Remdesivir nucleoside. Accordingly, in some embodiments, the methods comprise administration of a dendrimer-drug conjugate as described herein, having drug moieties comprising a Remdesivir nucleoside conjugated via different conjugation sites, or of a composition or compositions comprising the conjugate, to the subject.

As a further alternative, a dose of Remdesivir itself may be administered on day 1 (e.g. 200 mg Remdesivir), followed by administration of a dose of drug-dendrimer conjugate which comprises drug moiety comprising Remdesivir nucleosides conjugated by a single type of linker, which releases drug moiety so as to maintain a therapeutically effective level. Thus, in some embodiments, the methods comprise administration of a loading dose of Remdesivir or a composition comprising Remdesivir to a subject, followed by administration of a dendrimer-drug conjugate or a pharmaceutical composition comprising the conjugate, to the subject. In some embodiments, the aforementioned administration of Remdesivir or composition containing Remdesivir comprises delivering 100 mg to 200 mg Remdesivir IV. In some embodiments, less than 200 mg, less than 150 mg, or less than 100 mg is delivered.

In some embodiments, where two different linkers are utilised, the first is selected from the group consisting of

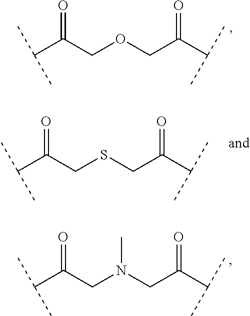

and the second is

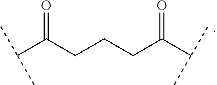

In some embodiments, the dendrimer-drug conjugate or pharmaceutical composition is administered as a fast infusion or as a bolus. In some embodiments, the infusion time is over a period of less than or equal to about 120 minutes, 90 minutes, 60 minutes, 30 minutes, or 20 minutes, for example it may be administered over a period of about 120 minutes, 90 minutes, 60 minutes, 30 minutes, 20 minutes, 15 minutes or 10 minutes. In some embodiments, the dendrimer-drug conjugate or pharmaceutical composition is administered as a fast infusion over a period of 120 minutes. In some embodiments, the dendrimer-drug conjugate or pharmaceutical composition is administered as a fast infusion over a period of 90 minutes. In some embodiments, the dendrimer-drug conjugate or pharmaceutical composition is administered as a fast infusion over a period of 60 minutes. In some embodiments, the dendrimer-drug conjugate or pharmaceutical composition is administered as a fast infusion over a period of 30 minutes.

In some embodiments, the dendrimer-drug conjugate or pharmaceutical composition is administered to the respiratory tract. As used herein, the term “respiratory tract” refers to the passage formed by the mouth, nose, throat, and lungs, through which air passes during breathing. A person skilled in the art will appreciate that the lower respiratory tract comprises one or more of the trachea, primary bronchi, and lungs. In one embodiment, the dendrimer-drug conjugate is administered to the mucosa of the respiratory tract. In one embodiment, the dendrimer-drug conjugate is administered to the mucosa of the trachea. In one embodiment, the dendrimer-drug conjugate is administered to the mucosa of the bronchi. In one embodiment, the dendrimer-drug conjugate is administered to the mucosa of the lungs. In some embodiments, the dendrimer-drug conjugate is administered to the oral or nasal mucosa. In one embodiment, the dendrimer-drug conjugate is administered to the oral mucosa. In one embodiment, the dendrimer-drug conjugate is administered to the nasal mucosa.

The lung is known to be a particularly harsh environment for stability of active agents. Particle size affects the ability of the drug to reach the relevant diseased structures within the lung. On the one hand, the delivery of small molecules directly to the lungs is typically less therapeutically effective as the small molecule will pass through the lung epithelium to be more rapidly cleared into the vascular system. On the other hand, the delivery of large particles to the lung is hampered by the action of the cilia, which remove large particles that are then excreted via the faeces. Thus, a particular advantage of some embodiments of the dendrimer-drug conjugates described herein is that the dendrimer-drug conjugate is less susceptible to clearance and excretion following direct administration to the lung. In some embodiments, the dendrimer-drug conjugate is retained within the lung for a period of time so as to exert a therapeutic effect (i.e., the dendrimer-drug conjugate is retained in the lung at therapeutic levels for a period of time). In some embodiments, the dendrimer-drug conjugate is retained within the lung at therapeutic levels for at least a day, at least a week, or at least a month.

In some embodiments, the dendrimer-drug conjugate or pharmaceutical composition is delivered to the lung by a nasal or pulmonary route. For example, in some embodiments, the dendrimer-drug conjugate is delivered by inhalation, such as inhalation via the mouth or nose. In one embodiment, the dendrimer-drug conjugate is administered by inhalation via the mouth. In one embodiment, the dendrimer-drug conjugate is administered by inhalation via the nose. In some embodiments, the dendrimer-drug conjugate is delivered by intratracheal (IT) instillation or insufflation. In one embodiment, the dendrimer-drug conjugate is delivered by intratracheal (IT) instillation. In one embodiment, the dendrimer-drug conjugate is administered by insufflation. In some embodiments, the dendrimer-drug conjugate may be formulated as an aerosol formulation, a nebulized formulation, a dry powder or aqueous formulation, or an insufflation formulations. In some embodiments, pharmaceutical compositions may be included in pressurized metered dose inhalers, dry powder inhalers, nebulizers, soft mist inhalers, and the like.

In some embodiments, the dendrimer-drug conjugate may be formulated for intra nasal delivery, such as an aqueous nasal spray formulation or a dry powder nasal spray. Nasal spray formulations may include purified aqueous solutions of the active agent with preservative agents and isotonic agents. Such formulations may be adjusted to a pH and isotonic state compatible with the nasal mucous membranes. In one embodiment, the dendrimer-drug conjugate is formulated for nasal delivery. In one embodiment, the pharmaceutical formulation may be suitable for intra nasal delivery, such as an aqueous nasal spray formulation or a dry powder nasal spray. Nasal spray formulations may include purified aqueous solutions of the active agent with preservative agents and isotonic agents. Such formulations may be adjusted to a pH and isotonic state compatible with the nasal mucous membranes. In some embodiments, the dendrimer-drug conjugate is delivered as a powder, a gel, a liquid, an aerosol or an emulsion. In some embodiments, the pH of the formulation is about 4.5 to about 7.42. In one embodiment, the pH of the formulation is about 5 to about 7. In one embodiment, the pH of the formulation is about 5 to about 6.5. In one embodiment, the pH of the formulation is about 5.5 to about 6.5. In one embodiment, the pH of the formulation is about 7.4.

In some embodiments, the osmolality of the formulation is about 200 to about 700 Osmol/kg. In some embodiments, the osmolality of the formulation is about 300 to about 600 Osmol/kg. In some embodiments, the osmolality of the formulation is about 300 to about 700 Osmol/kg. In some embodiments, the osmolality of the formulation is about 200 to about 400 Osmol/kg. In some embodiments, the osmolality of the formulation is about 280 Osmol/kg. Osmolality regulators include, but are not limited to, NaCl, lysine, CaCl₂, and sodium citrate. Regulators of pH include, but are not limited to, H₂SO₄, NaOH, tromethamine, and HCl.

In some embodiment, the dendrimer-drug conjugate is formulated for delivery to the lung. In one embodiment, the dendrimer-drug conjugate is formulated as a dry powder for lung delivery. In one embodiment, the dendrimer-drug conjugate is formulated as a liquid (i.e., nebulised) for lung delivery.

In some embodiments, the dendrimer-drug conjugate is formulated as a dry powder with particle sizes greater than 0.5 μm and less than 50 μm. In some embodiments, the particle size is between 1 μm and 10 μm. In some embodiments, the particle size is between 1 μm and 5 μm. In some embodiments, the dendrimer-drug conjugate may have a particulate size of less than about 100 nm. In some embodiments, the dendrimer-drug conjugate may have a particle size between about 1 nm and about 10 nm, between about 2 nm and about 8 nm, and between about 3 nm and about 6 nm, as determined by DLS. In some embodiments, the dendrimer-drug conjugates have a mean size of about 5 nm, as determined by DLS (at 1 mg/ml in 10-2M NaCl). In some embodiments, the dendrimer-drug conjugate may have a molecular weight of less than 30 kDa, or between about 10 kDa to about 30 kDa, or between about 10 kDa to about 20 kDa.

In some embodiments, a particle diameter of 1 μm to about 5 μm is suitable for delivery to the lower respiratory tract; from 5 to 10 μm particles deposit mostly in the trachea and bronchi, while >10 μm particles deposit mostly in the nose. Usually, particles less than 10 μm median aerodynamic diameter, can reach the lower airways during nasal breathing. In one embodiment, the mean particle size is from about 0.21 μm to about 200 μm. In one embodiment, the mean particle size is from about 1 μm to about 200 μm. In one embodiment, the mean particle size is from about 1 μm to about 50 μm. In one embodiment, the mean particle size is from about 1 μm to about 20 μm. In one embodiment, the mean particle size is from about 1 μm to about 5 μm. The composition may be a liquid, gel or powder.

As described above, in some embodiments, the linker may be selected to release the drug moiety comprising Remdesivir nucleoside at a therapeutic level over a prolonged period of time. In some embodiments, the release profile of the drug moiety from the dendrimer-drug conjugate may provide longer exposure in the lung (i.e., a longer residual time) compared with delivery of Remdesivir for an equivalent amount of Remdesivir nucleoside. In some embodiments, the drug moiety may reside in the lung at least two times, at least three times, at least four times, at least five times, or at least 10 times longer than free Remdesivir.

For lung delivery, the dose required may be less than by alternative delivery routes. In some embodiments the dose delivered to the lung may be less than the dose required for delivery by the IV route. In some embodiments, the dose of the dendrimer-drug conjugate required to achieve a therapeutic effect when delivered to the lungs may be no more than 90%, no more than 75%, no more than 50%, no more than 25%, or no more than 10% of the dose by weight of the dendrimer-drug conjugate required to achieve a therapeutic effect when delivered by the IV route.

In some embodiments, the dendrimer-drug conjugate is retained within the lung to a significant extent. In some embodiments, the percentage of dendrimer-drug conjugate that reaches the systemic circulation is less than 10%, less than 25%, less than 50%, or less than 70%. Systemic delivery in this context refers to the delivery of the drug pharmaceutically active agent to the blood from the lungs, either directly via absorption into lung capillaries or after absorption into pulmonary lymphatic capillaries.

In some embodiments, administration of the dendrimer-drug conjugate results in clinical improvement in the patient within 14 days, within 13 days, within 12 days, within 11 days, within 10 days, within 9 days, within 8 days, within 7 days, within 6 days, within 5 days, within 4 days, within 3 days, within 2 days, or within 1 day of the first administration of the dendrimer-drug conjugate. As used herein, the term “clinical improvement” will be taken to mean an improvement (i.e., a reduction in severity) of one or more symptoms attributed to the Coronavirus (CoV) infection.

In some embodiments, the administration of a course of dendrimer-drug conjugate results in a median time to recover from the Coronavirus (CoV) infection of less than 14 days, less than 13 days, less than 12 days, less than 11 days, less than 10 days, or less than 9 days from the first administration of the dendrimer-drug conjugate. As used herein, the term “time to recover” will be taken to mean the ceasing of one or more symptoms attributed to the Coronavirus (CoV) infection. In one embodiment, the administration of a course of dendrimer-drug conjugate results in a median time to recover of less than 14 days from the first administration of the dendrimer-drug conjugate. In one embodiment, the administration of a course of dendrimer-drug conjugate results in a median time to recover of less than 13 days from the first administration of the dendrimer-drug conjugate. In one embodiment, the administration of a course of dendrimer-drug conjugate results in a median time to recover of less than 12 days from the first administration of the dendrimer-drug conjugate. In one embodiment, the administration of a course of dendrimer-drug conjugate results in a median time to recover of less than 11 days from the first administration of the dendrimer-drug conjugate. In one embodiment, the administration of a course of dendrimer-drug conjugate results in a median time to recover of less than 10 days from the first administration of the dendrimer-drug conjugate. In one embodiment, the administration of a course of dendrimer-drug conjugate results in a median time to recover of less than 9 days from the first administration of the dendrimer-drug conjugate. In one embodiment, the administration of a course of dendrimer-drug conjugate results in a median time to recover of less than 8 days from the first administration of the dendrimer-drug conjugate. In one embodiment, the administration of a course of dendrimer-drug conjugate results in a median time to recover of less than 7 days from the first administration of the dendrimer-drug conjugate. In one embodiment, the administration of a course of dendrimer-drug conjugate results in a median time to recover of less than 6 days from the first administration of the dendrimer-drug conjugate. In one embodiment, the administration of a course of dendrimer-drug conjugate results in a median time to recover of less than 5 days from the first administration of the dendrimer-drug conjugate.

In some embodiments, the term “time to recover” refers to a patient no longer requiring oxygen support (e.g., mechanical ventilation). In some embodiments, the administration of a course of dendrimer-drug conjugate results in a patient no longer requiring oxygen support within 1 day, 2 days, 3 days, 4 days, 5 days, 6 days, 7 days, 8 days, 9 days, 10 days, 11 days, 12 days, 13 days, 14 days, or 15 days of the first administration of the dendrimer-drug conjugate. In one embodiment, the administration of a course of dendrimer-drug conjugate results in a patient no longer requiring oxygen support within 1 day of the first administration of the dendrimer-drug conjugate. In one embodiment, the administration of a course of dendrimer-drug conjugate results in a patient no longer requiring oxygen support within 2 days of the first administration of the dendrimer-drug conjugate. In one embodiment, the administration of a course of dendrimer-drug conjugate results in a patient no longer requiring oxygen support within 3 days of the first administration of the dendrimer-drug conjugate. In one embodiment, the administration of a course of dendrimer-drug conjugate results in a patient no longer requiring oxygen support within 4 days of the first administration of the dendrimer-drug conjugate. In one embodiment, the administration of a course of dendrimer-drug conjugate results in a patient no longer requiring oxygen support within 5 days of the first administration of the dendrimer-drug conjugate. In one embodiment, the administration of a course of dendrimer-drug conjugate results in a patient no longer requiring oxygen support within 6 days of the first administration of the dendrimer-drug conjugate. In one embodiment, the administration of a course of dendrimer-drug conjugate results in a patient no longer requiring oxygen support within 7 days of the first administration of the dendrimer-drug conjugate. In one embodiment, the administration of a course of dendrimer-drug conjugate results in a patient no longer requiring oxygen support within 8 days of the first administration of the dendrimer-drug conjugate. In one embodiment, the administration of a course of dendrimer-drug conjugate results in a patient no longer requiring oxygen support within 9 days of the first administration of the dendrimer-drug conjugate. In one embodiment, the administration of a course of dendrimer-drug conjugate results in a patient no longer requiring oxygen support within 10 days of the first administration of the dendrimer-drug conjugate. In one embodiment, the administration of a course of dendrimer-drug conjugate results in a patient no longer requiring oxygen support within 11 days of the first administration of the dendrimer-drug conjugate. In one embodiment, the administration of a course of dendrimer-drug conjugate results in a patient no longer requiring oxygen support within 12 days of the first administration of the dendrimer-drug conjugate. In one embodiment, the administration of a course of dendrimer-drug conjugate results in a patient no longer requiring oxygen support within 13 days of the first administration of the dendrimer-drug conjugate. In one embodiment, the administration of a course of dendrimer-drug conjugate results in a patient no longer requiring oxygen support within 14 days of the first administration of the dendrimer-drug conjugate.

In some embodiments, “time to recover” refers to a patient being discharged from hospital care. In some embodiments, the administration of a course of dendrimer-drug conjugate results in a patient being discharged from hospital care within 1 day, 2 days, 3 days, 4 days, 5 days, 6 days, 7 days, 8 days, 9 days, 10 days, 11 days, 12 days, 12 days, 14 days, 15 days, 16 days, 17 days, 18 days, 19 days, 20 days, 21 days, or 28 days of the first administration of the dendrimer-drug conjugate. In one embodiment, the administration of a course of dendrimer-drug conjugate results in a patient being discharged from hospital care within 1 day of the first administration of the dendrimer-drug conjugate. In one embodiment, the administration of a course of dendrimer-drug conjugate results in a patient being discharged from hospital care within 5 days of the first administration of the dendrimer-drug conjugate. In one embodiment, the administration of a course of dendrimer-drug conjugate results in a patient being discharged from hospital care within 7 days of the first administration of the dendrimer-drug conjugate. In one embodiment, the administration of a course of dendrimer-drug conjugate results in a patient being discharged from hospital care within 10 days of the first administration of the dendrimer-drug conjugate. In one embodiment, the administration of a course of dendrimer-drug conjugate results in a patient being discharged from hospital care within 14 days of the first administration of the dendrimer-drug conjugate. In one embodiment, the administration of a course of dendrimer-drug conjugate results in a patient being discharged from hospital care within 21 days of the first administration of the dendrimer-drug conjugate. In one embodiment, the administration of a course of dendrimer-drug conjugate results in a patient being discharged from hospital care within 28 days of the first administration of the dendrimer-drug conjugate.

Administration of the dendrimer-drug conjugate to a subject suffering from Coronavirus (CoV) infection may also result in a decreased morality rate of the subject. In some embodiments, administration of the dendrimer-drug conjugate to a subject results in a mortality rate of less than 15%, less than 11%, less than 10%, less than 9%, less than 8%, less than 7%, less than 6%, or less than 5%. In one embodiment, administration of the dendrimer-drug conjugate to a subject results in a mortality rate of less than 11%. In one embodiment, administration of the dendrimer-drug conjugate to a subject results in a mortality rate of less than 10%. In one embodiment, administration of the dendrimer-drug conjugate to a subject results in a mortality rate of less than 9%. In one embodiment, administration of the dendrimer-drug conjugate to a subject results in a mortality rate of less than 8%. In one embodiment, administration of the dendrimer-drug conjugate to a subject results in a mortality rate of less than 7%. In one embodiment, administration of the dendrimer-drug conjugate to a subject results in a mortality rate of less than 6%. In one embodiment, administration of the dendrimer-drug conjugate to a subject results in a mortality rate of less than 5%.

In some embodiments, the dendrimer-drug conjugate is administered to a patient receiving oxygen support (e.g., ventilation). In some embodiments, the dendrimer-drug conjugate is administered to a patient not yet receiving oxygen support (e.g., ventilation), though may be progressing toward requiring oxygen support. In some embodiments, the dendrimer-drug conjugate is administered to a patient demonstrating pneumonia-like symptoms.

Coronavirus (CoV) infection may be defined as mild, moderate, serious and critical/severe/extreme. This definition typically refers to the severity of the symptoms exhibited by the person suffering from the Coronavirus (CoV) infection. In some embodiments, the dendrimer-drug conjugate is administered to a patient suffering from a mild Coronavirus (CoV) infection. In some embodiments, the dendrimer-drug conjugate is administered to a patient suffering from a moderate Coronavirus (CoV) infection. In some embodiments, the dendrimer-drug conjugate is administered to a patient suffering from a serious Coronavirus (CoV) infection. In some embodiments, the dendrimer-drug conjugate is administered to a patient suffering from a critical Coronavirus (CoV) infection. In some embodiments, the dendrimer-drug conjugate is administered to a patient suffering from a severe Coronavirus (CoV) infection. In some embodiments, the dendrimer-drug conjugate is administered to a patient suffering from an extreme Coronavirus (CoV) infection.

Coronavirus (CoV) infection may affect both male and female patients, however, it is initially thought to exhibit more severe symptoms in male sufferers. In one embodiment, the dendrimer-drug conjugate is administered to a female patient suffering from Coronavirus (CoV) infection. In one embodiment, the dendrimer-drug conjugate is administered to a male patient suffering from a Coronavirus (CoV) infection.

A patient may also test positive for Coronavirus infection, yet not show any symptoms (i.e., is asymptomatic). In some embodiments, the dendrimer-drug conjugate is administered to a patient suffering from an asymptomatic Coronavirus (CoV) infection.

### Pharmacokinetics

The pharmacokinetic properties of Remdesivir and the predominant circulating metabolite GS-441524 have been evaluated in healthy adult subjects. Following intravenous administration of Remdesivir adult dosage regimen, peak plasma concentration was observed at end of infusion, regardless of dose level, and declined rapidly thereafter with a half-life of approximately 1 hour. Peak plasma concentrations of GS-441524 were observed at 1.5 to 2.0 hours post start of a 30 minutes infusion of about 150 ng/ml (John Hopkins medicine ABX guide). Plasma concentration for GS-441524 in the 24 hours following 100 mg infusion ranged from about 100 to 300 ng/ml in 2 patients (Yan and Muller, 2020).

In some embodiments the dendrimer-drug conjugate produces a plasma concentration of released/unconjugated Remdesivir of greater than 50 ng/ml for at least 6 hours, 12 hours, 24 hours, 48 hours, or 96 hours, 120 hours or 168 hours. In some embodiments, the dendrimer-drug conjugate produces a plasma concentration of released/unconjugated Remdesivir of greater than 100 ng/ml for at least 6 hours, 12 hours, 24 hours, or 48 hours, 96 hours or 168 hours. In some embodiments the dendrimer-drug conjugate produces a plasma concentration of drug moiety comprising Remdesivir nucleoside of greater than 50 ng/ml for at least 12 hours, 24 hours, 48 hours, 96 hours, 120 hours or 168 hours. In some embodiments the dendrimer-drug conjugate produces a plasma concentration of drug moiety comprising Remdesivir nucleoside of greater than 100 ng/ml for at least 12 hours, 24 hours, 48 hours, 96 hours or 168 hours.

Remdesivir is extensively metabolized to the pharmacologically active nucleoside analog triphosphate GS-443902 (formed intracellularly). The metabolic activation pathway involves hydrolysis by esterases, which leads to the formation of the intermediate metabolite, GS-704277. Phosphoramidate cleavage followed by phosphorylation forms the active triphosphate, GS-443902. Dephosphorylation of all phosphorylated metabolites can result in the formation of nucleoside metabolite GS-441524 that itself is not efficiently re-phosphorylated. The human mass balance study also indicates presence of a currently unidentified major metabolite (M27) in plasma.

One study reported that, following an IV infusion of 200 mg Remdesivir to healthy human subjects, the AUC0-24 values were 4.8 μM·h for Remdesivir and 7.7 μM·h for the nucleoside metabolite GS-441524 (Study GS-US-399-5505) (EMA Summary on compassionate use, Remdesivir Gilead).

Renal clearance is understood to be the major elimination pathway for GS-441524. The median terminal half-lives of Remdesivir and GS-441524 have been reported as approximately 1 and 27 hours, respectively (European Summary of Product Characteristics for Remdesivir).

As discussed above, Remdesivir has a comparatively short half-life and is rapidly metabolised to other active metabolites. The dendrimer-drug conjugates of the present disclosure release drug moiety gradually over time, and thereby achieve a sustained pharmacokinetic profile for unconjugated or released drug. This sustained pharmacokinetic profile indicates that the drug may be present in vivo at therapeutically effective levels for longer periods of time. It will be appreciated that exposure to the drug for a longer period of time is desirable as it may prolong the therapeutic effect of the drug and allow for reduced frequency of dosing. In some embodiments, administration of the dendrimer-drug conjugate provides a therapeutically effective plasma concentration of drug moiety for a longer period of time, in comparison to administration of an equivalent dose of free Remdesivir nucleoside. The route of delivery may impact the pharmacokinetic profile with subcutaneous delivery providing delayed T max, increased t112, and lower Cmax and AUC compared to intravenous administration.

In some embodiments, the dendrimers of the present disclosure provides at least 2 times, at least 2.5 times, at least 3 times, at least 3.5 times, at least 4 times, at least 5 times, or at least 10 times the tin, of Remdesivir, in comparison to administration of an equivalent dose of Remdesivir. The half-life of a drug is the time it takes for the blood plasma concentration of the drug to halve. It will be appreciated that an increased (i.e., longer) half-life may be desirable since it results in exposure to therapeutically effective concentrations of drug for a longer period of time. It may also result in the need for less frequent dosing. In some embodiments, administration of the dendrimer results in a pharmacokinetic profile of free Remdesivir having a t_{1/2} of at least about 2 hours, at least about 5 hours, at least about 10 hours, at least about 20 hours, at least about 25 hours, at least about 30 hours, at least about 40 hours, or at least about 50 hours.

In some embodiments, administration of the dendrimer provides a pharmacokinetic (PK) profile of Remdesivir having a Tₘₐₓ of at least about 1 hour, at least about 2 hours, least about 10 hours, at least about 20 hours, or at least about 30 hours.

In some embodiments, the observed pharmacokinetic profile for GS-441524 released from the dendrimer has an increased half-life (t_{1/2}) in comparison to the direct administration of an equivalent dose of Remdesivir. In some embodiments, the dendrimer provides an increased terminal half-life (t_{1/2}) of GS-441524 in comparison to the direct administration of an equivalent dose of Remdesivir. In some embodiments, the dendrimers of the present disclosure provides at least 2 times, at least 2.5 times, at least 3 times, at least 3.5 times, at least 4 times, or at least 5 times, the tin, of GS-441524, in comparison to administration of an equivalent dose of Remdesivir. In some embodiments, administration of the dendrimer results in a pharmacokinetic profile of GS-441524 having a t_{1/2} of least about 15 hours, at least about 20 hours, at least about 30 hours, at least about 40 hours, at least about 50 hours, or at least about 60 hours.

In some embodiments, administration of the dendrimer provides a pharmacokinetic profile of GS-441524 having a Tₘₐₓ of at least about 5 hours, at least about 10 hours, or at least about 20 hours.

In some embodiments, administration of the dendrimer-drug conjugate may provide a lower maximal concentration (Cmax) of unconjugated/released drug in comparison to direct administration of an equivalent dose of free drug. The maximal concentration (Cmax) of drug is the maximum (or peak) serum concentration that a drug achieves in a specified compartment or test area of the body after the drug has been administered and before the administration of a second dose. It will be appreciated that, whilst it is important to be able to dose a pharmaceutical agent at a level sufficient to achieve therapeutic concentration levels, if the maximum concentration levels reached are high, the risk of encountering certain off-target effects, side-effects and toxicity increase. This is particularly an issue for compounds which have a short half-life, since in such cases, in order to provide therapeutically effective levels of the active agent for a prolonged period of time, it may be necessary to increase the dose and thus the Cmax such that the likelihood of side effects increases. Accordingly, it is highly desirable to be able to deliver a pharmaceutically active agent in a form which provides therapeutically effective levels for a sustained period of time, whilst at the same time avoiding dosing at levels that achieve very high maximum concentrations (Cmax) in vivo.

In some embodiments, the dendrimer-drug conjugate may have a lower maximal concentration (Cmax) of unconjugated/released drug moiety comprising Remdesivir nucleoside in comparison to the direct administration of an equivalent dose of free Remdesivir nucleoside (e.g. Remdesivir). In some embodiments, the dendrimer-drug conjugate may have a lower maximal concentration (Cmax) of released/unconjugated drug moiety in comparison to the direct administration of an equivalent dose of free Remdesivir when used in a method of treatment, for example, in the treatment of a viral infection, such as a Coronavirus (CoV) infection.

In some embodiments, the dendrimer-drug conjugate may have a lower maximal concentration (Cmax) of unconjugated/released drug moiety comprising Remdesivir nucleoside in comparison to the direct administration of a dose of 200 mg Remdesivir nucleoside. In some embodiments, the dendrimer-drug conjugate may have a lower maximal concentration (Cmax) of released/unconjugated drug moiety in comparison to the direct administration of a dose of 200 mg Remdesivir, when used in a method of treatment, for example, in the treatment of a viral infection, such as a Coronavirus (CoV) infection.

In some embodiments, following administration of a dose of drug-dendrimer conjugate, the Cmax of released drug moiety comprising Remdesivir nucleoside (e.g. Remdesivir) is no more than one tenth, no more than one eighth, no more than one sixth, no more than one quarter, no more than one third, no more than one half, or no more than three quarters of the Cmax of free Remdesivir nucleoside (e.g. Remdesivir) following administration of an equivalent dose of Remdesivir.

Cmax has been reported as 5440 ng/ml for Remdesivir and 152 ng/ml for the metabolite GS-441524 after 200 mg dose (John Hopkins).

In some embodiments, administration of the dendrimer provides plasma Cmax less than about 2000 ng/mL, less than about 1000 ng/mL, or less than about 500 ng/mL of Remdesivir.

In some embodiments, administration of the dendrimer provides plasma Cmax less less than about 200 ng/mL, less than about 100 ng/mL, or less than about 50 ng/mL of GS-441524.

In some embodiments, administration of the dendrimer provides plasma levels of Remdesivir of greater than 10 ng/mL for at least 1, 2, 3, 4, 5, 6, or 7 days. In one example, administration of the dendrimer provides plasma levels of Remdesivir of greater than 10 ng/mL for at least 5 days. In some embodiments, administration of the dendrimer provides plasma levels of Remdesivir of greater than 100 ng/mL for at least 12 hours, 24 hours, 36 hours, 2 days, or 3 days. In one example, administration of the dendrimer provides plasma levels of Remdesivir of greater than 100 ng/mL for at least 3 days.

In some embodiments, administration of the dendrimer provides plasma levels of GS-441524 of greater than 10 ng/mL for at least 12 hours, 24 hours, 36 hours, 2 days, 3 days, 4 days, or 5 days. In one example, administration of the dendrimer provides plasma levels of GS-441524 of greater than 10 ng/mL for at least 2 days. In some embodiments, administration of the dendrimer provides plasma levels of GS-441524 of greater than 5 ng/mL for at least 1, 2, 3, 4, 5, 6, or 7 days. In one example, administration of the dendrimer provides plasma levels of GS-441524 of greater than 5 ng/mL for at least 5 days.

In some embodiments the dendrimer-drug conjugate produces a Cmax of released unconjugated drug moiety (e.g. Remdesivir and/or other drug moiety comprising Remdesivir nucleoside) of less than 5000 ng/ml, less than 4000 ng/ml, less than 3000 ng/ml, less than 2000 ng/ml. In some embodiments the dendrimer-drug conjugate produces a Cmax of drug moiety (e.g. Remdesivir and/or other drug moiety comprising Remdesivir nucleoside) of less than 500 ng/ml, less than 400 ng/ml, less than 300 ng/ml, less than 200 ng/ml or less than 100 ng/ml.

AUC is the area under the curve in a plot of drug concentration in blood plasma versus time. The AUC represents the total drug exposure over time. It will be appreciated that the AUC is normally proportional to the total amount of drug delivered to the body.

It will be appreciated that, following administration of the dendrimer-drug conjugate, and as some of the drug moiety is released from the dendrimer, there is both unbound drug moiety present in the body, and drug moiety comprising Remdesivir nucleoside present which is still bound to dendrimer.

In some embodiments, administration of the dendrimer-drug conjugate may provide greater AUC of total drug moiety comprising Remdesivir nucleoside (i.e. both dendrimer-bound Remdesivir nucleoside and released Remdesivir nucleoside), in comparison to direct administration of an equivalent dose of free Remdesivir.

In some embodiments, administration of the dendrimer-drug conjugate may provide equivalent or greater AUC of unconjugated/released drug moiety comprising Remdesivir nucleoside in comparison to the direct administration of an equivalent dose of free Remdesivir.

In some embodiments, administration of the dendrimer-drug conjugate may provide at least 1.1 times, at least 1.2 times, at least 1.3 times, at least 1.4 times, at least 1.5 times, at least 1.6 times, at least 1.7 times, at least 1.8 times, at least 1.9 times, at least 2 times, at least 2.5 times, at least 3 times, at least 3.5 times, or at least 4 times, at least 10 times, or at least 20 times, at least 100 times the AUCinf of unconjugated/released drug moiety comprising Remdesivir nucleoside in comparison to the direct administration of an equivalent dose of free Remdesivir nucleoside.

In some embodiments, administration of the dendrimer provides at least 500 ng/h/mL, at least 1000 ng/h/mL, at least 5000 ng/h/mL, at least 10,000 ng/h/mL, at least 50,000 ng/h/mL, or at least 100,000 ng/h/mL of Remdesivir.

As discussed above, following IV administration of 200 mg Remdesivir to humans, the AUC₀₋₂₄ values were 4.8 μM·h. In some embodiments, following administration of a dose of drug-dendrimer conjugate containing equivalent drug moiety comprising Remdesivir nucleoside to 200 mg Remdesivir, the AUC₀₋₂₄ of released Remdesivir nucleoside (e.g. Remdesivir) is at least 6 μM·h, at least 8 μM·h, at least 10 μM·h, at least 15 μM·h, or at least 20 μM·h.

In some embodiments, administration of the dendrimer-drug conjugate may provide equivalent or greater AUC of unconjugated/released drug moiety comprising Remdesivir nucleoside in comparison to the direct administration of an equivalent dose of free Remdesivir, when used in a method of treatment, for example, in the treatment of a viral infection, such as a Coronavirus (CoV) infection.

In some embodiments, the dendrimer provides increased therapeutic drug exposure/area under the curve (AUC) of GS-441524 active in comparison to direct administration of an equivalent dose of free Remdesivir (unconjugated). In some embodiments, administration of the dendrimer provides at least 1.5 times, at least 2 times, at least 2.5 times, at least 3 times, at least 3.5 times, at least 4 times, at least 5 times, the therapeutic drug exposure (AUC) of GS-441524 in comparison to the direct administration of an equivalent dose of free Remdesivir (unconjugated). In some embodiments, administration of the dendrimer provides at least 500 ng/h/mL, at least 1000 ng/h/mL, at least 2000 ng/h/mL, at least 3000 ng/h/mL, at least 3500 ng/h/mL, at least 4000 ng/h/mL, or at least 5000 ng/h/mL of GS-441524.

It will be appreciated that any one or more of the above pharmacokinetic properties may provide better clinical efficacy in comparison to the direct administration of the free drug. In some embodiments, administration of the dendrimer-drug conjugate provides better efficacy of the drug, in comparison to the direct administration of an equivalent dose of the free drug.

### Delivery Devices

In one aspect, the present disclosure also provides a device for delivering a nasal or pulmonary formulation comprising a dendrimer-drug conjugate as described herein. The devices as described herein can deliver the dendrimer-drug conjugate to the upper and/or lower respiratory tract. In an embodiment, the device can delivery one or more doses. In an embodiment, the device is reusable.

In an embodiment, the device is a nasal delivery device. In an embodiment, the device is an oral delivery device (e.g. an asthma puffer). In an embodiment, the nasal delivery device is selected from a spray, inhaler, nebulizer or nasal wash.

In one embodiment, the device is a nasal spray. In an embodiment, the nasal spray is a pump spray. Nasal spray pumps are displacement pumps and when actuating the pump by pressing the actuator towards the bottle, a piston moves downward in the metering chamber. A valve mechanism at the bottom of the metering chamber will prevent backflow into the dip tube. So the downward movement of the piston will create pressure within the metering chamber which forces the air (before priming) or the liquid outwards through the actuator and generates the spray. When the actuation pressure is removed, a spring will force the piston and actuator to return to its initial position. The metering chamber ensures the right dosing and an open swirling chamber in the tip of the actuator will aerosolize the metered dose. In these pumps no measures are taken to prevent microbial contamination when in use, thus the formulation often will contain preservatives, in most cases benzalkonium chloride (BAC) or parabens. In some embodiments, the device uses silver as a preservative. In an embodiment, the device uses a silver wire in the tip of the actuator, a silver coated spring and ball. Such systems are able to keep microorganisms from contaminating the formulation between long dosing intervals. Another approach is to use tip seal technology to prevent backflow into the device. In some embodiments, the total dose delivered is about 25 to about 200 μL per dose. In some embodiments, the total dose delivered is about 50 to about 150 μL per dose. In one embodiment, the total dose is about 150 μL per dose. In one embodiment, the total dose is about 100 μl. In one embodiment, the total dose is about 50 In some embodiments, the nasal spray delivers an average particle size of about 10 to about 200 μm. In some embodiments, the nasal spray delivers an average particle size of about 20 to about 180 μm. In some embodiments, the nasal spray delivers an average particle size of about 40 to about 160 μm. In some embodiments, the nasal spray delivers an average particle size of about 60 to about 110 μm. In one embodiment, the dose is delivered to each nostril.

In an embodiment, the device is an oral delivery device. A person skilled in the art will appreciated that the oral delivery device may be a pulmonary oral delivery device, for example as described in Ibrahim et al (2015) or Chandel et al (2019). In an embodiment, the oral delivery device is selected from a spray, inhaler, nebulizer or oral wash. In an embodiment, the device can deliver one or more doses. In an embodiment, the device is reusable.

In an embodiment, the oral device is an oral spray. In an embodiment, the oral spray is a pump spray.

In an embodiment, the device is an inhaler. In an embodiment, the inhaler is a metered-dose inhaler. In an embodiment, the inhaler is a multi-dose inhaler. In an embodiment, the inhaler is a dry powder inhaler. Examples of inhalers can be found in Chandel et al (2019).

In some embodiments, the total dose delivered by the inhaler is about 5 to about 150 μL per dose. In some embodiments, the total dose delivered is about 10 to about 110 μL per dose. In one embodiment, the total dose is about 20 μL to about 100 μL per dose. In one embodiment, the total dose is about 40 μL to about 80 μL per dose.

In an embodiment, the inhaler delivers an average particle size of about 0.01 to about 7 μm. In an embodiment, the nebuliser delivers an average particle size of about 0.01 to about 5 μm. In an embodiment, the nebuliser delivers an average particle size of about 0.5 to about 5 μm. In an embodiment, the nebuliser delivers an average particle size of about 1 to about 5 μm. In an embodiment, the nebuliser delivers an average particle size of about 2 to about 4 μm.

In an embodiment, the nebuliser is a jet nebuliser. In an embodiment the nebuliser is an ultrasonic nebuliser. In an embodiment, the nebuliser is a vibrating mesh nebuliser. In an embodiment, the nebuliser is a breath actuated nebuliser. In an embodiment the nebuliser is a breath enhanced nebuliser. In an embodiment, the nebuliser is selected from a: Spiriva Respimat®, the AERx® Pulmonary Drug Delivery System, AeroEclipse® II BAN (Monaghan Medical Corporation), CompAIR™ NE-C801 (OMRON Healthcare Europe BV), I-neb AAD System (Koninklijke Philips NV), Micro Air® NE-U22 (OMRON Healthcare Europe BV), PARI LC® Plus (PARI international), PART eFlow® rapid (PARI international) and a AKITA® Inhalation System (Activaero)

In some embodiments, the total dose delivered by the nebuliser is about 5 to about 150 μL per dose. In some embodiments, the total dose delivered is about 10 to about 110 μL per dose. In one embodiment, the total dose is about 20 μL to about 100 μL per dose. In one embodiment, the total dose is about 40 μL to about 80 μL per dose.

In an embodiment, the nebuliser delivers an average particle size of about 0.01 to about 7 μm. In an embodiment, the nebuliser delivers an average particle size of about 0.01 to about 5 μm. In an embodiment, the nebuliser delivers an average particle size of about 0.5 to about 5 μm. In an embodiment, the nebuliser delivers an average particle size of about 1 to about 5 μm. In an embodiment, the nebuliser delivers an average particle size of about 2 to about 4 μm.

### Compositions

In some embodiments, the dendrimer-drug conjugate is presented as a composition, e.g. a pharmaceutical composition.

It will be appreciated that there may be some variation in the molecular composition between the dendrimer-drug conjugates present in a given composition, as a result of the nature of the synthetic process for producing the dendrimers-drug conjugates. For example, as discussed above one or more synthetic steps used to produce a dendrimer-drug conjugate may not proceed fully to completion, which may result in the presence of dendrimer-drug conjugates that do not all comprise the same number of first terminal groups or second terminal groups, or which contain incomplete generations of building units.

In some embodiments, there is provided a composition comprising a plurality of dendrimer-drug conjugates or pharmaceutically acceptable salts thereof, wherein the dendrimer-drug conjugates are as defined herein, and as having five generations of building units,
the mean number of first terminal groups per dendrimer-drug conjugate in the composition is in the range of from 26 to 32, and
the mean number of second terminal groups per dendrimer-drug conjugate in the composition is in the range of from 26 to 32. In some embodiments, the mean number of first terminal groups per dendrimer-drug conjugate is in the range of from 28 to 32, and wherein the mean number of second terminal groups per dendrimer-drug conjugate is in the range of from 28 to 32. In some embodiments, the mean number of first terminal groups per dendrimer-drug conjugate is in the range of from 29 to 32, and wherein the mean number of second terminal groups per dendrimer-drug conjugate is in the range of from 29 to 32. In some embodiments, the mean number of first terminal groups per dendrimer-drug conjugate is in the range of from 30 to 32, and wherein the mean number of second terminal groups per dendrimer-drug conjugate is in the range of from 30 to 32.

In some embodiments, at least 50%, at least 60%, at least 70%, at least 80%, at least 90%, or at least 95% of the dendrimer-drug conjugates contain at least 26 first terminal groups. In some embodiments, at least 50%, at least 60%, at least 70%, at least 80%, at least 90%, or at least 95% of the dendrimers contain at least 27 first terminal groups. In some embodiments, at least 50%, at least 60%, at least 70%, at least 80%, at least 90%, or at least 95% of the dendrimers contain at least 28 first terminal groups. In some embodiments, at least 50%, at least 60%, at least 70%, at least 80%, at least 90%, or at least 95% of the dendrimers contain at least 29 first terminal groups. In some embodiments, at least 50%, at least 60%, at least 70%, at least 80%, at least 90%, or at least 95% of the dendrimers contain at least 30 first terminal groups. In some embodiments, at least 50%, at least 60%, at least 70%, at least 80%, at least 90%, or at least 95% of the dendrimers contain at least 31 first terminal groups.

In some embodiments, at least 50%, at least 60%, at least 70%, at least 80%, at least 90%, or at least 95% of the dendrimers contain at least 26 second terminal groups. In some embodiments, at least 50%, at least 60%, at least 70%, at least 80%, at least 90%, or at least 95% of the dendrimers contain at least 27 second terminal groups. In some embodiments, at least 50%, at least 60%, at least 70%, at least 80%, at least 90%, or at least 95% of the dendrimers contain at least 28 second terminal groups. In some embodiments, at least 50%, at least 60%, at least 70%, at least 80%, at least 90%, or at least 95% of the dendrimers contain at least 29 second terminal groups. In some embodiments, at least 50%, at least 60%, at least 70%, at least 80%, at least 90%, or at least 95% of the dendrimers contain at least 30 second terminal groups. In some embodiments, at least 50%, at least 60%, at least 70%, at least 80%, at least 90%, or at least 95% of the dendrimers contain at least 31 second terminal groups.

In some embodiments, at least 50%, at least 60%, at least 70%, at least 80%, at least 90%, or at least 95% of the dendrimers contain at least 26 first terminal groups and at least 26 second terminal groups. In some embodiments, at least 50%, at least 60%, at least 70%, at least 80%, at least 90%, or at least 95% of the dendrimers contain at least 27 first terminal groups and at least 27 second terminal groups. In some embodiments, at least 50%, at least 60%, at least 70%, at least 80%, at least 90%, or at least 95% of the dendrimers contain at least 28 first terminal groups and at least 28 second terminal groups. In some embodiments, at least 50%, at least 60%, at least 70%, at least 80%, at least 90%, or at least 95% of the dendrimers contain at least 29 first terminal groups and at least 29 second terminal groups. In some embodiments, at least 50%, at least 60%, at least 70%, at least 80%, at least 90%, or at least 95% of the dendrimers contain at least 30 first terminal groups and at least 30 second terminal groups. In some embodiments, at least 50%, at least 60%, at least 70%, at least 80%, at least 90%, or at least 95% of the dendrimers contain at least 31 first terminal groups and at least 31 second terminal groups.

In some embodiments, the composition is a pharmaceutical composition, and the composition comprises a pharmaceutically acceptable excipient.

The present disclosure also provides pharmaceutical formulations or compositions, both for veterinary and for human medical use, which comprise the dendrimer-drug conjugates of the present disclosure or a pharmaceutically acceptable salt thereof, with one or more pharmaceutically acceptable carriers, and optionally any other therapeutic ingredients, stabilisers, or the like. The carrier(s) must be pharmaceutically acceptable in the sense of being compatible with the other ingredients of the formulation and not unduly deleterious to the recipient thereof. The compositions may further include diluents, buffers, citrate, trehalose, binders, disintegrants, thickeners, lubricants, preservatives (including antioxidants), inorganic salts (e.g., sodium chloride), antimicrobial agents (e.g., benzalkonium chloride), sweeteners, antistatic agents, sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions according to the present disclosure are listed in “Remington: The Science & Practice of Pharmacy”, 19.sup.th ed., Williams & Williams, (1995), and in the “Physician's Desk Reference”, 52.sup.nd ed., Medical Economics, Montvale, N.J. (1998), and in “Handbook of Pharmaceutical Excipients”, Third Ed., Ed. A. H. Kibbe, Pharmaceutical Press, 2000.

Cyclodextrins are a family of oligosaccharides consisting of a macrocyclic ring of glucose subunits joined by α-1,4 glycosidic bonds. Typical cyclodextrins contain a number of glucose monomers ranging from six to eight units in a ring; a (alpha)-cyclodextrin has six glucose subunits, β (beta)-cyclodextrin has seven glucose subunits, and γ (gamma)-cyclodextrin has eight glucose subunits. They are pharmaceutical excipients that may be included in pharmaceutical formulations to improve stability and/or enhance solubility of the pharmaceutically active ingredient. Cyclodextrins have been employed in Remdesivir formulations for this reason. In particular, Gilead's marketed Remdesivir formulation (GS-5734) includes sulfobutylether-β-cyclodextrin (SBECD) to increase the solubility of Remdesivir.

However, the dendrimer-drug conjugates as described herein obviate the need for the addition of cyclodextrin to the formulation. Accordingly, in some embodiments, the composition is free or substantially free of cyclodextrin.

In some embodiments, the composition is free or substantially free of a (alpha)-cyclodextrin. In some embodiments, the composition is free or substantially free of 13 (beta)-cyclodextrin. In some embodiments, the composition is free or substantially free of γ (gamma)-cyclodextrin. In some embodiments, the composition is free or substantially free of sulfobutylether-β-cyclodextrin (SBECD).

In one embodiment, the composition is substantially or entirely free of a solubilisation excipient. By avoiding the use of certain solubilisation excipients, the composition of dendrimer-drug conjugate is less likely to cause side effects. In some embodiments, the composition reduces kidney toxicities compared to Remdesivir formulated with sulfobutylether-β-cyclodextrin (SBECD) (Gilead, GS-5734). In some embodiments, the composition does not cause kidney toxicity. In some embodiments, the composition reduces liver necrosis compared to Remdesivir formulated with sulfobutylether-β-cyclodextrin (SBECD) (Gilead, GS-5734). In some embodiments, the composition does not cause liver necrosis. In some embodiments, the composition reduces obstruction of the renal tubules compared to Remdesivir formulated with sulfobutylether-β-cyclodextrin (SBECD) (Gilead, GS-5734). In some embodiments, the composition does not cause obstruction of the renal tubules. In some embodiments, the composition reduces the need for the patient to receive continuous renal replacement therapy (CRRT).

The dendrimer-drug conjugates of the present disclosure may be formulated in compositions including those suitable for intranasal delivery, oral delivery, pulmonary delivery, inhalation to the lung, by aerosol, or parenteral (including intraperitoneal, ocular, intravenous, subcutaneous, or intramuscular injection) administration. The compositions may conveniently be presented in unit dosage form and may be prepared by any of the methods well known in the art of pharmacy. All methods include the step of bringing the dendrimer-drug conjugate into association with a carrier that constitutes one or more accessory ingredients. Typically, the compositions are prepared by bringing the dendrimer-drug conjugate into association with a liquid carrier to form a solution, or alternatively, bring the dendrimer-drug conjugate into association with formulation components suitable for forming a solid, optionally a particulate product, and then, if warranted, shaping the product into a desired delivery form. Solid formulations of the present disclosure, when particulate, will typically comprise particles with sizes ranging from about 1 nanometer to about 500 microns. In general, for solid formulations intended for intravenous administration, particles will typically range from about 1 nm to about 10 microns in diameter. The composition may contain dendrimer-drug conjugate of the present disclosure that are nanoparticulate having a particulate diameter of below 1000 nm, for example, between 5 and 1000 nm, especially 5 and 500 nm, more especially 5 to 400 nm, such as 5 to 50 nm and especially between 5 and 20 nm. In one example, the composition contains dendrimer-drug conjugates with a mean size of between 5 and 20 nm. In some embodiments, the dendrimer-drug conjugate is polydispersed in the composition, with PDI of between 1.01 and 1.8, especially between 1.01 and 1.5, and more especially between 1.01 and 1.2. In some embodiments, the dendrimer-drug conjugate is polydispersed in the composition with a PDI of about 1.1. In one example, the dendrimer-drug conjugate is monodispersed in the composition.

In some preferred embodiments, the composition is formulated for parenteral delivery. For example, in one embodiment, the formulation may be a sterile, lyophilized composition that is suitable for reconstitution in an aqueous vehicle prior to injection. In one embodiment, the composition is formulated for intravenous injection. In one embodiment, the composition is formulated for intravenous bolus administration. In one embodiment, the composition is formulated for intravenous infusion administration. In one embodiment, the composition is formulated for intramuscular injection. In one embodiment, the composition is formulated for subcutaneous injection. In some embodiments, the composition is a nonaqueous composition for intramuscular injection, for example it may be an oil- and/or organic solvent-based composition.

In one embodiment, a formulation suitable for parenteral administration conveniently comprises a sterile aqueous preparation of the dendrimer-drug conjugate, which may for example be formulated to be isotonic with the blood of the recipient.

In some embodiments, the composition is formulated for intraperitoneal delivery. Any suitable means of delivery may be used. For example, in some embodiments delivery may be by lavage or aerosol. In one embodiment the composition is formulated for intraperitoneal delivery, and is for treatment of viral infections in the peritoneal cavity, which include a Coronavirus (CoV) infection.

In some embodiments, administering the macromolecule to a diseased lung may include delivering the macromolecule to the diseased lung by a pulmonary route. For example, in some embodiments, the macromolecule may be delivered by inhalation, such as inhalation via the mouth and/or nose. In some embodiments, the macromolecule may be delivered by intratracheal instillation or insufflation.

For example, in some embodiments the composition is a solid or an aqueous solution for pulmonary delivery.

For example, in some embodiments, pharmaceutical compositions may be aerosol formulations, nebulized formulations, dry powder or aqueous formulations or insufflation formulations. In some embodiments, pharmaceutical compositions may be included in pressurized metered dose inhalers, dry powder inhalers, nebulizers, soft mist inhalers, and the like. In an embodiment, the composition is suitable for administration in a nasal spray, an oral spray, an inhaler or a nebuliser. For additional discussion, see “Inhaled chemotherapy in lung cancer: future concept of nanomedicine,” International Journal of Nanomedicine, 2012:7, 1551-1572, which is incorporated herein by reference in its entirety.

In some embodiments, the macromolecule is formulated for nasal delivery. In some embodiments, the pharmaceutical formulation may be suitable for intra nasal delivery, such as an aqueous nasal spray formulation or a dry powder nasal spray. Nasal spray formulations may include purified aqueous solutions of the active agent with preservative agents and isotonic agents. Such formulations may be adjusted to a pH and isotonic state compatible with the nasal mucous membranes. In some embodiments, the macromolecule is delivered as a powder, a gel, a liquid, an aerosol or an emulsion. In some embodiments, the pH of the formulation is about 4.5 to about 7.42. In some embodiments, the pH of the formulation is about 5 to about 7. In some embodiments, the pH of the formulation is about 5 to about 6.5. In some embodiments, the pH is about 5.5 to about 6.5. In other embodiments, the pH is about 7.4.

In some embodiments, the osmolality of the formulation is about 200 to about 700 Osmol/kg. In some embodiments, the osmolality of the formulation is about 300 to about 600 Osmol/kg. In some embodiments, the osmolality of the formulation is about 300 to about 700 Osmol/kg In some embodiments, the osmolality of the formulation is about 200 to about 400 Osmol/kg, more preferably about 280 Osmol/Kg. Osmolality regulators include NaCl, lysine, CaCl₂, sodium citrate and pH regulators include H₂SO₄, NaOH, tromethamine, HCl.

In some embodiments, the macromolecule is formulated for delivery to the lung. Neutral pH and tonicity are important factors for lower respiratory delivery to avoid bronchoconstriction in patients with respiratory impairment, as the lungs are poorly buffered.

In some embodiments, the pharmaceutical formulation may be a dry powder with particle sizes greater than 0.5 μm and less than 50 μm. In some embodiments, the particle size is less than 5 um, greater than 1 um.

In some embodiments, the macromolecule may have a particulate size of less than about 100 nm. In other embodiments, macromolecule may have a particulate size between about 1 and about 10 nm, between about 2 and about 8 nm, and between about 3 and about 6 nm by DLS. In some embodiments, the macromolecules may have a mean size of about 5 nm by DLS (at 1 mg/ml in 10-2M NaCl). In some embodiments, the macromolecule may have a molecular weight of less than 30 kDa, between about 10 to about 30 kDa, and between about 10 to about 20 kDa.

Examples of ingredients suitable for nasal or oral delivery include are provided in the Table below.

| | IIG for nasal | |
|---|---|---|
| Ingredients | route, % w/w | Function |
| Alcohol (ethanol), 200 proof | 2 | Co-solvent |
| Anhydrous dextrose | 0.5 | tonicity |
| Anhydrous trisodiumcitrate | 0.0006 | buffer |
| Benzyl alcohol | 0.0366 | preservative |
| Benzalkonium chloride | 0.119 | preservative |
| Butylated hydroxyanisole | 0.0002 | antioxidant |
| Cellulose microcrystalline | 2 | Suspending agent, |
| | | stabilizer |
| Chlorobutanol | 0.5 | preservative |
| Carboxymethyl cellulose Na | 0.15 | Suspending agent |
| Hydroxypropyl | (4- topical) | |
| methylcellulose | | |
| Edetate disodium | 0.5 | Chelator, |
| | | antioxidant |
| Hydrochloric acid | Not reported | pH adjustment |
| Methylparaben | 0.7 | preservative |
| Oleic acid | 0.132 | Penetration |
| | | enhancer |
| PEG400 | 20 | Surfactant, co- |
| | | solvent |
| PEG3500 | 1.5 | surfactant |
| Phenylethyl alcohol | 0.254 | Preservative, |
| | | masking agent |
| Polyoxyl 400 stearate | 15 | surfactant |
| Polysorbate 20 | 2.5 | surfactant |
| Polysorbate 80 | 10 | surfactant |
| Propylene glycol | 20 | Co-solvent |
| Propylparaben | 0.3 | Preservative |
| Sodium chloride | 1.9 | tonicity |
| Sodium hydroxide | 0.004 | pH adjustment |
| Sulfuric acid | 0.4 | pH adjustment |
| Succinic Acid | | |
| Disodium Succinate | | |
| Zinc Acetate | | |
| Sugars, or flavouring agents | | |
| e.g. Sodium Saccharin | | |

The rapid mucocillary clearance in the nasal cavity and presence of nasal lysozymes and macrophage can present challenges to mucosal delivery. Mucoadhesive excipients may be required. Depending on the intended mode of administration, the compositions may comprise a bioadhesive agent. In an embodiment, the bioadhesive is a mucoadhesive agent mucoadhesive polymer. A mucoadhesive or bioadhesive agent may alter the viscosity, rheology and/or the ciliary beating frequency (CBF). Examples of mucoadhesive polymers include poly(acrylates), chitosan, cellulose and derivatives including carboxymethylcellulose and hydroxypropyl cellulose, hyaluronic acid derivatives, pectin, traganth, starch, poly(ethylene glycol), sulfated polysaccharides, carrageenan, sodium alginate, polyvinyl alcohol, polyvinylpyrrolidone, acacia gum, alginic acid, and gelatine. In an embodiment, the composition may comprise a nasal mucoadhesive component. However, viscosity should not impede airflow. In some embodiments, viscosity of the formulation is between 1 and 10000 cP, or between 1 and 1000 cP, or between 100 and 1000 cP, or between 100 and 500 cP, or between 100 and 400 cP, or between 150 and 300 cP, or between 150 and 250 cP. In some embodiments, the kinematic viscosity of the solution is below 1000, or below 500 mm2·s-1. For lung delivery, viscosity should be low. In some embodiments, the viscosity is less than 200 cP. In some embodiments, the viscosity is less than 100 cP.

In some embodiments, the pharmaceutical composition may also include any other therapeutic ingredients, surfactants, propellants, stabilizers, or the like. The carrier(s) must be pharmaceutically acceptable in the sense of being compatible with the other ingredients of the formulation and not unduly deleterious to the recipient thereof.

In some embodiments, the pharmaceutical formulation may be a dry powder with particle sizes greater than 1 0.5 μm and smaller less than 50 μm. In some embodiments, the particle size is less than 5 μm, greater than 1 uμm greater than 10 μm.

In some embodiments, a particle diameter of 1 to about 5 μm is good for delivery to the lower airway; from 5 to 10 μm particles deposit mostly in the trachea and bronchi, while diameter >10 μm particles deposit mostly in the nose. Usually particles less than 10 μm median aerodynamic diameter, can reach the lower airways during nasal breathing. In one embodiment, the mean particle size is from about 0.21 to about −200 μm. In one embodiment, the mean particle size is from about 1 to about 200 μm. In one embodiment, the mean particle size is from about 1 to about 50 μm. In one embodiment, the mean particle size is from about 1 to about 20 μm. In one embodiment, the mean particle size is from about 1 to about 5 μm. The composition may be a liquid, gel or powder. In some embodiments, suitable for lower airway delivery the Dv90 is about 5 to 20 μm. In some embodiments, suitable for lower airway delivery the Dv50 is about 5 to 10 μm. In some embodiments, suitable for lower airway delivery the Dv10 is about 1 to 5 μm. In some embodiments, suitable for nasal delivery the Dv10 is greater than about 10, 15 or 20 μm. In some embodiments, suitable for nasal delivery the Dv50 is greater than about 20, 40 or 60 μm. In some embodiments, suitable for nasal delivery the Dv90 is greater than about 60, 80 or 1000 μm. The dendrimer-drug conjugates may also conveniently be provided in the form of a solid (e.g. powder) composition for reconstitution, e.g. for admixing with an aqueous diluent such as saline prior to administration, e.g. by injection or infusion. Such a composition may for example contain the conjugate and suitable excipients if required, for example a buffer or preservative. Accordingly, in some embodiments, the pharmaceutical composition comprising the dendrimer-drug conjugate is provided in the form of a solid composition for reconstitution. In some embodiments, a kit comprising a drug-dendrimer conjugate composition and instructions for reconstitution.

As discussed above, the dendrimer-drug conjugates of the present disclosure may for example be administered in combination with one or more additional pharmaceutically active agents. In some embodiments, the dendrimer-drug conjugate is provided in combination with a further active. In some embodiments, a composition is provided which comprises a dendrimer-drug conjugate as defined herein or a pharmaceutically acceptable salt thereof, one or more pharmaceutically acceptable carriers, and one or more additional pharmaceutically active agents, e.g., a further therapeutic agent used for therapy of a viral infection, such as a SPL7013 (astodrimer or a salt thereof, such as a sodium salt), or a further therapeutic agent used for therapy of a bacterial infection.

As discussed above, Remdesivir has low water solubility, requires formulating in special excipients, and requires lengthy administration times over a prolonged period of time (e.g. over 5-10 days consecutively).

The dendrimer-drug conjugate typically provides increased solubility of Remdesivir nucleoside, in comparison to free Remdesivir (i.e., Remdesivir unbound to the dendrimer-drug conjugate). In some embodiments, the conjugate provides increased aqueous solubility. In some embodiments, the conjugate provides increased non-aqueous solubility, e.g. in an organic solvent or mixture of organic solvents.

Aqueous solubility may for example be determined by dissolving an amount of the sample in water at room temperature, e.g. at 25° C.

Thus, in some embodiments, the composition is aqueous and comprises dendrimer-drug conjugate in solution, and wherein the composition comprises a greater concentration in moles of Remdesivir nucleoside than the maximal concentration of Remdesivir in water at 25° C.

In some embodiments, the dendrimer-drug conjugate provides increased aqueous solubility of Remdesivir nucleoside of at least 1.5 times, at least 2 times, at least 3 times, at least 5 times, at least 10 times, at least 20 times, at least 30 times, at least 40 times, or at least 50 times the aqueous solubility of free Remdesivir, when measured at 25° C.

In some embodiments, the aqueous solubility of the dendrimer-drug conjugate is at least 50 mg/mL, at least 75 mg/mL, at least 100 mg/mL, at least 150 mg/mL, at least 200 mg/mL, at least 300 mg/mL, when measured at 25° C.

Dendrimer-drug conjugates according to the present disclosure have been found to demonstrate low viscosity and good solubility, such that they have good properties for administration to subjects, for example by subcutaneous administration. In some embodiments, the dendrimer-drug conjugates are provided in the form of a composition (e.g. an injectable composition, for example for subcutaneous injection) comprising a concentration of dendrimer in the range of from 100 mg/ml to 400 mg/ml, for example about 100 mg/ml, about 150 mg/ml, about 200 mg/ml, about 250 mg/ml, about 300 mg/ml, about 350 mg/ml or about 400 mg/ml.

In some embodiments, the effective dose of dendrimer-drug conjugate or composition is delivered in a volume of less than or equal to 5 ml, 4 ml, 3 ml, 2.5 ml, or 2 ml. In some embodiments, dendrimer-drug conjugate or composition is delivered in a single injection, or alternatively, as two or more injections in temporal proximity.

### Drug-Dendrimer Conjugate Synthesis

The dendrimers of the present disclosure may be prepared by any suitable method, for example by reacting a drug moiety-containing precursor with a dendrimeric intermediate already containing a hydrophilic polymeric group to introduce the pharmaceutically active agent, by reacting a hydrophilic polymeric group-containing precursor with a dendrimeric intermediate already containing a drug moiety, or by reacting an intermediate comprising the residue of a lysine group, a drug moiety and a hydrophilic polymeric group with a dendrimeric intermediate. Protection and deprotection steps using protecting groups may be utilised as desired.

In some embodiments where the drug moiety is:

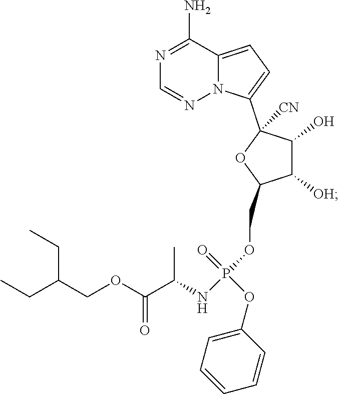

there is provided a process for producing a dendrimer-drug conjugate as defined herein, comprising:
a) reacting a nucleoside analogue intermediate which is:

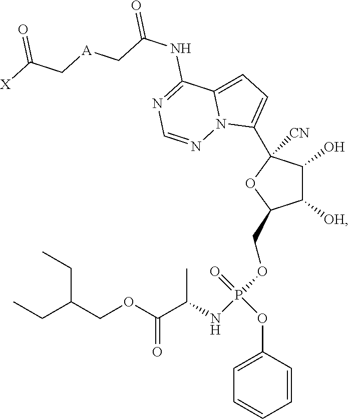

wherein A is O, S, or NMe, X is —OH or a leaving group, or wherein X together with the C(O) group to which it is attached forms a carboxylate salt;

with a dendrimeric intermediate which comprises:
i) a core unit (C); and
ii) building units (BU), each building unit being a lysine residue or an analogue thereof;
wherein the core unit is covalently attached to two building units via amide linkages, each amide linkage being formed between a nitrogen atom present in the core unit and the carbon atom of an acyl group present in a building unit;
wherein building units of different generations are covalently attached to one another via amide linkages formed between a nitrogen atom present in one building unit and the carbon atom of an acyl group present in another building unit;
   the dendrimer further comprising:
a plurality of second terminal groups (T2) each comprising a hydrophilic polymeric group;
or a salt thereof;
under amide coupling conditions;
or
b) reacting a hydrophilic polymeric intermediate which is:

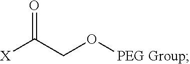

wherein PEG Group is a PEG-containing group, and
X is —OH or a leaving group, or wherein X together with the C(O) group to which it is attached forms a carboxylate salt;
with a dendrimeric intermediate which comprises:
i) a core unit (C); and
ii) building units (BU), each building unit being a lysine residue or an analogue thereof;
wherein the core unit is covalently attached to two building units via amide linkages, each amide linkage being formed between a nitrogen atom present in the core unit and the carbon atom of an acyl group present in a building unit;
wherein building units of different generations are covalently attached to one another via amide linkages formed between a nitrogen atom present in one building unit and the carbon atom of an acyl group present in another building unit;
   the dendrimer further comprising:
a plurality of first terminal groups (T1) each comprising a drug moiety comprising a Remdesivir nucleoside, the drug moiety being covalently attached to a linker group of formula

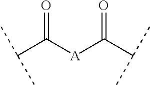

wherein A is —CH₂OCH₂—, —CH₂SCH₂— or —CH₂N(Me)CH₂—;
or a salt thereof;
under amide coupling conditions;
or
c) reacting a surface unit intermediate which is:

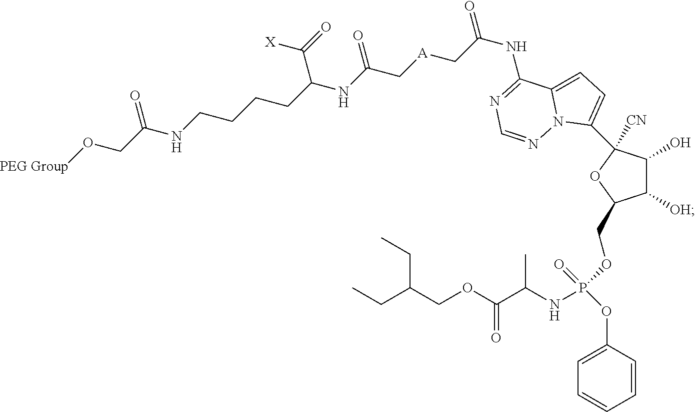

wherein PEG Group is a PEG-containing group, A is O, S or NMe, and
X is —OH or a leaving group, or wherein X together with the C(O) group to which it is attached forms a carboxylate salt;
with a dendrimeric intermediate comprising:
i) a core unit (C); and
ii) building units (BU), each building unit being a lysine residue or an analogue thereof;
wherein the core unit is covalently attached to two building units via amide linkages, each amide linkage being formed between a nitrogen atom present in the core unit and the carbon atom of an acyl group present in a building unit;
wherein building units of different generations are covalently attached to one another via amide linkages formed between a nitrogen atom present in one building unit and the carbon atom of an acyl group present in another building unit;
and wherein nitrogen atoms present in the outer building units of the dendrimeric intermediate are unsubstituted;
or a salt thereof;
under amide coupling conditions.

Process variants a), b) and c) involve formation of amide bonds by reaction of —C(O)X groups with amine groups present in the dendrimeric intermediates. Any suitable amide formation conditions may be used. Examples of typical conditions include the use of a suitable solvent (for example dimethylformamide) optionally a suitable base, and at a suitable temperature (for example ambient temperature, e.g. in the range of from 15 to 30° C.). Where X is a leaving group, any suitable leaving group may be used, for example an activated ester. Where X is an —OH group or where X together with the C(O) group to which it is attached forms a carboxylate salt, the group will typically be converted to a suitable leaving group prior to reaction with a dendrimeric intermediate, for example by use of a suitable amide coupling reagent such as PyBOP.

Any suitable isolation and/or purification technique may be utilised, for example the dendrimer may be obtained by dissolution in a suitable solvent and precipitation by addition into an antisolvent.

The drug moiety intermediates used in variant a) may themselves be obtained, for example, by reaction of the drug moiety comprising Remdesivir nucleoside (optionally appropriately protected) with a suitable anhydride (e.g. diglycolic anhydride or thiodiglycolic anhydride), for example in the presence of a suitable solvent and a suitable base, and optionally with the use of suitable protecting group chemistry to protect, for example, alcohol functionality.

The surface unit intermediate used in variants c) may itself be obtained, for example, by:
i) reacting a PEG intermediate which is:

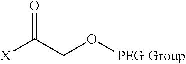

wherein PEG Group is a PEG-containing group, and
X is —OH or a leaving group, or wherein X together with the C(O) group to which it is attached forms a carboxylate salt;
with

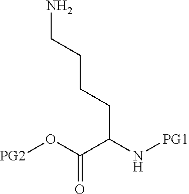

wherein PG1 is an amine protecting group (such as a Boc, Cbz or, preferably, Fmoc group), and PG2 is an acid protecting group (such as a methyl or benzyl ester);
ii) deprotecting PG1;
iii) reacting the product of step ii) with a nucleoside analogue intermediate which is:

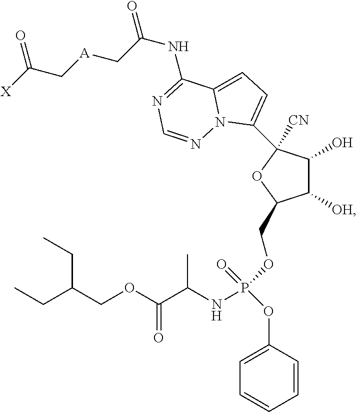

wherein A is O, NMe or S, X is —OH or a leaving group, or wherein X together with the C(O) group to which it is attached forms a carboxylate salt; and
iv) deprotecting PG2.

The dendrimeric intermediate used in variant a) may itself be obtained by, for example, a sequential process involving:
i) reaction of a core unit (C) containing amino groups, with building units which are protected lysines or analogues thereof, which contain a —C(O)X group, wherein X is —OH or a leaving group or —CO(X) forms a carboxylate salt, and in which the amino groups present in the lysines or analogues thereof are protected, to form amide linkages between the core unit and building units;
ii) deprotecting protecting groups present on the building units;
iii) reacting free amino groups present on the building units with further building units which are protected lysines or analogues thereof, which contain a —C(O)X group, wherein X is —OH or a leaving group or —CO(X) forms a carboxylate salt, and in which the amino groups present in the lysines or analogues thereof are protected, to form amide linkages between the different generations of building units;
iv) deprotecting protecting groups present on the building units;
v) repeating steps iii) and iv) until a four generation building unit is produced;
vi) reacting free amino groups present on the building units with

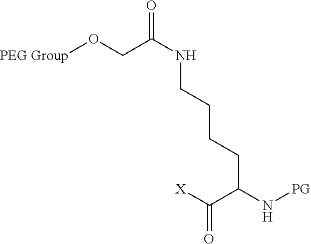

wherein PG is a protecting group, and wherein X is —OH or a leaving group, or wherein X together with the C(O) group to which it is attached forms a carboxylate salt, to form amide linkages therebetween; and
vii) deprotecting the protecting groups PG.
Alternatively, the dendrimeric intermediate used in variant a) may be obtained, for example, by carrying out steps i) to v) as described above, and:
vi) reacting free amino groups present on the building units with further building units which are protected lysines or analogues thereof, which contain a —C(O)X group, wherein X is —OH or a leaving group or —CO(X) forms a carboxylate salt, and in which the amino groups present in the lysines or analogues thereof are orthogonally protected, to form amide linkages between the different generations of building units;
vii) deprotecting a first set of amino protecting groups;
viii) reacting free amino groups present on the building units with

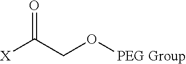

wherein PEG Group is a PEG-containing group, and X is —OH or a leaving group, or wherein X together with the C(O) group to which it is attached forms a carboxylate salt;
vii) deprotecting a second set of amino protecting groups.

The dendrimeric intermediate used in variant b) may itself be obtained, for example, by carrying out steps i) to v) as described above in relation to variant a), and:
vi) reacting free amino groups present on the building units with

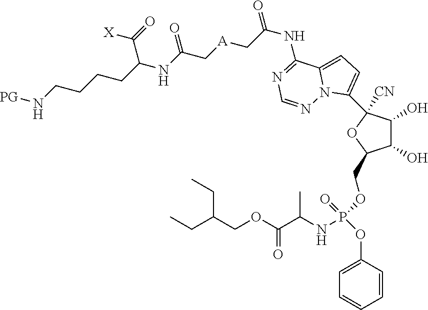

wherein A is O or S, PG is a protecting group, and wherein X is —OH or a leaving group, or wherein X together with the C(O) group to which it is attached forms a carboxylate salt, to form amide linkages therebetween; and
vii) deprotecting the protecting groups PG.
Alternatively, the dendrimeric intermediate used in variant b) may be obtained, for example, by carrying out steps i) to v) as described above, and:
vi) reacting free amino groups present on the building units with further building units which are protected lysines or analogues thereof, which contain a —C(O)X group, wherein X is —OH or a leaving group or —CO(X) forms a carboxylate salt, and in which the amino groups present in the lysines or analogues thereof are orthogonally protected, to form amide linkages between the different generations of building units;
vii) deprotecting a first set of amino protecting groups;
viii) reacting free amino groups present on the building units with

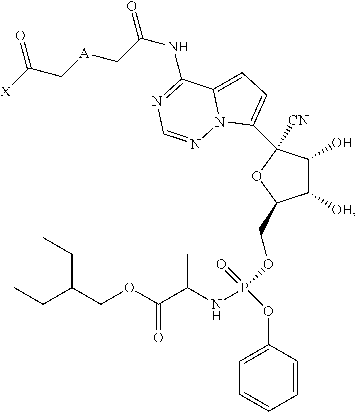

wherein A is O, NMe or S, X is —OH or a leaving group, or wherein X together with the C(O) group to which it is attached forms a carboxylate salt;
vii) deprotecting a second set of amino protecting groups.

The dendrimeric intermediate used in variant c) may itself be obtained, for example, by carrying out steps i) to v) as described above in relation to variant a).

Dendrimers containing a core (e.g. a BHA-Lys core) and lysine or lysine analogue building units may for example be synthesised as described in WO02/079299 and WO2012/167309.

The present disclosure also provides synthetic intermediates useful in producing the dendrimers. Accordingly, there is also provided an intermediate for producing a dendrimer which is

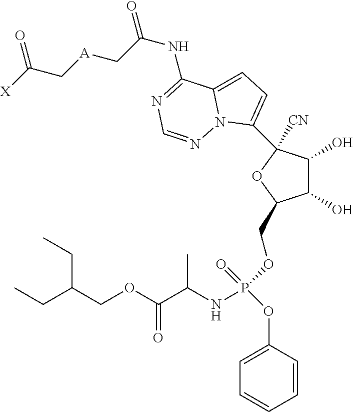

wherein A is O, NMe or S, X is —OH or a leaving group, or wherein X together with the C(O) group to which it is attached forms a carboxylate salt. Such an intermediate may be produced, for example, as described above.

There is also provided an intermediate for producing a dendrimer which is

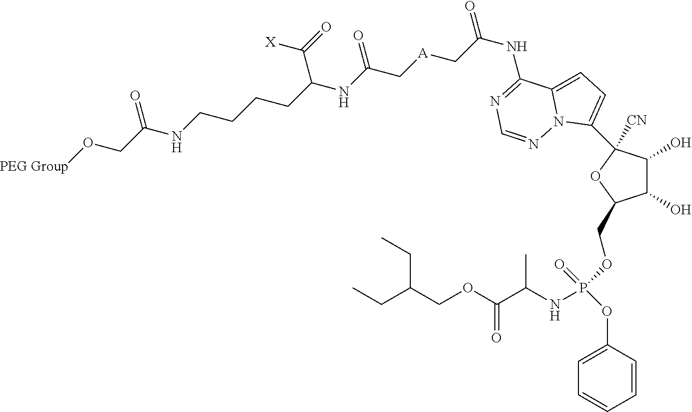

wherein PEG Group is a PEG-containing group, A is O or S, and
X is —OH or a leaving group, or wherein X together with the C(O) group to which it is attached forms a carboxylate salt. Such an intermediate may be produced, for example, as described above.

The present disclosure will now be described with reference to the following examples which illustrate some particular aspects of the present disclosure. However, it is to be understood that the particularity of the following description of the present disclosure is not to supersede the generality of the preceding description of the present disclosure.

### EXAMPLES

### Example 1: Dendrimer-Drug Conjugate Synthesis

TABLE 1
Table of Compounds Synthesised

| Compound | | |
|---|---|---|
| No. | Compound Description | Structure |
| RHa-1 | Remdesivir (or RDV) | 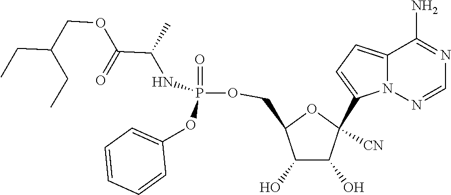 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1] |
| RHa-2 | 3′O-Glu-Remdesivir | 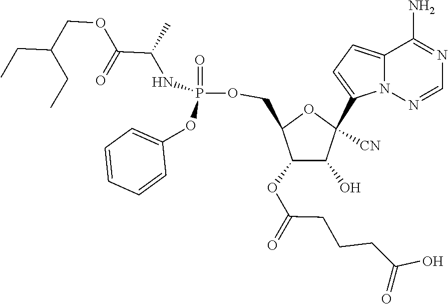 [SMILES: CCC(CC)COC(=O)[C@H](C)N[P@](=O)(OC[C@H]1O[C@@](C#N)(c2ccc3c(N)ncnn23)[C@H](O)[C@@H]1OC(=O)CCCC(=O)O)Oc1ccccc1] |
| RHa-3 | BHALys[Lys]₂[Lys]₄[Lys]₈[Lys]₁₆ | |
| | [Lys]₃₂[(α-NH-3′O-Glu-Remdesivir)₃₂ | |
| | (ε-NH-COPEG₂₀₀₀)₃₂] | |
| RHa-4 | 6N-TDA-Remdesivir | 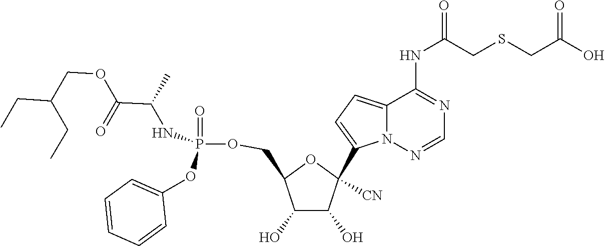 [SMILES: CCC(CC)COC(=O)[C@H](C)N[P@](=O)(OC[C@H]1O[C@@](C#N)(c2ccc3c(NC(=O)CSCC(=O)O)ncnn23)[C@H](O)[C@@H]1O)Oc1ccccc1] |
| RHa-5 | BHALys[Lys]₂[Lys]₄[Lys]₈[Lys]₁₆ | |
| | [Lys]₃₂[(α-NH-6N-TDA-Remdesivir)₃₂ | |
| | (ε-NH-COPEG₂₀₀₀)₃₂] | |
| RHa-6 | BHALys[Lys]₂[Lys]₄[Lys]₈[Lys]₁₆ | |
| | [Lys]₃₂[(α-NH₂)₃₂(ε-NH- | |
| | COPEG₂₀₀₀)₃₂]•32TFA | |
| RHa-7 | 6N-DMF-Remdesivir | 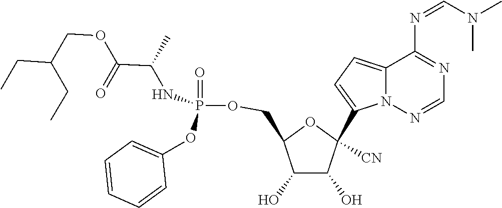 [SMILES: CCC(CC)COC(=O)[C@H](C)N[P@](=O)(OC[C@H]1O[C@@](C#N)(c2ccc3c(/N=C\N(C)C)ncnn23)[C@H](O)[C@@H]1O)Oc1ccccc1] |
| RHa-8 | 6N-DMF-3′O-DGA-Remdesivir | 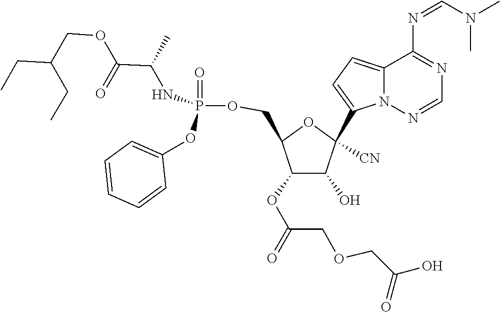 [SMILES: CCC(CC)COC(=O)[C@H](C)N[P@](=O)(OC[C@H]1O[C@@](C#N)(c2ccc3c(/N=C\N(C)C)ncnn23)[C@H](O)[C@@H]1OC(=O)COCC(=O)O)Oc1ccccc1] |
| RHa-9 | 6N-DMF-2′O,3′O-bis-DGA-Remdesivir | 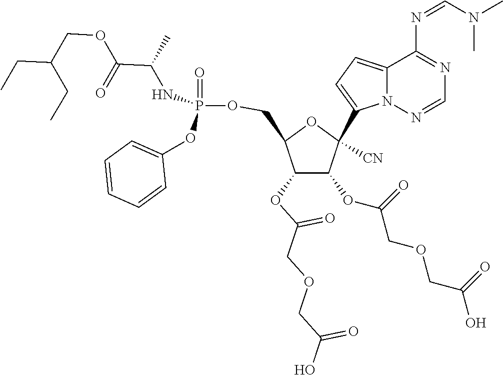 [SMILES: CCC(CC)COC(=O)[C@H](C)N[P@](=O)(OC[C@H]1O[C@@](C#N)(c2ccc3c(/N=C\N(C)C)ncnn23)[C@H](OC(=O)COCC(=O)O)[C@@H]1OC(=O)COCC(=O)O)Oc1ccccc1] |
| RHa-10 | 3′O-DGA-Remdesivir | 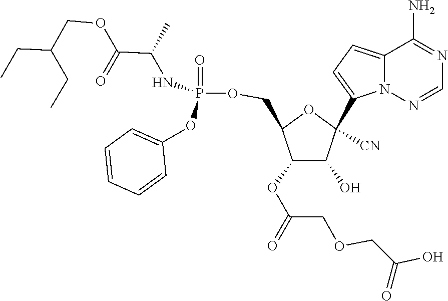 [SMILES: CCC(CC)COC(=O)[C@H](C)N[P@](=O)(OC[C@H]1O[C@@](C#N)(c2ccc3c(N)ncnn23)[C@H](O)[C@@H]1OC(=O)COCC(=O)O)Oc1ccccc1] |
| RHa-11 | BHALys[Lys]₂[Lys]₄[Lys]₈[Lys]₁₆ | |
| | [Lys]₃₂[(α-NH-3′O-DGA-Remdesivir)₃₂ | |
| | (ε-NH-COPEG₂₀₀₀)₃₂] | |
| RHa-12 | 6N-DMF-3′O-TDA-Remdesivir | 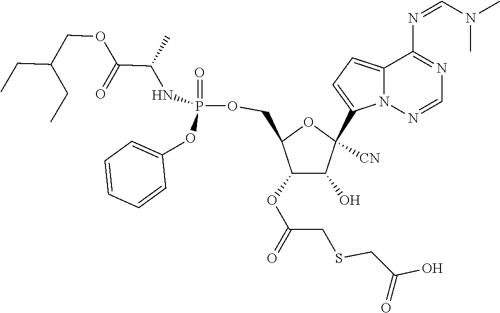 [SMILES: CCC(CC)COC(=O)[C@H](C)N[P@](=O)(OC[C@H]1O[C@@](C#N)(c2ccc3c(/N=C\N(C)C)ncnn23)[C@H](O)[C@@H]1OC(=O)CSCC(=O)O)Oc1ccccc1] |
| RHa-13 | 6N-DMF-2′O,3′O-bis-TDA-Remdesivir | 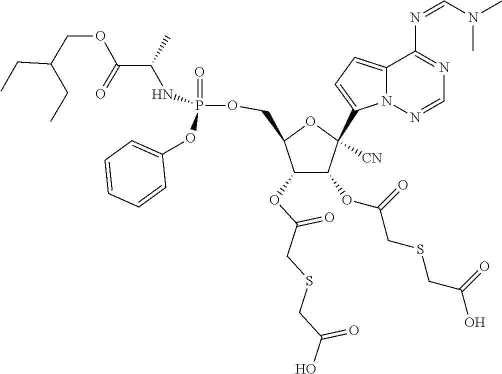 [SMILES: CCC(CC)COC(=O)[C@H](C)N[P@](=O)(OC[C@H]1O[C@@](C#N)(c2ccc3c(/N=C\N(C)C)ncnn23)[C@H](OC(=O)CSCC(=O)O)[C@@H]1OC(=O)CSCC(=O)O)Oc1ccccc1] |
| RHa-14 | 3′O-TDA-Remdesivir | 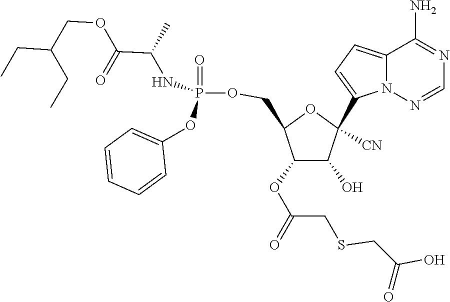 [SMILES: CCC(CC)COC(=O)[C@H](C)N[P@](=O)(OC[C@H]1O[C@@](C#N)(c2ccc3c(N)ncnn23)[C@H](O)[C@@H]1OC(=O)CSCC(=O)O)Oc1ccccc1] |
| RHa-15 | BHALys[Lys]₂[Lys]₄[Lys]₈[Lys]₁₆ | |
| | [Lys]₃₂[(α-NH-3′O-TDA-Remdesivir)₃₂ | |
| | (ε-NH-COPEG₂₀₀₀)₃₂] | |
| RHa-16 | BHALys[Lys]₂[Lys]₄[Lys]₈[Lys]₁₆ | |
| | [Lys]₃₂[(α-NH₂)₃₂(ε-NH- | |
| | COPEG₁₀₀₀)₃₂]•32 TFA | |
| RHa-17 | BHALys[Lys]₂[Lys]₄[Lys]₈[Lys]₁₆ | |
| | [Lys]₃₂[(α-NH-6N-TDA-Remdesivir)₃₂ | |
| | (ε-NH-COPEG₁₀₀₀)₃₂] | |
| RL-1 | GS-441524 | 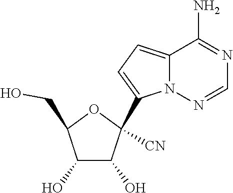 [SMILES: N#C[C@@]1(c2ccc3c(N)ncnn23)O[C@H](CO)[C@@H](O)[C@H]1O] |
| RL-2 | 2′O-,3′O-Acetonide-GS-441524 | 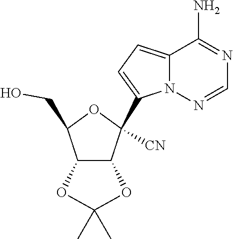 [SMILES: CC1(C)O[C@H]2[C@@H](O1)[C@](C#N)(c1ccc3c(N)ncnn13)O[C@@H]2CO] |
| RL-3 | 2′O-,3′O-Acetonide-6N-formamidine-GS- 441524 | 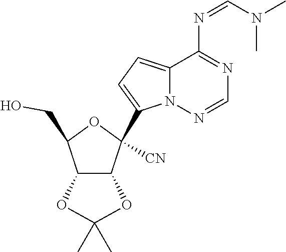 [SMILES: CN(C)/C=N\c1ncnn2c([C@]3(C#N)O[C@H](CO)[C@H]4OC(C)(C)O[C@H]43)ccc12] |
| RL-4 | 5′O-DGA-GS-441524 | 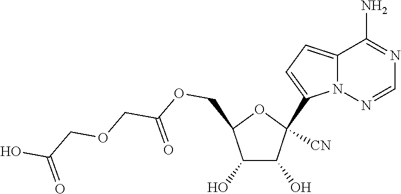 [SMILES: N#C[C@@]1(c2ccc3c(N)ncnn23)O[C@H](COC(=O)COCC(=O)O)[C@@H](O)[C@H]1O] |
| RL-5 | 2′O-,3′O-acetonide-5′O-TDA-GS-441524 | 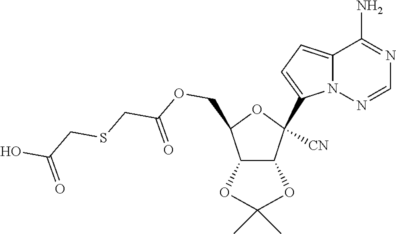 [SMILES: CC1(C)O[C@H]2[C@@H](O1)[C@](C#N)(c1ccc3c(N)ncnn13)O[C@@H]2COC(=O)CSCC(=O)O] |
| RL-6 | 5′O-TDA-GS-441524 | 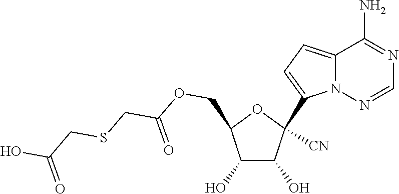 [SMILES: N#C[C@@]1(c2ccc3c(N)ncnn23)O[C@H](COC(=O)CSCC(=O)O)[C@@H](O)[C@H]1O] |
| RL-7 | BHALys[Lys]₂[Lys]₄[Lys]₈[Lys]₁₆ [Lys]₃₂[(α-NH-5′O-DGA-GS-441524)₃₂ (ε-NH-COPEG₂₀₀₀)₃₂] | 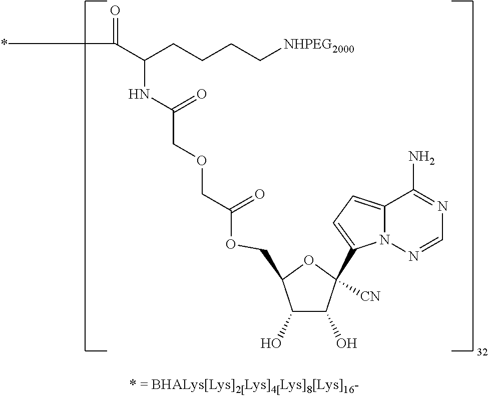 |
| RL-8 | BHALys[Lys]₂[Lys]₄[Lys]₈[Lys]₁₆ [Lys]₃₂[(α-NH-5′O-TDA-GS-441524)₃₂ (ε-NH-COPEG₂₀₀₀)₃₂] | 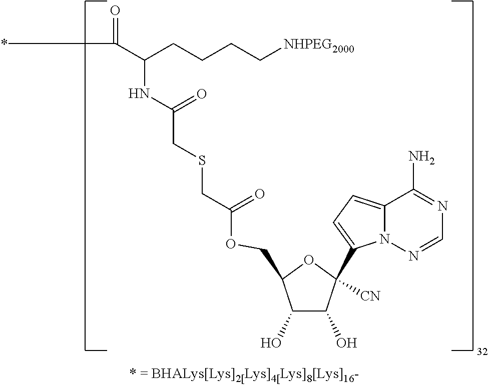 |

TABLE 2
Definitions

| Abbreviation | Structure |
|---|---|
| —NHCOPEG₁₀₀₀ | 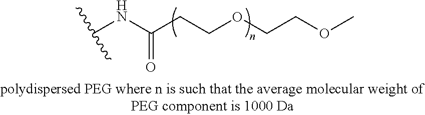 |
| —NHCOPEG₂₀₀₀ | 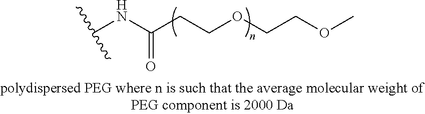 |
| —3′O-Glu-Remdesivir | 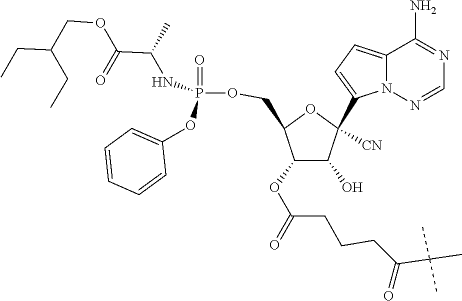 |
| —6N-TDA-Remdesivir | 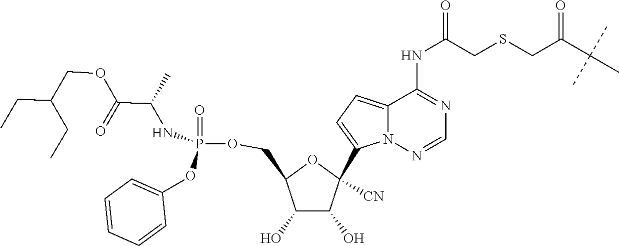 |
| 6N-DMF-Remdesivir | 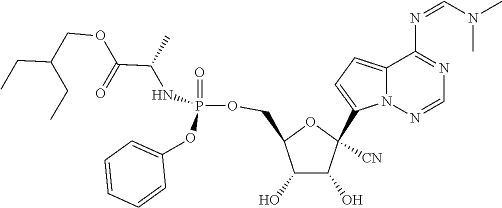 [SMILES: CCC(CC)COC(=O)[C@H](C)N[P@](=O)(OC[C@H]1O[C@@](C#N)(c2ccc3c(/N=C\N(C)C)ncnn23)[C@H](O)[C@@H]1O)Oc1ccccc1] |
| 6N-DMF-3′O-DGA- Remdesivir | 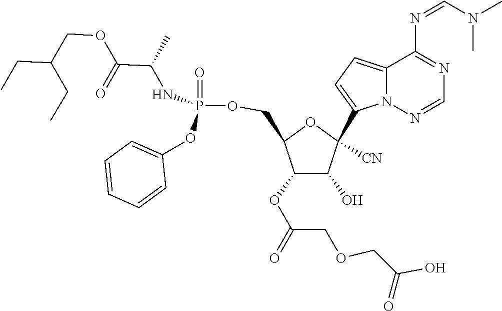 [SMILES: CCC(CC)COC(=O)[C@H](C)N[P@](=O)(OC[C@H]1O[C@@](C#N)(c2ccc3c(/N=C\N(C)C)ncnn23)[C@H](O)[C@@H]1OC(=O)COCC(=O)O)Oc1ccccc1] |
| 6N-DMF-2′O,3′O-bis- DGA-Remdesivir | 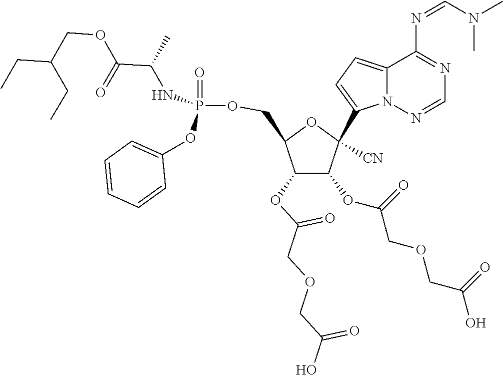 [SMILES: CCC(CC)COC(=O)[C@H](C)N[P@](=O)(OC[C@H]1O[C@@](C#N)(c2ccc3c(/N=C\N(C)C)ncnn23)[C@H](OC(=O)COCC(=O)O)[C@@H]1OC(=O)COCC(=O)O)Oc1ccccc1] |
| 3′O-DGA-Remdesivir | 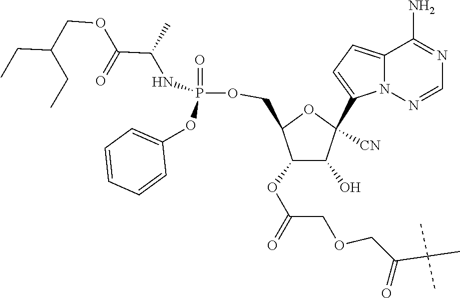 |
| 6N-DMF-3′O-TDA- Remdesivir | 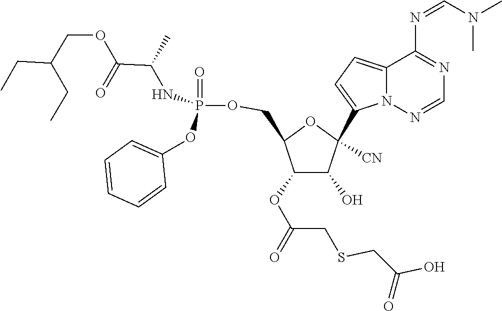 [SMILES: CCC(CC)COC(=O)[C@H](C)N[P@](=O)(OC[C@H]1O[C@@](C#N)(c2ccc3c(/N=C\N(C)C)ncnn23)[C@H](O)[C@@H]1OC(=O)CSCC(=O)O)Oc1ccccc1] |
| 6N-DMF-2′O,3′O-bis- TDA-Remdesivir | 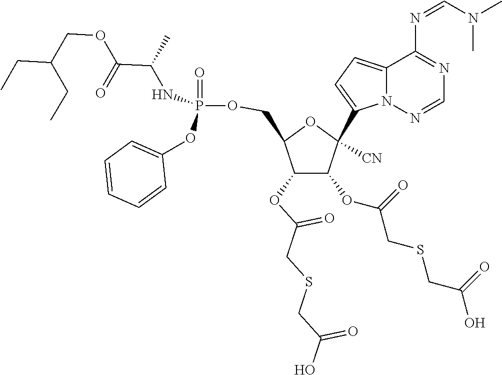 [SMILES: CCC(CC)COC(=O)[C@H](C)N[P@](=O)(OC[C@H]1O[C@@](C#N)(c2ccc3c(/N=C\N(C)C)ncnn23)[C@H](OC(=O)CSCC(=O)O)[C@@H]1OC(=O)CSCC(=O)O)Oc1ccccc1] |
| 3′O-TDA-Remdesivir | 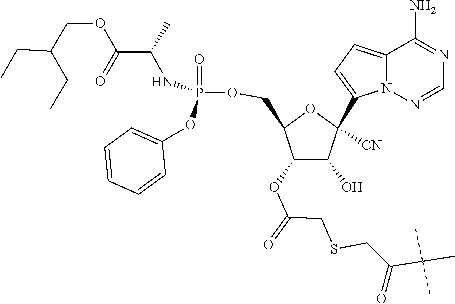 |
| —5′O-DGA-GS-441524 | 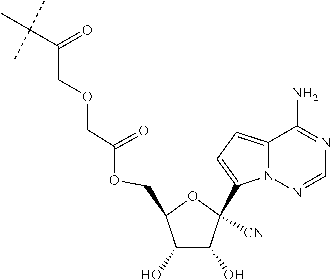 |
| —5′O-TDA-GS-441524 | 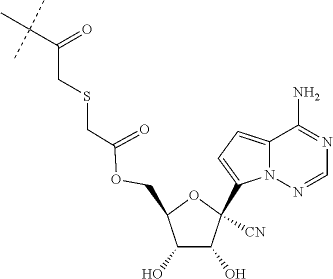 |
| 2′O, 3′O-Acetonide- 6N-formamidine-GS- 441524 | 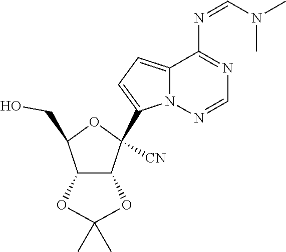 [SMILES: CN(C)/C=N\c1ncnn2c([C@]3(C#N)O[C@H](CO)[C@H]4OC(C)(C)O[C@H]43)ccc12] |
| 2′O, 3′O-Acetonide- GS-441524 | 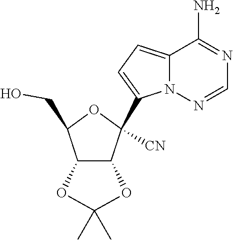 [SMILES: CC1(C)O[C@H]2[C@@H](O1)[C@](C#N)(c1ccc3c(N)ncnn13)O[C@@H]2CO] |
| GS-441524 | 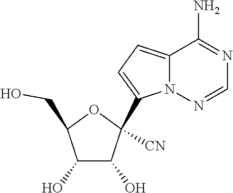 [SMILES: N#C[C@@]1(c2ccc3c(N)ncnn23)O[C@H](CO)[C@@H](O)[C@H]1O] |

TABLE 3
Abbreviations

| Abbreviation | Name |
|---|---|
| aq | Aqueous |
| CP | Centipoise |
| BHA | Benzhydryl alcohol |
| CD₃CN | Deuterated acetonitrile |
| CD₃OD | Deuterated methanol |
| DCM | Dichloromethane |
| DIPEA | N,N-Diisopropylethylamine |
| DMF | N,N-Dimethylformamide |
| DMSO | Dimethylsulfoxide |
| Equiv | Equivalent |
| ESI MS | Electrospray mass spectrometry |
| EtOH | Ethanol |
| H | Hour |
| LCMS | Liquid chromatography mass spectrometry |
| Lys | Lysine |
| MeCN | Acetonitrile |
| MeOH | Methanol |
| mL | Millilitre |
| Mmol | Millimole |
| μL | Microlitre |
| NMM | N-Methylmorpholine |
| NMR | Nuclear Magnetic Resonance |
| PEG | Polyethylene glycol |
| PyBOP | Benzotriazol-1-yl-oxytri-pyrrolidinophosphonium |
| | hexafluorophosphate |
| rt | room temperature |
| Rₜ | Retention time |
| SEC | Size exclusion chromatography |
| TEA | Triethylamine |
| TFA | Trifluoroacetic acid |
| TFF | Tangential Flow Filtration |

As denoted in the Examples, “RDV” refers to Remdesivir in a form wherein Remdesivir is not conjugated to a dendrimer or other macromolecule. That is, RDV refers to Remdesivir in its “free” or “unconjugated” form.

### General Procedures for Analysis

### LCMS

LCMS Method 1: LCMS was recorded on Phenomenex Kinetex® 2.6 μm 2.1×75 mm C18 column using a ternary solvent system consisting of solvent A (water), solvent B (acetonitrile) and solvent C (10% of 1% v/v aq. TFA unless specified otherwise). Column temperature, 40° C. The components were detected using a UV detector at wavelengths 214 nm or 243 nm. The injection volume was typically 5 μL.

LCMS Method 1: Gradient was 0-0.5 min, 5% B; 0.5-1 min, 5-30% B; 1-5 min, 30-50% B; 5-6 min, 60% B; 6-6.1 min, 50-5% B, 6.1-8 min 5% B; at a flow rate of 0.40 mL/min. The injection volume was 5 μL. The peaks were detected using UV detector at wavelength, 214 and 243 nm.

LCMS Method 2: Gradient was 0-0.5 min, 5% B; 0.5-1 min, 5-40% B; 1-5 min, 40-90% B; 5-6 min, 90% B; 6-6.1 min, 90-5% B, 6.1-8 min 5% B; at a flow rate of 0.40 mL/min. The injection volume was 5 μL. The peaks were detected using UV detector at wavelength, 214 and 243 nm.

LCMS Method 3: Gradient was 0-0.5 min, 5% B; 0.5-1 min, 5-30% B; 1-5 min, 30-70% B; 5-6 min, 70% B; 6-6.1 min, 70-5% B, 6.1-8 min 5% B; at a flow rate of 0.40 mL/min. The injection volume was 5 μL. The peaks were detected using UV detector at wavelength, 214 and 243 nm (Solvent C; 10% of 100 mM aqueous ammonium formate).

LCMS Method 4: Gradient was 0-0.5 min, 5% B; 0.5-1 min, 5-20% B; 1-5 min, 20-90% B; 5-6 min, 90% B; 6-6.1 min, 90-5% B, 6.1-8 min 5% B; at a flow rate of 0.40 mL/min. The injection volume was 5 μL. The peaks were detected using UV detector at wavelength, 214 and 243 nm.

LCMS Method 5: LCMS was recorded using an Agilent ZORBAX 300 Extend-C18 5 μm 4.6×250 mm column with ZORBAX Extent-C18 Guard cartridge 4.6×12.5 mm using a ternary solvent system consisting of solvent A (water), solvent B (acetonitrile) and solvent C (10% of 1% v/v aq. TFA). Column temperature, ambient. The components were detected using a UV detector at wavelengths 214 nm or 243 nm. Gradient was 0-1 min, 0% B; 1-8 min, 0-90% B; 8-10 min 90% B; 10-11 min, 90-0% B; 11-18 min, 0% B. The injection volume was 5 μL. The peaks were detected using UV detector at wavelength, 214 and 243 nm.

### Analytical HPLC

Unless otherwise stated, HPLC were recorded on Waters XBridge™ 3.5 μm 3×100 mm C8 column using a ternary solvent system consisting of solvent A (water), solvent B (acetonitrile) and solvent C (10% of 100 mM aqueous ammonium formate). Column temperature, 50° C. The components were detected using a UV detector at wavelengths 214 nm, 243 nm or 256 nm. The injection volume was typically 5 μL.

HPLC Method 1: Gradient was: 0-1 min, 5% B; 1-7 min, 5-80% B; 7-12 min, 80% B; 12-13 min, 80-5% B; 13-15 min, 5% B; at a flow rate of 0.40 mL/min.

HPLC Method 2: Gradient was: 0-1 min, 30% B; 1-7 min, 30-80% B; 7-12 min, 80% B; 12-13 min, 80-30% B; 13-15 min, 30% B; at a flow rate of 0.40 mL/min.

HPLC Method 3: Gradient was: 0-1 min, 5% A; 1-1.1 min, 5-40% A; 1.1-11 min, 40-90% A; 11-12 min, 90-5% A; 12-15 min, 5% A; at a flow rate of 0.40 mL/min (For HPLC Method 3, HPLC were recorded on Phenomenex Kinetex® 2.6 μm 100 Å 75×2.1 mm C18 column using a binary solvent system consisting of solvent A (0.1% v/v TFA in MeCN) and solvent B (0.1% TFA in water). Column temperature, 40° C. UV detection at 238 nm.)

HPLC Method 4: Gradient was: 0-0.5 min, 5% B; 0.5-3.5 min, 5-80% B; 3.5-6 min, 80% B; 6-6.5 min, 80-5% B; 6.5-8 min, 5% B; at a flow rate of 0.40 mL/min. Phenomenex Kinetex® 2.6 μm 2.1×75 mm C18 column using ternary solvent system consisting of solvent A (water), solvent B (acetonitrile) and solvent C (1% TFA in water v/v). Column temperature, 40° C. The components were detected using a UV detector at wavelengths 214 nm, 243 nm or 254 nm. The injection volume was typically 5 μL.

### Preparative HPLC

Preparative HPLC was performed on Gilson HPLC system using Waters XBridge™ BEH300 Prep C18 5 μm OBD™ 30×150 mm column using a binary solvent system consisting of solvent A and solvent B. The components were detected using a UV detector at the wavelengths stipulated in the method.

Prep-HPLC Method 1: Solvent A, Water; Solvent B, MeCN; flow rate: 8.0 mL/min; gradient: 0-5 min, 45% B; 5-35 min, 45-55% B; 35-40 min, 55-100% B; 40-45 min, 100% B; 45-50 min, 100-45% B, 50-60 min 45% B; Detection at X, =243 nm.

Prep-HPLC Method 2: Solvent A, Water; Solvent B, MeCN; flow rate: 8.0 mL/min; gradient: 0-5 min, 40% B; 5-40 min, 40-70%, 40-45 min, 70% B; 45-50 min, 70-40% B; 50-60 min, 40% Detection at X=243 nm.

Prep-HPLC Method 3: Solvent A, Water; Solvent B, MeCN; flow rate: 8.0 mL/min; gradient: 0-5 min, 40% B; 5-40 min, 40-60%, 40-45 min, 60% B; 45-50 min, 60-40% B; 50-60 min, 40% Detection at X=243 nm.

Prep-HPLC Method 4: Solvent A, 0.05% TFA (v/v) in water; Solvent B, 0.05% TFA (v/v) in MeCN; flow rate: 8.0 mL/min; gradient: 0-5 min, 20% B; 5-40 min, 20-90%, 40-45 min, 90% B; 45-50 min, 90-20% B; 50-60 min, 20% Detection at X, =243 nm.

### SEC

SEC was performed on Sephadex™ LH-20 column under gravity using MeOH or MeCN as the eluent at a flow rate of ˜40-60 drops/min with a fraction size of 400 drops.

### NMR

NMR spectra were recorded on a Bruker (Bruker Daltonics Inc, NSW, Australia) 300 UltraShield™ 300 MHz NMR instrument.

API Loading Method 1: The quantity of Remdesivir RHa-1 loaded onto the dendrimer was determined by ¹H NMR spectroscopy using an internal standard (3,4,5-trichloropyridine). Accurately weighed quantities of both the dendrimer-Remdesivir construct and the internal standard were dissolved in a suitable deuterated solvent and the ¹H NMR spectrum of the solution recorded. By comparing the integrated areas of the internal standard and selected regions of the dendrimer-Remdesivir construct (i.e. areas of the ¹H NMR spectrum attributable only to resonances of Remdesivir), the number of moles of Remdesivir could be calculated per mole of construct.

### Drug Release

Release Method 1: An accurately weighed amount of the Remdesivir-dendrimer (10 mg) construct was dissolved in pH 7.4 PBS buffer:DMSO (9:1 v/v) and the volume made up to 20.0 mL in a volumetric flask to give a 0.5 mg/mL stock solution. The stock solution was aliquoted into several 2 mL screw-cap vials, the caps fitted and the vials heated at 37° C. The aliquoted solutions were analyzed at different time points by HPLC (HPLC Method 3). The concentration of Remdesivir RHa-1 was calculated by comparing the area under the peak associated with Remdesivir RHa-1 to a standard curve in MeCN:DMSO (9:1, v/v). The percentage of Remdesivir RHa-1 released is the percentage of available Remdesivir RHa-1 based on the loading of the construct as determined by ¹H NMR analysis (API Loading Method 1).

### Viscometry

Viscosities were measured on Anton Paar ViscoQC300 Viscometer using CC-12 cup.

Viscosity Method 1: A reference standard solution (Standard Type: RT500, CAS No. 63148-62-9, Paragon Scientific Ltd) was first recorded and found to be within the range of reported value (at 20° C.: found 556.6 cP, theoretical 551.0 cP; at 25° C.: found 503.3 cP, theoretical 498.6 cP). Accurately weighed quantities of respective conjugates were dissolved in EtOH:Water (9:1 v/v) to make a 50 mg/mL Remdesivir equivalent solution (total volume 2.30-2.40 mL) and the viscosity of the solution was recorded at 20° C. and 25° C.

### Synthesis of Intermediates

### 3′O-Glu-Remdesivir RHa-2

To a stirred suspension of Remdesivir RHa-1 (201 mg, 0.33 mmol) (Chemieliva Pharmaceutical Co., Ltd (China)) in DCM (5.0 mL) at 0° C. was added glutaric anhydride (46 mg, 0.40 mmol) followed by NMM (37 μL, 0.33 mmol). After 1 h, the reaction mixture was warmed to rt and stirred for 18 h whereupon DMF (1.5 mL to dissolve any suspended solids that remained) followed by DIPEA (60 μL, 0.33 mmol) and the progress of the reaction followed by HPLC (Method 1). After 1 d, another aliquot of glutaric anhydride (8.0 mg, 0.07 mmol) was added and stirring continued for 2 d whereupon the reaction mixture was concentrated in vacuo. The residue was purified by preparative HPLC (Method 1) to give 3′O-Glu-Remdesivir RHa-2 as a white solid (98 mg, 41%). ¹H NMR (300 MHz, d₆-DMSO): δ (ppm) 1.35-0.98 (m, 7H), 0.80 (t, J=7.4 Hz, 6H), 1.46-1.36 (m, 1H), 1.85-1.75 (m, 2H), 2.37-2.18 (m, 2H), 2.43 (t, J=7.4 Hz, 2H), 4.01-3.69 (m, 3H), 4.31-4.10 (m, 2H), 4.60-4.40 (m, 1H), 5.00 (d, J=5.7 Hz, 1H), 5.27-5.10 (m, 1H), 6.04 (dd, J=13.0, 10.0 Hz, 1H), 7.00-6.82 (m, 2H), 7.19-7.13 (m, 3H), 7.45-7.23 (m, 2H) and 7.94-7.90 (m, 3H), HPLC (HPLC Method 1): Rₜ=8.02 min. LCMS (LCMS Method 1): Rₜ=5.83. ESI MS (+ve) 717 [M]⁺; calc. m/z for C₃₂H₄₁N₆O₁₁P [M]⁺=716.7.

### 6N-TDA-Remdesivir RHa-4

To a solution of Remdesivir RHa-1 (150 mg, 0.25 mmol) in DMF (2.5 mL) at 0° C. was added thiodiglycolic anhydride (66 mg, 0.50 mmol). After 10 min, the reaction mixture was warmed to rt and monitored by HPLC (HPLC Method 2). After 6 d, the reaction mixture was partitioned between DCM (3 mL) and aq pH 3 citrate buffer (2.5 mL). The two-phase mixture was stirred vigorously for 30 min and the organics were separated and washed with more aq pH 3 citrate buffer (3×3.0 mL), dried (MgSO₄) and concentrated in vacuo.

In a separate procedure conducted in parallel to that described above, a solution of Remdesivir RHa-1 (150 mg, 0.25 mmol) in DMF (6.0 mL) at 0° C. was added thiodiglycolic anhydride (66 mg, 0.50 mmol). After 10 min, the reaction mixture was warmed to rt and monitored by HPLC (HPLC Method 2). After 6 d, the reaction mixture was partitioned between DCM (5.0 mL) and aq pH 3 citrate buffer (5.0 mL). The two-phase mixture was stirred vigorously for 50 min and the organics were separated and washed with more aq pH 3 citrate buffer (3×3.0 mL), dried (MgSO₄) and concentrated in vacuo.

Both residues described above were dissolved MeCN (5.0 mL) and combined and concentrated in vacuo. The residue was dissolved in DCM (10 mL) and washed with aq pH 3 citrate buffer (2×5.0 mL), dried (MgSO₄), filtered (0.45 μm porosity syringe filter disc) and concentrated in vacuo to give 6N-TDA-Remdesivir RHa-4 a pale yellow oil (313 mg). The material was used without further purification (purity ˜60% by ¹H NMR analysis (contains ˜30% Remdesivir RHa-1 and other uncharacterized impurities)). ¹H NMR (300 MHz, d₆-DMSO): δ (ppm) 0.79 (t, J=7.4 Hz, 6H), 1.08-1.33 (m, 7H), 1.32-1.51 (m, 1H), 3.41 (s, 2H), 3.66-4.02 (m, 6H), 4.01-4.17 (m, 1H), 4.16-4.37 (m, 2H), 4.55-4.70 (m, 1H), 5.30-5.49 (m, 1H), 6.03 (dd, J=13.0, 10.0 Hz, 1H), 6.42 (d, J=6 Hz, 1H), 7.02-7.24 (m, 4H), 7.24-7.44 (m, 3H), 8.39 (s, 1H), 11.1 (br, 1H), 12.64 (br, 1H); HPLC (HPLC Method 1): Rₜ=8.34 min. LCMS (LCMS Method 2): Rₜ=6.20. ESI MS (+ve) 735 [M]⁺; calc. m/z for C₃₁H₃₉N₆O₁₁PS [M]⁺=734.7.

### 6N-DMF-Remdesivir RHa-7

To a solution of Remdesivir RHa-1 (630 mg, 1.05 mmol) in DMF (5.0 mL) was added N,N-dimethylformamide dimethyl acetal (278 μL, 2.10 mmol) at rt (N.B. the reaction mixture turned yellow 5 min after the addition of added N,N-dimethylformamide dimethyl acetal). After stirring for 16 h, the reaction mixture was diluted with DCM (10 mL) and concentrated in vacuo. The crude material was combined with another batch (from 607 mg Remdesivir RHa-1, 1.01 mmol) and purified by column chromatography on silica gel eluting with DCM:MeOH [gradient elution from 100:0 (v/v) to 95:5 (v/v)] to give 6N-DMF-Remdesivir RHa-7 [0.86 g, 63% (calculated based on combined yield)]. ¹H NMR (300 MHz, d₆-DMSO): δ (ppm) 0.79 (t, J=6.0 Hz, 6H), 1.07-1.34 (m, 7H), 1.34-1.51 (m, 1H), 3.18 (s, 3H), 3.25 (s, 3H), 3.58-4.18 (m, 5H), 4.18-4.36 (m, 2H), 4.66-4.70 (m, 1H), 5.38 (d, J=4.0 Hz, 1H), 6.00 (dd, J=15.0, 12.0 Hz, 1H), 6.31 (d, J=4.0 Hz, 1H), 6.80 (d, J=4.0 Hz, 1H), 6.93 (d, J=4.0 Hz, 1H), 7.14-7.20 (m, 3H), 7.31-7.36 (m, 2H), 8.14 (s, 1H), 8.94 (s, 1H); HPLC (HPLC Method 1): Rₜ=8.94 min.

### 3′ O-DGA-Remdesivir RHa-10

To a stirred solution of 6N-DMF-Remdesivir RHa-7 (260 mg, 0.40 mmol) in DCM (8.0 mL) at 0° C. was added diglycolic anhydride (69 mg, 0.59 mmol) followed by NMM (53 μL, 0.47 mmol). The reaction mixture was stirred for 1 h and then allowed to warm to rt. After 20 h, the reaction mixture was diluted with DCM (20 mL) and the organics were shaken with pH 3 citrate buffer (20 mL). The pH of the aqueous layer checked and adjusted back to pH 3 by dropwise addition of 1.0M HCl (aq) if required. The organic phase was separated and washed again with pH 3 buffer (3×20 mL), brine (20 mL) and dried (MgSO₄). The volatiles were removed in vacuo to give a mixture of 6N-DMF-3′O-DGA-Remdesivir RHa-8 and 6N-DMF-2′O,3′O-bis-DGA-Remdesivir RHa-9 (320 mg). The crude mixture (215 mg) was dissolved in MeCN:water (6.0 mL, 2:1 v/v) and heated with stirring at 60° C. for 3 h and then cooled to rt and stirred for 19 h whereupon formic acid (1.0 mL) was added and stirring continued. After stirring for an additional 23 h, the reaction mixture was concentrated in vacuo and the residue and purified by preparative HPLC (prep-HPLC Method 2) to give 3′ O-DGA-Remdesivir RHa-10 as an off-white solid (95 mg, ˜70-75% pure by ¹H NMR spectroscopy) and Remdesivir RHa-1 (48 mg). ¹H NMR (300 MHz, d₆-DMSO): δ (ppm) 0.80 (t, J=7.4 Hz, 6H), 1.08-1.33 (m, 7H), 1.33 (m, 1H), 3.64-4.01 (m, 3H), 4.01-4.42 (m, 6H), 4.42-4.54 (m, 1H), 5.02 (t, J=5.3 Hz, 1H), 5.17-5.30 (m, 1H), 6.05 (dd, J=13.0, 10.0 Hz, 1H), 6.67 (d, J=18.0 Hz, 1H), 6.74-6.96 (m, 2H), 7.05-7.23 (m, 3H), 7.23-7.43 (m, 2H), 7.64-8.21 (br, 3H). HPLC (HPLC Method 1): Rₜ=7.53 min. LCMS (LCMS Method 3): Rₜ=4.15 min. ESI MS (+ve) 719 [M]⁺; calc. m/z for C₃₁H₃₉N₆O₁₂P [M]⁺=718.7.

### 3′O-TDA-Remdesivir RHa-14

To a stirred solution of 6N-DMF-Remdesivir RHa-7 (130 mg, 0.20 mmol) in DCM (5.2 mL) at 0° C. was added DIPEA (42 μL, 0.24 mmol) followed by thiodiglycolic anhydride (40 mg, 0.30 mmol). The reaction mixture was stirred for 10 min and then allowed to warm to rt. After 45 min the reaction mixture was diluted with DCM (20 mL) and the organics were shaken with pH 3 citrate buffer (20 mL). The pH of the aqueous layer checked and adjusted back to pH 3 by dropwise addition of 1.0M HCl(aq) if required. The organic phase was separated and washed again with pH 3 buffer (20 mL) and dried (MgSO₄). The volatiles were removed in vacuo to give a mixture of 6N-DMF-3′O-TDA-Remdesivir RHa-12 and 6N-DMF-2′O,3′ O-bis-TDA-Remdesivir RHa-13 (174 mg). The crude mixture was dissolved in MeCN:water (4.5 mL, 4:5 v/v) and stirred at rt for 19 h and then heated at 70° C. for 5 h. The reaction mixture was then cooled to rt whereupon formic acid (2.0 mL) was added and stirring continued. After 16 h, the reaction mixture was concentrated in vacuo and the residue was purified by preparative HPLC (prep-HPLC Method 3) to give 3′ O-TDA-Remdesivir RHa-14 as a white solid (58 mg). ¹H NMR (300 MHz, d₆-DMSO): δ (ppm) 0.80 (t, J=7.7 Hz, 6H), 1.09-1.32 (m, 7H), 1.32-1.56 (m, 1H), 3.34 (s, 2H), 3.54 (s, 2H), 3.67-4.04 (m, 3H), 4.04-4.37 (m, 2H), 4.37-4.56 (m, 1H), 4.59-4.66 (m, 1H), 5.00 (d, J=5.4 Hz, 1H), 5.09-5.27 (m, 1H), 6.04 (dd, J=13.0, 10.0 Hz, 1H), 6.80-6.95 (m, 2H), 7.05-7.23 (m, 3H), 7.23-7.37 (m, 2H), 7.70-8.10 (m, 3H). HPLC (HPLC Method 1): Rₜ=7.66 min.

### GS-441524 Intermediates

### 2′O—, 3′O-Acetonide-GS-441524 RL-2

To a stirred suspension of GS-441524 RL-1 (295 mg, 1.01 mmol) and 2,2-dimethoxypropane (622 μL, 5.07 mmol) in acetone (2.0 mL) was added conc. sulphuric acid (98%, 67 μL, 1.21 mmol) at rt. The resulting solution was stirred for 10 min and then heated at 50° C. After 30 min, the reaction mixture was cooled to rt and stirred for 18 h whereupon sodium bicarbonate (325 mg, 3.07 mmol) was added followed by water (320 μL). The reaction mixture was stirred for 30 min and then concentrated in vacuo. Water (10 mL) was added to the residue and the organics were extracted into EtOAc (2×10 mL). The combined organics were dried washed with brine (20 mL), dried (MgSO₄) and concentrated in vacuo to give 2′ O-, 3′ O-acetonide-GS-441524 RL-2 as an off-white foam (640 mg), which was used without further purification.

¹H NMR (300 MHz, d₆-DMSO): δ (ppm) 1.37 (s, 3H), 1.63 (s, 3H), 3.48-3.61 (m, 2H), 4.27-4.35 (m, 1H), 4.86-4.92 (m, 1H), 5.01 (t, J=5.6 Hz, 1H), 5.39 (d, J=6.3 Hz, 1H), 6.86-6.94 (m, 2H) and 7.81-8.07 (brm, 3H).

### 2′O-, 3′O-Acetonide-6N-formamidine-GS-441524 RL-3

To a solution of crude 2′ O-, 3′ O-Acetonide-GS-441524 RL-2 (331 mg, 1.01 mmol) in DMF (10 mL) was added N,N-dimethylformamide dimethyl acetal (270 μL, 2.02 mmol). The reaction mixture was stirred for 18 h and then concentrated in vacuo. The residue was dissolved in DCM (10 mL), washed with water (5 mL), brine (5 mL) and dried (MgSO₄). The volatiles were removed in vacuo to give 2′ O-, 3′ O-Acetonide-6N-formamidine-GS-441524 RL-3 as an off-white foam (541 mg).

¹H NMR (300 MHz, d₆-DMSO): δ (ppm) 1.41 (s, 3H), 1.73 (s, 3H), 3.24 (s, 6H), 3.67-3.87 (m, 3H), 4.47-4.57 (m, 1H), 5.08 (dd, J=6.6 and 3.1 Hz, 1H), 5.41 (d, J=6.4 Hz, 1H), 6.94 (dd, J=4.5 Hz, 2H), 8.05 (s, 1H) and 8.92 (s, 1H). LCMS (LCMS Method 4): Rₜ=4.04. ESI MS (+ve) 387 NW; calc. m/z for C₁₈H₂₃N₆O₄[MH]⁺=387.

### 5′O-DGA-GS-441524 RL-4

To a solution of 2′O-, 3′ O-acetonide-6N-formamidine-GS-441524 RL-3 (265 mg, 0.69 mmol) in DCM (3 mL) at rt was added diglycolic anhydride (95 mg, 0.82 mmol) and DIPEA (240 μL, 1.37 mmol). The reaction mixture was stirred for 18 h and diluted with DCM (15 mL). The organics were shaken with pH 3 phosphate buffer (3×5 mL), brine (5 mL) and dried (MgSO₄). The volatiles were removed in vacuo and the residue was dissolved in MeCN:Water (5 mL, 3:2 v/v) and stirred at rt and monitored by LCMS (Method 4). After 5 d, the reaction mixture was concentrated in vacuo, dissolved in 80% formic acid in water and stirred at rt for 2 d and then concentrated in vacuo. The residue was dissolved in water (˜5 mL) containing a few drops of formic acid and the solution was lyophilised to give 5′ O-DGA-GS-441524 RL-4 as a light brown solid (114 mg, 41%).

¹H NMR (300 MHz, d₃-CD₃CN): δ (ppm) 4.12-4.25 (m, 4H), 4.26-4.54 (m, 4H), 4.73 (d, J=4.8 Hz, 1H), 6.25-6.37 (brm, 2H), 7.10 (d, J=4.8 Hz, 1H), 7.42 (d, J=4.8 Hz, 1H), 8.02-8.08 (m, 1H), 8.26 (brs, 1H) and 8.44 (brs, 1H). LCMS (LCMS Method 5): Rₜ=6.00 min. ESI MS (+ve) 408 [MH]⁺; calc. m/z for C₁₆H₁₇N₅O₈[MH]⁺=408.

### 2′O-,3′O-Acetonide-5′O-TDA-GS-441524 RL-5

To a solution of 2′O, 3′ O-acetonide-6N-formamidine-GS-441524 RL-3 (262 mg, 0.68 mmol) in DCM (5 mL) at rt was added thiodiglycolic anhydride (107 mg, 0.81 mmol) and DIPEA (242 lit, 1.36 mmol). The reaction mixture was stirred for 18 h whereupon another portion of thiodiglycolic anhydride (50 mg, 0.38 mmol) was added. After stirring for an additional 18 h, the reaction mixture was diluted with DCM (10 mL) and the organics were washed with pH 3 phosphate buffer (3×5 mL), brine (5 mL) and dried (MgSO₄). The volatiles were removed in vacuo and the residue was dissolved in MeCN:Water (3 mL, 3:2 v/v) and stirred at rt. After 4d, the reaction mixture was concentrated in vacuo. This material was combined with another batch that was prepared in a similar way to that described above starting from 2′ O-,3′ O-acetonide-6N-formamidine-GS-441524 RL-3 (262 mg, 0.68 mmol), thiodiglycolic anhydride (107 mg, 0.81 mmol) and DIPEA (242 μL, 1.36 mmol). The combined batches were purified by preparative HPLC (Method 4, Rₜ=22 min) to give a yellow oil. The material was dissolved in EtOAc (10 mL) and the organic phase was washed with pH 3 phosphate buffer (3×5 mL), brine (5 mL), dried (MgSO₄) and concentrated in vacuo to give 2′ O-,3′O-acetonide-5′ O-TDA-GS-441524 RL-5 as an off white solid (44 mg, 11%).

¹H NMR (300 MHz, d₃-CD₃CN): δ (ppm) 1.42 (s, 3H), 1.72 (s, 3H), 3.27 (s, 2H), 3.31 (s, 2H), 4.20-4.41 (m, 2H), 4.66-4.68 (m, 1H), 5.00 (dd, J=6.2 and 2.6 Hz, 1H), 5.42 (d, J=6.2 Hz, 1H), 6.61-7.20 (brm, 6H) 7.92 (s, 1H).

### 5′ O-TDA-GS-441524 RL-6

2′ O—,3′ O-Acetonide-5′O-TDA-GS-441524 RL-5 (44 mg, 0.095 mmol) was dissolved in 80% formic acid in water and stirred at rt for 18 h and then concentrated in vacuo to give 5′O-TDA-GS-441524 RL-6 as a light brown oil (40 mg, quant). LCMS (LCMS Method 5): R=6.64 min. ESI MS (+ve) 424 [MH]⁺; calc. m/z for C₁₆H₁₇N₅O₇S [MH]⁺=424.

### Dendrimeric Intermediates

32‡ relates to the number of E surface amino groups on the dendrimer available for substitution with PEG. The actual mean number of PEG groups attached to the BHALys [Lys]₂[Lys]₄[Lys]₈[Lys]₁₆[Lys]₃₂ motif was determined experimentally by ¹H NMR.

Dendrimer intermediates may for example be prepared as described below, and/or as described in WO2012/167309A1.

### BHALys[(α-NHBoc)(ε-NHBoc)]

Solid α,ε-(t-Boc)₂-(L)-lysine p-nitrophenol ester (2.787 kg, 5.96 mol) was added to a solution of aminodiphenylmethane (benzhydrylamine) (0.99 kg, 5.4 mol) in anhydrous acetonitrile (4.0 L), DMF (1.0 L) and triethylamine (1.09 kg) over a period of 15 min. The reaction mixture was agitated at 20° C. overnight. The reaction mixture was then warmed to 35° C. and aqueous sodium hydroxide (0.5 N, 10 L) was added slowly over 30 min. The mixture was stirred for an additional 30 min then filtered. The solid cake was washed with water and dried to a constant weight (2.76 kg, 5.4 mol) in 100% yield. ¹H NMR (300 MHz, CD₃OD) δ 7.3 (m, 10H, Ph Calc. 10H); 6.2 (s, 1H, CH-Ph₂ Calc. 1H); 4.08 (m, α-CH, 1H), 3.18 (br, ε—CH₂) and 2.99 (m, ε—CH₂ 2H); 1.7-1.2 (br, β,γ,δ-CH₂) and 1.43 (s, tBu) total for β,γ,δ-CH₂ and tBu 25H Calc. 24H. MS (ESI+ve) found 534.2 [M+Na]⁺ calc. for C₂₉H₄₁N₃O₅Na [M+Na]⁺534.7.

BHALys[(α-NH₂·HCl)(ε-NH₂·HCl)]

A solution of concentrated HCl (1.5 L) in methanol (1.5 L) was added slowly, in three portions, to a stirred suspension of BHALys[(α-NHBoc)(ε-NHBoc)] (780.5 g, 1.52 mol) in methanol (1.5 L) at a rate to minimize excessive frothing. The reaction mixture was stirred for an additional 30 min, then concentrated under vacuum at 35° C. The residue was taken up in water (3.4 L) and concentrated under vacuum at 35° C. twice, then stored under vacuum overnight. Acetonitrile (3.4 L) was then added and the residue was again concentrated under vacuum at 35° C. to give BHALys[(α-NH₂·HCl)(ε-NH₂·HCl)] as a white solid (586 g, 1.52 mol) in 100% yield. ¹H NMR (300 MHz, D₂O) δ 7.23 (br m, 10H, Ph Calc. 10H); 5.99 (s, 1H, CH-Ph₂ Calc. 1H); 3.92 (t, J=6.5 Hz, α-CH, 1H, Calc. 1H); 2.71 (t, J=7.8 Hz, ε—CH₂, 2H, Calc. 2H); 1.78 (m, β,γ,δ-CH₂, 2H), 1.47 (m, β,γ,δ-CH₂, 2H), and 1.17 (m, β,γ,δ-CH₂, 2H, total 6H Calc. 6H). MS (ESI+ve) found 312 [M+H]⁺ calc. for C₁₉H₂₆N₃O [M+H]⁺ 312.

BHALys[Lys]₂[(α-NHBoc)₂(ε-NHBoc)₂]

To a suspension of BHALys[(α-NH₂·HCl)(ε-NH₂·HCl)] (586 g, 1.52 mmol) in anhydrous DMF (3.8 L) was added triethylamine (1.08 kg) slowly to maintain the reaction temperature below 30° C. Solid α,ε-(t-Boc)₂-(L)-lysine p-nitrophenol ester (1.49 kg) was added in three portions, slowly and with stirring for 2 hours between additions. The reaction was allowed to stir overnight. An aqueous solution of sodium hydroxide (0.5 M, 17 L) was added slowly to the well stirred mixture and stirring was maintained until the solid precipitate was freely moving. The precipitate was collected by filtration, and the solid cake was washed well with water (2×4 L) then acetone/water (1:4, 2×4 L). The solid was slurried again with water then filtered and dried under vacuum overnight to give BHALys[Lys]₂[(α-NHBoc)₂(ε-NHBoc)₂] (1.51 kg) in 100% yield. ¹H NMR (300 MHz, CD₃OD) δ 7.3 (m, 10H, Ph Calc. 10H); 6.2 (s, 1H, CH-Ph₂ Calc. 1H); 4.21 (m, α-CH), 4.02 (m, α-CH) and 3.93 (m, α-CH, total 3H, Calc. 3H); 3.15 (m, ε—CH₂) and 3.00 (m, ε—CH₂ total 6H, Calc. 6H); 1.7-1.3 (br, β,γ,δ-CH₂) and 1.43 (s, tBu) total for β,γ,δ-CH₂ and tBu 57H, Calc. 54H. MS (ESI+ve) found 868.6 [M−Boc]⁺; 990.7 [M+Na]⁺ calc. for C₅₁H₈₁N₇O₁₁Na [M+Na]⁺991.1.

BHALys[Lys]₂[(α-NH₂·HCl)₂(ε-NH₂HCl)₂]

BHALys[Lys]₂[(α-NHBoc)₂(ε-NHBoc)₂] (1.41 kg, 1.46 mol) was suspended in methanol (1.7 L) with agitation at 35° C. Hydrochloric acid (1.7 L) was mixed with methanol (1.7 L), and the resulting solution was added in four portions to the dendrimer suspension and left to stir for 30 min. The solvent was removed under reduced pressure and worked up with two successive water (3.5 L) strips followed by two successive acetonitrile (4 L) strips to give BHALys[Lys]₂[(α-NH₂·HCl)₂(ε-NH₂HCl)₂] (1.05 Kg, 1.46 mmol) in 102% yield. ¹H NMR (300 MHz, D₂O) δ 7.4 (br m, 10H, Ph Calc. 10H); 6.14 (s, 1H, CH-Ph₂ Calc. 1H); 4.47 (t, J=7.5 Hz, α-CH, 1H), 4.04 (t, J=6.5 Hz, α-CH, 1H), 3.91 (t, J=6.8 Hz, α-CH, 1H, total 3H, Calc. 3H); 3.21 (t, J=7.4 Hz, ε—CH₂, 2H), 3.01 (t, J=7.8 Hz, ε—CH₂, 2H) and 2.74 (t, J=7.8 Hz, ε—CH₂, 2H, total 6H, Calc. 6H); 1.88 (m, β,γ,δ-CH₂), 1.71 (m, β,γ,δ-CH₂), 1.57 (m, β,γ,δ-CH₂) and 1.35 (m, β,γ,δ-CH₂ total 19H, Calc. 18H).

BHALys[Lys]₂[Lys]₄[(α-NHBoc)₄(ε-NHBoc)₄]

BHALys[Lys]₂[HCl]₄ (1.05 Kg, 1.47 mol) was dissolved in DMF (5.6 L) and triethylamine (2.19 L). The α,ε-(t-Boc)₂-(L)-lysine p-nitrophenol ester (2.35 kg, 5.03 mol) was added in three portions and the reaction stirred overnight at 25° C. A NaOH (0.5M, 22 L) solution was added and the resulting mixture filtered, washed with water (42 L) and then air dried. The solid was dried under vacuum at 45° C. to give BHALys[Lys]₂[Lys]₄[(α-NHBoc)₄(ε-NHBoc)₄] (2.09 Kg, 1.11 mol) in 76% yield. ¹H NMR (300 MHz, CD₃OD) δ 7.3 (m, 10H, Ph Calc. 10H); 6.2 (s, 1H, CH-Ph₂ Calc. 1H); 4.43 (m, α-CH), 4.34 (m, α-CH), 4.25 (m, α-CH) and 3.98 (br, α-CH, total 7H, Calc. 7H); 3.15 (br, ε—CH₂) and 3.02 (br, ε—CH₂ total 14H, Calc. 14H); 1.9-1.2 (br, β,γ,δ-CH₂) and 1.44 (br s, tBu) total for β,γ,δ-CH₂ and tBu 122H, Calc. 144H.

BHALys[Lys]₂[Lys]₄[(α-NH₂·TFA)₄(ε-NH₂·TFA)₄]

To a stirred suspension of BHALys[Lys]₂[Lys]₄[(α-NHBoc)₄(ε-NHBoc)₄] (4 g, 2.13 mmol) in DCM (18 mL) was added TFA (13 mL) at 0° C. The solids dissolved, and the solution was stirred overnight under an atmosphere of argon. The solvents were removed under vacuum, and residual TFA was removed by trituration with diethyl ether (100 mL). The product was redissolved in water then freeze dried to give BHALys[Lys]₂[Lys]₄[(α-NH₂·TFA)₄(ε-NH₂·TFA)₄] as an off-white solid (4.27 g, 2.14 mmol) in 101% yield. ¹H NMR (300 MHz, D₂O) δ 7.21 (br m, 10H, Ph Calc. 10H); 5.91 (s, 1H, CH-Ph₂ Calc. 1H); 4.17 (t, J=7.4 Hz, α-CH, 1H), 4.09 (t, J=7.1 Hz, α-CH, 1H), 4.02 (t, J=7.2 Hz, α-CH, 1H, 3.84 (t, J=6.5 Hz, α-CH, 2H), 3.73 (t, J=6.7 Hz, α-CH, 1H), 3.67 (t, J=6.7 Hz, α-CH, 1H, total 7H, Calc. 7H); 3.0 (m, ε—CH₂), 2.93 (m, ε—CH₂) and 2.79 (b, ε—CH₂, total 15H, Calc. 14H); 1.7 (br, β,γ,δ-CH₂), 1.5 (br, β,γ,δ-CH₂), 1.57 (m, β,γ,δ-CH₂) and 1.25 (br, β,γ,δ-CH₂ total 45H, Calc. 42H). MS (ESI+ve) found 541.4 [M+2H]²⁺; calc. for C₅₅H₉₉N₁₅O₇ [M+2H]²⁺ 541.2.

BHALys[Lys]₂[Lys]₄[Lys]₈[(α-NHBoc)₈(ε-NHBoc)₈]

A solution of α,ε-(t-Boc)₂-(L)-lysine p-nitrophenol ester (1.89 g, 4.05 mmol) in DMF (25 mL) was added to a solution of BHALys[Lys]₂[Lys]₄[(α-NH₂·TFA)₄(ε-NH₂·TFA)₄] (644 mg, 0.32 mmol) and triethylamine (0.72 mL, 5.2 mmol) in DMF (25 mL) and the reaction was left to stir overnight under an argon atmosphere. The reaction mixture was poured onto ice/water (500 mL) then filtered and the collected solid was dried overnight under vacuum. The dried solid was washed thoroughly with acetonitrile to give BHALys[Lys]₂[Lys]₄[Lys]₈[(α-NHBoc)₈(ε-NHBoc)₈] as an off white solid (0.82 g, 0.22 mmol) in 68% yield. ¹H NMR (300 MHz, CD₃OD) δ 7.3 (m, 10H, Ph Calc. 10H); 6.2 (br s, 1H, CH-Ph₂ Calc. 1H); 4.48 (br, α-CH), 4.30 (br, α-CH) and 4.05 (br, α-CH, total 16H Calc. 15H); 3.18 (br, ε—CH₂) and 3.02 (m, ε—CH₂ total 31H, Calc. 30H); 1.9-1.4 (br, β,γ,δ-CH₂) and 1.47 (br s, tBu) total for β,γ,δ-CH₂ and tBu 240H, Calc 234H. MS (ESI+ve) found 3509 [M+H−(Boc)₂]⁺ calc. for C₁₇₃H₃₀₆N₃₁O₄₃ [M+H−(Boc)₂]⁺ 3508.5; 3408 [M+H−(Boc)₃]⁺ calc. for C₁₆₈H₂₉₈N₃₁O₄₁ [M+H−(Boc)₃]⁺ 3408.4.

BHALys[Lys]₂[Lys]₄[Lys]₈[(α-NH₂·TFA)₈(ε-NH₂·TFA)₈]

A solution of TFA/DCM (1:1, 19 mL) was added slowly to a stirred suspension of BHALys[Lys]₂[Lys]₄[Lys]₈[(α-NHBoc)₈(ε-NHBoc)₈] (800 mg, 0.22 mmol) in DCM (25 mL). The solids dissolved, and the solution was stirred overnight under an atmosphere of argon. The solvents were removed under vacuum, and residual TFA was removed by repetitive freeze drying of the residue, to give BHALys[Lys]₂[Lys]₄[Lys]₈[(α-NH₂·TFA)₈(ε-NH₂·TFA)₈] as an off-white lyophilisate (848 mg, 0.22 mmol) in 100% yield. ¹H NMR (300 MHz, D₂O) δ 7.3 (br m, 10H, Ph Calc. 10H); 6.08 (s, 1H, CH-Ph₂ Calc. 1H); 4.3 (m, α-CH), 4.18 (m, α-CH), 4.0 (m, α-CH) and 3.89 (m, α-CH, total 16H, Calc. 15H); 3.18 (br, ε—CH₂) and 2.94 (m, ε—CH₂ total 32H, Calc. 30H); 1.9 (m, β,γ,δ-CH₂), 1.68 (m, β,γ,δ-CH₂) and 1.4 (m, β,γ,δ-CH₂ total 99H, Calc. 90H). MS (ESI+ve) found 2106 [M+H]⁺ calc. for C₁₀₃H₁₉₄N₃₁O₁₅ [M+H]⁺ 2106.9.

BHALys[Lys]₂[Lys]₄[Lys]₈[Lys]₁₆[(α-NHBoc)₁₆(ε-NHBoc)₁₆]

A solution of α,ε-(t-Boc)₂-(L)-lysine p-nitrophenol ester (1.89 g, 4.05 mmol) in DMF (25 mL) was added to a solution of BHALys[Lys]₂[Lys]₄[Lys]₈[(α-NH₂·TFA)₈(ε-NH₂·TFA)₈] (644 mg, 0.32 mmol) and triethylamine (0.72 mL, 5.2 mmol) in DMF (25 mL) and the reaction was left to stir overnight under an argon atmosphere. The reaction was poured onto ice/water (500 mL) then filtered and the collected solid was dried overnight under vacuum. The dried solid was washed thoroughly with acetonitrile to give BHALys[Lys]₂[Lys]₄[Lys]₈[Lys]₁₆[(α-NHBoc)₁₆(ε-NHBoc)₁₆] as an off white solid (0.82 g, 0.2 2 mmol) in 68% yield. ¹H NMR (300 MHz, CD₃OD) δ 7.28 (m, 9H, Ph Calc. 10H); 6.2 (br s, 1H, CH-Ph₂ Calc. 1H); 4.53 (br, α-CH), 4.32 (br, α-CH) and 4.05 (br, α-CH, total 35H, Calc. 31H); 3.18 (br, ε—CH₂) and 3.04 (m, ε—CH₂ total 67H, Calc. 62H); 1.9-1.5 (br, β,γ,δ-CH₂) and 1.47 (br s, tBu) total for β,γ,δ-CH₂ and tBu 474H Calc, 474H. MS (ESI+ve) found 6963 [M+H−(Boc)₄]⁺ calc for C₃₃₉H₆₁₀N₆₃O₈₇ [M+H−(Boc)₄]⁺ 6960.9; 6862 [M+H−(Boc)₅]⁺ calc. for C₃₃₄H₆₀₄N₆₃O₈₅ [M+H−(BOC)₅]⁺ 6860.8.

BHALys[Lys]₂[Lys]₄[Lys]₈[Lys]₁₆[(α-NH₂·TFA)₁₆(ε-NH₂·TFA)₁₆]

A solution of TFA/DCM (1:1, 19 mL) was added slowly to a stirred suspension of BHALys[Lys]₂[Lys]₄[Lys]₈[Lys]₁₆[(α-NHBoc)₁₆(ε-NHBoc)₁₆] (800 mg, 0.11 mmol) in DCM (25 mL). The solids dissolved, and the solution was stirred overnight under an atmosphere of argon. The solvents were removed under vacuum, and residual TFA was removed by repetitive freeze drying of the residue, to give BHALys [Lys]₂[Lys]₄[Lys]₈[Lys]₁₆[(α-NH₂·TFA)₁₆(ε-NH₂·TFA)₁₆] as an off-white lyophilisate (847 mg, 0.11 mmol) in 100% yield. ¹H NMR (300 MHz, D₂O) δ 7.3 (br m, 11H, Ph Calc. 10H); 6.06 (s, 1H, CH-Ph₂ Calc. 1H); 4.3 (m, α-CH), 4.19 (m, α-CH), 4.0 (m, α-CH) and 3.88 (m, α-CH, total 35H, Calc. 31H); 3.15 (br, ε—CH₂) and 2.98 (m, ε—CH₂ total 69H, Calc 62H); 1.88 (m, β,γ,δ-CH₂), 1.7 (m, β,γ,δ-CH₂) and 1.42 (m, β,γ,δ-CH₂ total 215H, Calc. 186H). MS (ESI+ve) found 4158 [1\4+H]⁺ calc. for C₁₉₉H₃₈₆N₆₃O₃₁ [M+H]⁺ 4157.6

### HO-Lys(α-Boc)(ε-PEG_{˜2000})

DIPEA (0.37 mL, 2.10 mmol) was added to an ice-cooled mixture of NHS-PEG_{˜2100} (2.29 g, 1.05 mmol) (in which PEG_{˜2000} represents a methoxy-terminated PEG group having approximate average molecular weight of 2000 Da, and in which NHS represents NHS—C(O)CH₂), and N-α-t-BOC-L-lysine (0.26 g, 1.05 mmol) in DMF (20 mL). The stirred mixture was allowed to warm to rt overnight then any remaining solids were filtered (0.45 μm PALL acrodisc) before removing the solvent in vacuo. The residue was taken up in MeCN:H₂O (1:3, 54 mL) and purified by PREP HPLC (Waters XBridge C18, 5 μm, 19×150 mm, 25 to 32% MeCN (5-15 min), 32 to 60% MeCN (15 to 20 min), no buffer, 8 mL/min, Rₜ=17 min), providing 1.41 g (56%) of HO-Lys(α-Boc(ε-PEG_{˜2000}). ¹H NMR (300 MHz, CD₃OD) δ 3.96-4.09 (m, 1H), 3.34-3.87 (m, 188H); 3.32 (s, 3H), 3.15 (q, J=6.0 Hz, 2H), 2.40 (t, J=6.2 Hz, 2H), 1.28-1.88 (m, 6H), 1.41 (s, 9H).

BHALys[Lys]₂[Lys]₄[Lys]₈[Lys]₁₆[Lys]₃₂[α-NHBoc]₃₂[ε-NH-COPEG₂₀₀₀]_{32‡}

To a stirred mixture of BHALys[Lys]₂[Lys]₄[Lys]₈[Lys]₁₆[(α-NH₂·TFA)₁₆(ε-NH₂·TFA)₁₆] (0.19 g, 24 mol) in DMF (20 mL) was added DIPEA (0.86 mL, 4.86 mmol). This mixture was then added dropwise to a stirred mixture of PyBOP (0.62 g, 1.20 mmol) and HO-Lys(α-Boc)(ε-PEG_{˜2000}) (2.94 g, 1.20 mmol) in DMF (20 mL) at room temperature. The reaction mixture was left to stir overnight, then diluted with water (200 mL). The aqueous mixture was subjected to a centramate filtration (5 k membrane, 20 L water). The retentate was freeze dried, providing 1.27 g (73%) of desired dendrimer. HPLC (C8 XBridge, 3×100 mm, gradient: 5% MeCN (0-1 min), 5-80% MeCN/H₂O) (1-7 min), 80% MeCN (7-12 min), 80-5% MeCN (12-13 min), 5% MeCN (13-15 min), 214 nm, 0.1% TFA) Rₜ (min)=8.52. ¹H NMR (300 MHz, D₂O) δ 1.10-2.10 (m, Lys CH₂ (β, χ, δ) and BOC, 666H), 3.02-3.36 (m, Lys CH₂ (ε), 110H), 3.40 (s, PEG-OMe, 98H), 3.40-4.20 (m, PEG-OCH₂, 5750H+Lys CH surface, 32H), 4.20-4.50 (m, Lys, CH internal 32H), 7.20-7.54 (m, BHA, 8H). ¹H NMR indicates approximately 29 PEGs.

BHALys[Lys]₂[Lys]₄[Lys]₈[Lys]₁₆[Lys]₃₂[α-NH₂·TFA]₃₂[ε-NH-COPEG₂₀₀₀]_{32‡} RHa-6

1.27 g (17.4 μmol) of BHALys[Lys]₂[Lys]₄[Lys]₈[Lys]₁₆[Lys]₃₂[α-NHBoc]₃₂[ε-NHCOPEG₂₀₀₀]_{32‡} was stirred in TFA/DCM (1:1, 20 mL) at room temperature overnight. The volatiles were removed in vacuo, then the residue was taken up in water (30 mL). The mixture was then concentrated. This process was repeated two more times before being freeze dried, providing 1.35 g (106%) of desired product as a viscous colourless oil. HPLC (C8 XBridge, 3×100 mm, gradient: 5% MeCN (0-1 min), 5-80% MeCN/H₂O) (1-7 min), 80% MeCN (7-12 min), 80-5% MeCN (12-13 min), 5% MeCN (13-15 min), 214 nm, 0.1% TFA) Rₜ (min)=8.51. ¹H NMR (300 MHz, D₂O) δ 1.22-2.08 (Lys CH₂ ((β, χ, δ), 378H), 3.00-3.26 (Lys CH₂ (ε), 129H), 3.40 (PEG-OMe, 96H), 3.45-4.18 (PEG-OCH₂, 5610H+Lys CH surface, 32H), 4.20-4.46 (Lys, CH internal, 33H), 7.24-7.48 (8H, BHA). ¹H NMR indicates approximately 29 PEGs.

BHALys[Lys]₂[Lys]₄[Lys]₈[Lys]₁₆[Lys]₃₂[α-NH₂·TFA]₃₂[ε-NH-COPEG₁₀₀₀]_{32‡} RHa-16

The title compound was prepared in an analogous fashion to BHALys [Lys]₂[Lys]₄[Lys]₈[Lys]₁₆[Lys]₃₂[α-NH₂·TFA]₃₂[ε-NHCOPEG₂₀₀₀]_{32‡} as described above, but using NHS-PEG_{˜1000} in place of NHS-PEG_{˜2000}. ¹H NMR (300 MHz, MeOD) δ 8.12-8.01 (m, 21H), 7.38-7.30 (m, 13H), 6.09 (s, 3H), 4.35 (s, 39H), 4.04-3.54 (m, 2858H), 3.38 (s, 93H), 3.23-3.09 (m, 104H), 2.50-2.48 (m, 64H), 1.90-1.32 (m, 378H).

### Dendrimer-Drug Conjugates

Schemes for the synthesis of example conjugates are shown in FIG. 1.

BHALys[Lys]₂[Lys]₄[Lys]₈[Lys]₁₆[Lys]₃₂[(α-NH-3′O-Glu-Remdesivir)₃₂(ε-NH-COPEG₂₀₀₀)₃₂] RHa-3

To a stirred solution of 3′ O-Glu-Remdesivir RHa-2 (80 mg, 0.104 mmol) and PyBOP (55 mg, 0.104 mmol) in DMF (1.0 mL) at rt was added a premixed solution of BHALys [Lys]₂[Lys]₄[Lys]₈[Lys]₁₆[Lys]₃₂[(α-NH₂)₃₂(ε-NH-COPEG₂₀₀₀)₃₂].32TFA RHa-6 (183 mg, 2.0 μmop and NMM (44 μL, 0.40 mmol) in DMF (3.0 mL). After stirring at rt for 18 h, the reaction mixture was diluted with MeCN (5 mL) and concentrated in vacuo. The residue was dissolved in the minimum amount of MeCN (˜1 mL) and purified by SEC [mobile phase, MeCN, fractions analysed by TLC (visualization by UV or 5% aq. BaCl₂ followed by staining with a solution of 12 in EtOH) and HPLC (HPLC Method 1)]. Fractions containing the product were concentrated in vacuo, dissolved in the minimum amount of water (˜2 mL), filtered (0.45 μm porosity syringe filter disc) and lyophilised to give BHALys[Lys]₂[Lys]₄[Lys]₈[Lys]₁₆[Lys]₃₂[(α-NH-3′ O-Glu-Remdesivir)₃₂(ε-NH-COPEG₂₀₀₀)₃₂] RHa-3 as a pale yellow solid (211 mg). ¹H NMR (300 MHz, CD₃OD): δ (ppm) 1.00-0.69 (m, 202H), 2.17-1.00 (m, 686H), 2.79-2.17 (m, 145H), 3.27-2.79 (m, 140H), 3.79-3.38 (m, 5319H), 3.35H (s, 96H), 4.72-3.79 (m, 360H), 5.24-5.02 (m, 27H), 5.50-5.24 (m, 28H), 5.50-5.24 (m, 28H), 6.00-5.81 (m, 6H) 7.41-6.70 (m, 2H) and 8.23-7.66 (m, 107H); HPLC (HPLC Method 1): Rₜ=8.97 min. Drug loading was assessed by ¹H NMR spectroscopy using 3,4,5-trichloropyridine as an internal standard, which showed a loading of 21.8% w/w Remdesivir RHa-1.

BHALys[Lys]₂[Lys]₄[Lys]₈[Lys]₁₆[Lys]₃₂[(α-NH-6N-TDA-Remdesivir)₃₂(ε-NH-COPEG₂₀₀₀)₃₂] RHa-5

To a stirred solution of 6N-TDA-Remdesivir RHa-4 (300 mg, 60% potency, 0.25 mmol) and PyBOP (130 mg, 0.25 mmol) in DMF (4.0 mL) at rt was added a premixed solution of BHALys[Lys]₂[Lys]₄[Lys]₈[Lys]₁₆[Lys]₃₂[(α-NH₂)₃₂(ε-NH-COPEG₂₀₀₀)₃₂].32TFA RHa-6 (431 mg, 5.8 μmol) and NMM (110 μL, 0.93 mmol) in DMF (6.0 mL). After stirring at rt for 18 h, the reaction mixture was concentrated in vacuo. The residue was dissolved in the minimum amount of MeCN (˜1 mL) and purified by SEC [mobile phase, MeCN, fractions analysed by TLC (visualization by UV or 5% aq. BaCl₂ followed by staining with a solution of 12 in EtOH)]. Fractions containing the product were concentrated in vacuo, dissolved in the minimum amount of water (˜5 mL), filtered (0.45 μm porosity syringe filter disc) and lyophilised to give BHALys [Lys]₂[Lys]₄[Lys]₈[Lys]₁₆[Lys]₃₂[(α-NH-6N-TDA-Remdesivir)₃₂(ε-NH-COPEG₂₀₀₀)₃₂] RHa-5 as a yellow solid (270 mg). ¹H NMR (300 MHz, MeOD): δ (ppm) 0.68-0.94 (m, 160H), 0.95-2.20 (m, 520H), 2.75-3.26 (m, 170H), 3.35 (s, 96H), 3.70-3.72 (m, 4896H), 3.72-4.63 (m, 411H), 6.95-7.45 (m, 180H), 7.60-8.64 (111H); HPLC (HPLC Method 2): Rₜ=7.93 min. Drug loading was assessed by ¹H NMR spectroscopy using 3,4,5-trichloropyridine as an internal standard, which showed a loading of 18.6% w/w Remdesivir RHa-1.

BHALys[Lys]₂[Lys]₄[Lys]₈[Lys]₁₆[Lys]₃₂[(α-NH-3′O-DGA-Remdesivir)₃₂(ε-NH-COPEG₂₀₀₀)₃₂] RHa-11

To a stirred solution of 3′O-DGA-Remdesivir RHa-10 (85 mg, 70% potency, 0.077 mmol) and PyBOP (41 mg, 0.077 mmol) in DMF (1.0 mL) at rt was added a solution of BHALys [Lys]₂[Lys]₄[Lys]₈[Lys]₁₆[Lys]₃₂[(α-NH₂)₃₂(ε-NH-COPEG₂₀₀₀)₃₂].32TFA RHa-6 (130 mg, 2.0 μmol) and NMM (33 μL, 0.29 mmol) in DMF (3 mL). The reaction mixture was stirred for 18 h and concentrated in vacuo. The residue was dissolved in MeCN (1.5 mL) and purified by SEC [mobile phase, MeCN, fractions analysed by TLC (visualization by UV or 5% aq. BaCl₂ followed by staining with a solution of 12 in EtOH)]. Fractions containing the product were concentrated in vacuo, dissolved in the minimum amount of water (˜5 mL), filtered (0.45 μm porosity syringe filter disc) and lyophilised to give BHALys[Lys]₂[Lys]₄[Lys]₈[Lys]₁₆[Lys]₃₂[(α-NH-3′ O-DGA-Remdesivir)₃₂(ε-NH-COPEG₂₀₀₀)₃₂] RHa-11 as a pale yellow solid (160 mg). ¹H NMR (300 MHz, MeOD): δ (ppm) 0.68-1.01 (m, 192H), 1.01-2.13 (m, 569H), 2.63-3.26 (m, 230H), 3.36 (s, 96H), 3.37-3.46 (m, 59H), 3.45-3.80 (m, 4941H), 3.80-4.75 (m, 494H), 5.02-5.35 (m, 27H), 5.35-5.62 (m, 25H), 5.94-6.05 (m, 3H), 6.67-7.06 (m, 60H), 7.06-7.51 (m, 160H), 7.51-8.19 (m, 88H). HPLC (HPLC Method 2): Rₜ=8.02 min. Drug loading was assessed by ¹H NMR spectroscopy using 3,4,5-trichloropyridine as an internal standard, which showed a loading of 17.7% w/w Remdesivir RHa-1.

BHALys[Lys]₂[Lys]₄[Lys]₈[Lys]₁₆[Lys]₃₂[(α-NH-3′O-TDA-Remdesivir)₃₂(ε-NH-COPEG₂₀₀₀)₃₂] RHa-15

To a stirred solution of 3′O-TDA-Remdesivir RHa-14 (110 mg, 94% potency, 0.135 mmol) and PyBOP (72 mg, 0.135 mmol) in DMF (1.0 mL) at rt was added a solution of BHALys [Lys]₂[Lys]₄[Lys]₈[Lys]₁₆[Lys]₃₂[(α-NH₂)₃₂(ε-NH-COPEG₂₀₀₀)₃₂].32TFA RHa-6 (237 mg, 3.0 mol) and NMM (57 μL, 0.52 mmol) in DMF (3 mL). The reaction mixture was stirred for 18 h and concentrated in vacuo. The residue was dissolved in MeCN (1.5 mL) and purified by SEC [mobile phase, MeCN, fractions analysed by TLC (visualization by UV or 5% aq. BaCl₂ followed by staining with a solution of 12 in EtOH)]. Fractions containing the product were concentrated in vacuo, dissolved in the minimum amount of water (˜5 mL), filtered (0.45 μm porosity syringe filter disc) and lyophilised to give BHALys [Lys]₂[Lys]₄[Lys]₈[Lys]₁₆[Lys]₃₂[(α-NH-3′ O-TDA-Remdesivir)₃₂(ε-NH-COPEG₂₀₀₀)₃₂] RHa-15 as a pale yellow solid (146 mg and 148 mg in two batches). ¹H NMR (300 MHz, MeOD): δ (ppm) 0.67-0.96 (m, 207H), 0.96-2.04 (m, 595H), 2.74-3.27 (m, 121H), 3.35 (s, 96H), 3.37-4.15 (m, 5399H), 4.18-4.73 (m, 161H), 5.06-5.56 (m, 59H), 5.89-6.01 (m, 5H), 6.70-7.06 (m, 66H), 7.06-7.51 (m, 179H), 7.60-8.39 (m, 99H). HPLC (HPLC Method 1): Rₜ=8.97 min. Drug loading was assessed by ¹H NMR spectroscopy using 3,4,5-trichloropyridine as an internal standard, which showed a loading of 19.7% w/w Remdesivir RHa-1.

BHALys[Lys]₂[Lys]₄[Lys]₈[Lys]₁₆[Lys]₃₂[(α-NH-6N-TDA-Remdesivir)₃₂(ε-NH-COPEG₁₀₀₀)₃₂] RHa-17

To a stirred solution of 6N-TDA-Remdesivir RHa-4 (400 mg, 70% potency, 0.38 mmol) in DMF (4.0 mL) was added PyBOP (520 mg, 0.38 mmol), BHALys [Lys]₂[Lys]₄[Lys]₈[Lys]₁₆[Lys]₃₂[(α-NH₂)₃₂(ε-NH-COPEG₁₀₀₀)₃₂].32TFA RHa-16 (500 mg, 0.01 mmol) followed by NMM (160 mg, 1.59 mmol). After stirring at rt for 18 h, the reaction mixture was diluted with MeCN (4.0 mL) and purified by TFF in MeCN (10 kDa MWCO, 0.11 m² Millipore Pellicon membrane, 40 DV). The purified solution was concentrated in vacuo, dissolved in water (10 mL), filtered (0.45 μm porosity syringe filter disc) and lyophilised to give BHALys[Lys]₂[Lys]₄[Lys]₈[Lys])₆[Lys]₃₂[(α-NH-6N-TDA-Remdesivir)₃₂(ε-NH-COPEG₁₀₀₀)₃₂] RHa-17 as an off-white solid (561 mg). ¹H NMR (300 MHz, MeOD) δ (ppm) 0.70-0.95 (m, 192H), 0.95-2.20 (m, 712H), 2.69-3.28 (m, 179H), 3.36 (s, 140H), 3.38-3.78 (m, 4113H), 3.78-4.62 (m, 428H), 6.16 (s, 1H), 6.97-7.50 (m, 219H), 7.65-8.15 (m, 56H), 8.25-8.42 (m, 29H); HPLC (HPLC Method 2): Rₜ=8.87 min. Drug loading was assessed by 11-1 NMR spectroscopy using 3,4,5-trichloropyridine as an internal standard, which showed a loading of 23.0% w/w Remdesivir RHa-1.

The % w/w drug moiety (Remdesivir) and calculated number of drug moieties per conjugate is shown below.

| | % Loading | Number of |
|---|---|---|
| Construct | Remdesivir | Remdesivir |
| RHa-3 | 21.8 | 32 |
| | | (Calc. = 33.7) |
| RHa-5 | 18.6 | 28.8 |
| RHa-11 | 17.7 | 27.4 |
| RHa-15 | 19.7 | 30.5 |
| RHa-17 | 23 | 26.7 |

BHALys[Lys]₂[Lys]₄[Lys]₈[Lys]₁₆[Lys]₃₂[(α-NH-5′O-DGA-GS441524)₃₂(ε-NH-COPEG₂₀₀₀)₃₂] RL-7

To a stirred solution of 5′O-DGA-GS441524 RL-4 (118 mg, 0.29 mmol) and BHALys [Lys]₂[Lys]₄[Lys]₈[Lys]₁₆[Lys]₃₂[(α-NH₂)₃₂(ε-NH-COPEG₂₀₀₀)₃₂].32TFA (496 mg, 6.7 μmol) in DMF (5 mL) was added and PyBOP (150 mg, 0.29 mmol) and DIPEA (116 μL, 0.67 mmol). After stirring at rt for 2 d, the reaction mixture was concentrated in vacuo. The residue was dissolved in the minimum amount of MeCN (˜1 mL) and purified by SEC [mobile phase, MeCN, fractions analysed by TLC (visualization by UV or 5% aq. BaCl₂ followed by staining with a solution of 12 in EtOH) and HPLC (HPLC Method 4)]. Fractions containing the product were concentrated in vacuo, dissolved in the minimum amount of water (˜2 mL), filtered (0.45 μm porosity syringe filter disc) and lyophilised to give BHALys [Lys]₂[Lys]₄[Lys]₈[Lys]₁₆[Lys]₃₂[(α-NH-5′ O-DGA-GS-441524)₃₂(ε-NH-COPEG₂₀₀₀)₃₂] RL-7 as an off white solid (500 mg). HPLC (HPLC Method 4): Rₜ=4.94 min.

BHALys[Lys]₂[Lys]₄[Lys]₈[Lys]₁₆[Lys]₃₂[(α-NH-5′O-TDA-GS441524)₃₂(ε-NH-COPEG₂₀₀₀)₃₂] RL-8

To a stirred solution of 5′O-TDA-GS441524 RL-6 (40 mg, 0.095 mmol) and BHALys [Lys]₂[Lys]₄[Lys]₈[Lys]₁₆[Lys]₃₂[(α-NH₂)₃₂(ε-NH-COPEG₂₀₀₀)₃₂].32TFA (189 mg, 2.5 μmop in DMF (5 mL) was added and PyBOP (50 mg, 0.0.095 mmol) and DIPEA (41 μL, 0.23 mmol). After stirring at rt for 18 h, the reaction mixture was concentrated in vacuo. The residue was dissolved in the minimum amount of MeCN (˜1 mL) and purified by SEC [mobile phase, MeCN, fractions analysed by TLC (visualization by UV or 5% aq. BaCl₂ followed by staining with a solution of 12 in EtOH) and HPLC (HPLC Method 4)]. Fractions containing the product were concentrated in vacuo, dissolved in the minimum amount of water (˜2 mL), filtered (0.45 μm porosity syringe filter disc) and lyophilised to give BHALys [Lys]₂[Lys]₄[Lys]₈[Lys]₁₆[Lys]₃₂[(α-NH-5′ O-TDA-GS-441524)₃₂(ε-NH-COPEG₂₀₀₀)₃₂] RL-8 as an off-white solid (80 mg). HPLC (HPLC Method 4): Rₜ=4.93 min.

### Example 2: Solubility Studies

### Solubility of RHa-3

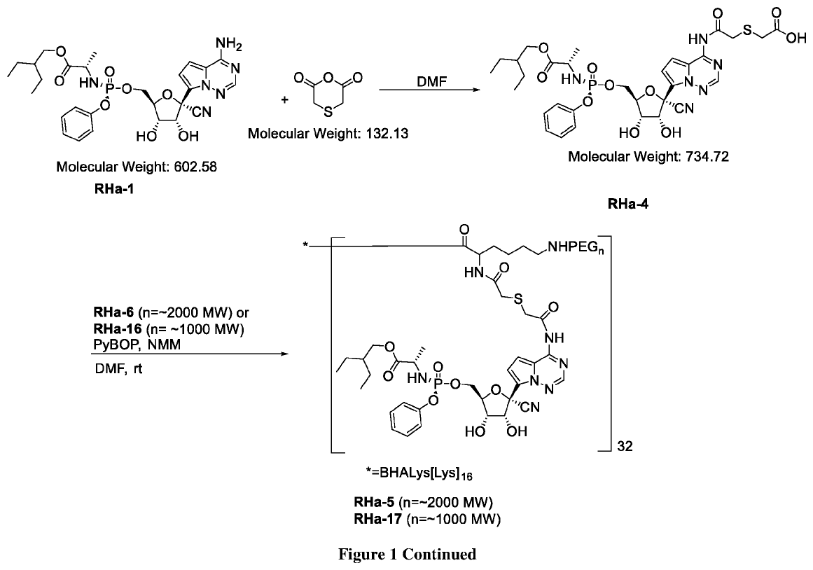

The solubility of BHALys[Lys]₂[Lys]₄[Lys]₈[Lys]₁₆[Lys]₃₂[(α-NH-3′ O-Glu-Remdesivir)₃₂(ε-NH-COPEG₂₀₀₀)₃₂] RHa-3 in water was determined to be ≥200 mg/mL (or ≥43.6 mg/mL Remdesivir RHa-1) by adding water (250 μL) to 50.0 mg of BHALys [Lys]₂[Lys]₄[Lys]₈[Lys]₁₆[Lys]₃₂[(α-NH-3′ O-Glu-Remdesivir)₃₂(ε-NH-COPEG₂₀₀₀)₃₂] and swirling briefly. After 2 min, the resulting solution was visually inspected for particulates and found to be a clear solution (see FIG. 2). Solutions shown are 40 mg/ml Remdesivir equivalents (equivalent to 200 mg/ml of conjugate RHa-3).

### Solubility of RHa-5

The solubility of BHALys[Lys]₂[Lys]₄[Lys]₈[Lys]₁₆[Lys]₃₂[(α-NH-6N-TDA-Remdesivir)₃₂(ε-NH-COPEG₂₀₀₀)₃₂] RHa-5, in EtOH:Water (1:9 v/v) was determined to be ≥543 mg/mL (or ≥107 mg/mL Remdesivir RHa-1) by adding water EtOH:Water (1:9 v/v, 400 μL) to 380 mg of RHa-5. The mixture was swirled briefly and left to stand at rt. After 1.5 h the resulting solution (volume recorded=700 μL) was visually inspected for particulates and found to be a clear solution.

### Solubility of RHa-17

The solubility of BHALys[Lys]₂[Lys]₄[Lys]₈[Lys]₁₆[Lys]₃₂[(α-NH-6N-TDA-Remdesivir)₃₂(ε-NH-COPEG₁₀₀₀)₃₂] RHa-17 in EtOH:Water (1:9 v/v) was determined to be ≥354* mg/mL (or ≥85 mg/mL Remdesivir RHa-1) by adding EtOH:Water (1:9 v/v, 958 μL) portionwise to 525 mg of RHa-17, over a period of 1.5 h with intermittent swirling. The resulting solution was visually inspected for particulates and found to be a clear solution.

(*Total volume not recorded. Assumes 1 mg construct contributes to 1 μL of final solution (i.e., total volume of final solution=1483 μL)).

### Example 3: Release of Remdesivir (RDV)

The extent to which the Remdesivir RHa-1 was released over time in PBS buffer (with 10% v/v DMSO) (pH 7.4) at 37° C. was determined for 5 constructs, RHa-17, RHa-15, RHa-11, RHa-5 and RHa-3 (Release Method 1) over 120 hours.

Trace amounts of other components were also observed (presumed to be degradation products of Remdesivir RHa-1). The area under the resolvable (separated) peaks associated with these degradants was also added to the peak area associated with released Remdesivir RHa-1.

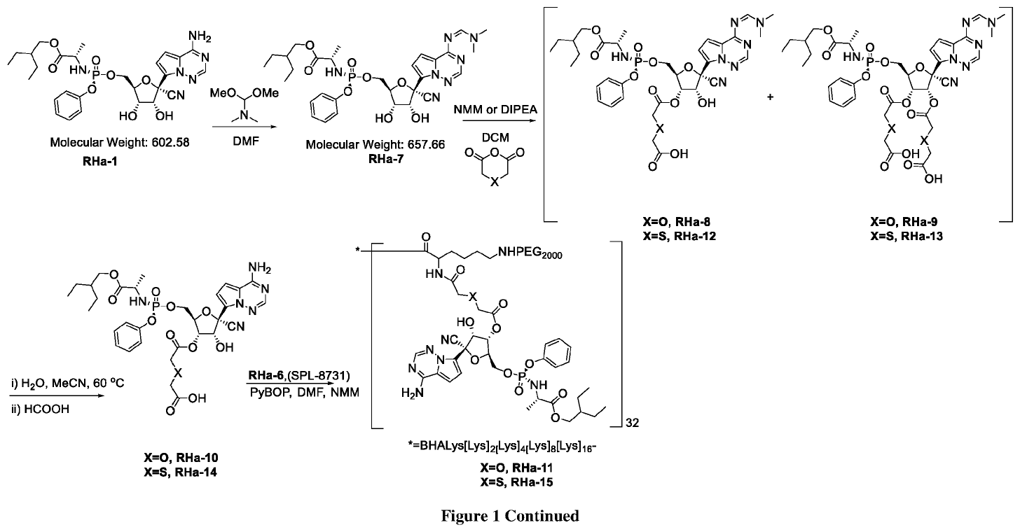

BHALys [Lys]₂[Lys]₄[Lys]₈[Lys]₁₆[Lys]₃₂[(α-NH-6N-TDA-Remdesivir)₃₂(ε-NH-COPEG₂₀₀₀)₃₂] RHa-5 contained 1.77% free Remdesivir RHa-1 at t=0 min. The value of released Remdesivir RHa-1 shown in FIG. 3 has been adjusted to take this into account.

TABLE 4
Time to 50% release (hrs)

| | | Approx. Time taken for |
|---|---|---|
| Linker Type | Number | 50% Remdesivir release |
| O-DGA | RHa-11 | 3 hrs |
| O-TDA | RHa-15 | 9 hrs |
| N-TDA PEG₁₀₀₀ | RHa-17 | 48 hrs |
| N-TDA PEG₂₀₀₀ | RHa-5 | 58 hrs |
| O-Glu | RHa-3 | >120 hrs |

O-DGA and O-TDA linkers provided fast release rate of less than 10 hours to achieve 50% release of drug. O-Glu linker provided very slow release of greater than 120 hours to achieve 50% release of the drug. N-TDA linker provided medium release rate of about 50 hours to achieve 50% release of the drug. The linkers described enable controlled of release of the drug moiety.

### Example 4: Viscosity Measurements

Viscosities of 2 formulations containing either RHa-5 or RHa-17 in EtOH: Water (1:9 v/v) were measured using Viscosity Method 1

TABLE 5
Viscosities at 50 mg/mL Remdesivir equivalents in EtOH: Water (1:9 v/v)

| | Viscosity at 20° C. | Viscosity at 25° C. |
|---|---|---|
| Dendrimer | (speed, Torque) | (speed, Torque) |
| BHALys[Lys]₂[Lys]₄[Lys]₈[Lys]₁₆[Lys]₃₂[(α- | 38.99 cP | 29.32 cP |
| NH-6N-TDA-Remdesivir)₃₂(ε-NH- | (63 rpm, 79.9%) | (90 rpm, 79.9%) |
| COPEG₁₀₀₀)₃₂] RHa-17 | | |
| BHALys[Lys]₂[Lys]₄[Lys]₈[Lys]₁₆[Lys]₃₂[(α- | 144.2 cP | 100.6 cP |
| NH-6N-TDA-Remdesivir)₃₂(ε-NH- | (100 rpm, 34.3%) | (100 rpm, 25.8%) |
| COPEG₂₀₀₀)₃₂] RHa-5 | | |

### Example 5: Pharmacokinetics of Dendrimer-Doxorubicin or Doxorubicin after Pulmonary Instillation to Rats

The ability to deliver dendrimer-drug conjugates via the pulmonary route was evaluated. A dendrimer-doxorubicin conjugate (D-DOX) (Kaminskas L M, et al, Nanomedicine. 2012 January; 8(1):103-11) was freeze dried and stored at −20° C. until required. D-DOX was reconstituted in pH 7.4 PBS to 30 mg/ml (4.5 mg/ml doxorubicin equivalents) immediately prior to dosing. The drug-free dendrimer control (D) contained PEG1100 and the 4-(hydrazinosulphonyl) benzoic acid linker on the surface, but without doxorubicin as has been described previously (Kaminskas L M et al, J Control Release. 2011 Jun. 10; 152(2):241).

Male Sprague Dawley (SD) rats (8-9 weeks, 270-320 g) were obtained from Monash Animal Services (VIC, Australia). Female F344 rats (8-10 weeks) were supplied by Animal Resources Centre (Perth, Australia).

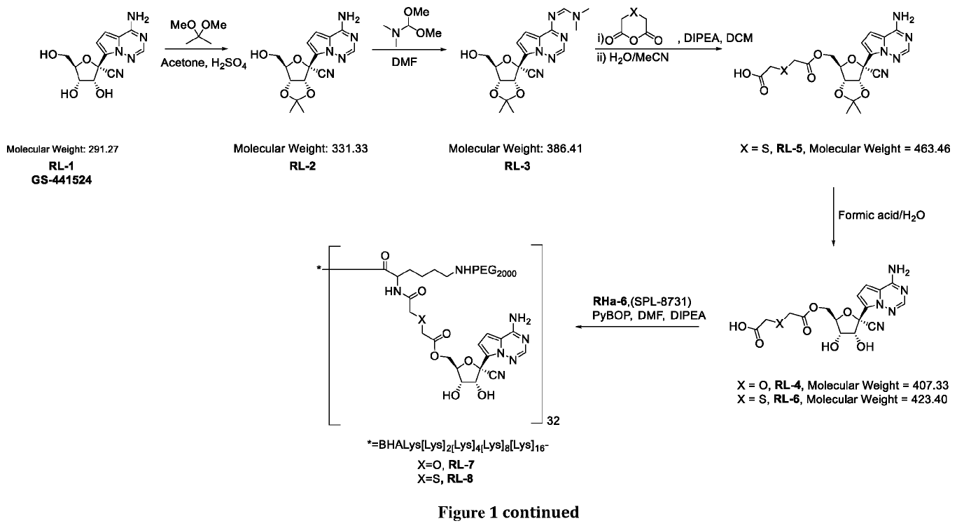

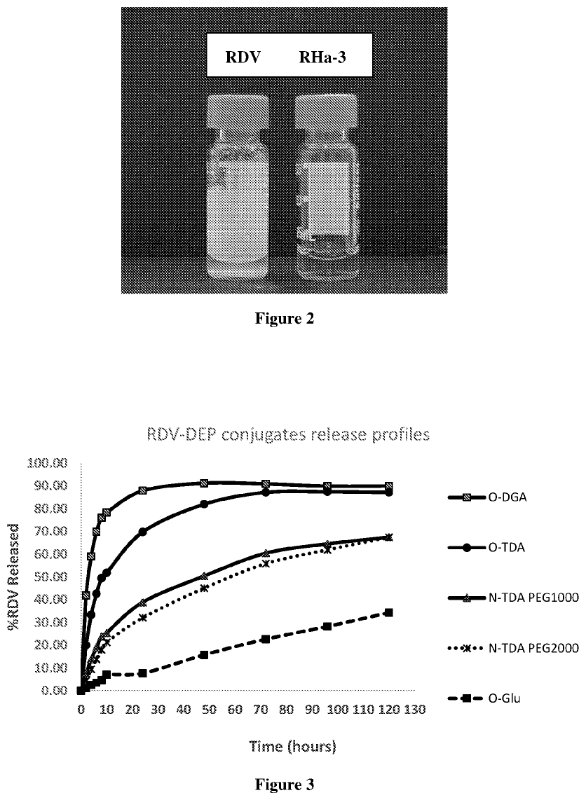

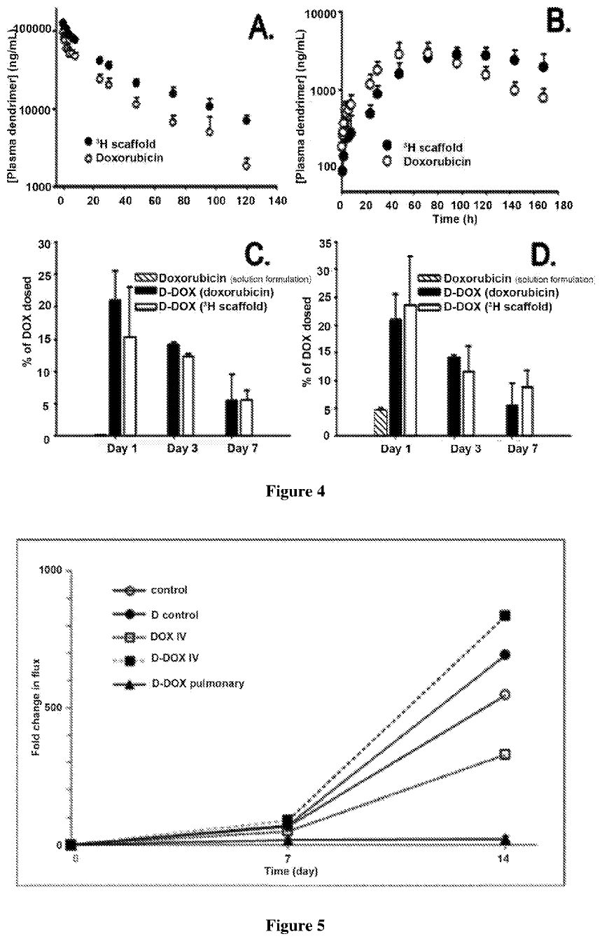

SD rats were cannulated via the right carotid artery and jugular vein for collection of blood samples and IV dosing respectively Rats were intravenously administered 1 ml of doxorubicin or D-DOX in sterile saline over 2 mins to provide a final dose of −0.75 mg/kg doxorubicin equivalents. Blood samples and urine were then collected for up to 5 days. For the pharmacokinetic evaluation of doxorubicin or D-DOX administered to the lungs as a liquid instillation, rats were cannulated only via the right carotid artery. Rats were administered 0.6 mg of doxorubicin or 1 mg of D-DOX in 100 μl saline via intratracheal instillation to the lungs of rats under isoflurane anaesthesia. Blood was sampled from doxorubicin dosed rats for 24 h and from D-DOX dosed rats for 7 days and urine and feces were collected over the sampling period. At the completion of the study, bronchoalveolar lavage fluid (BALF), lungs, liver, heart, spleen, kidneys and pancreas were collected from rats for biodistribution analysis. BALF was collected via the intratracheal perfusion of 3×5 ml saline Alveolar macrophages were isolated from BALF via centrifugation at 600×g for 10 mins. In addition, in a separate cohort of rats administered D-DOX via intratracheal instillation, BALF and lungs were collected 1 or 3 days after dosing to obtain lung biodistribution data over time. Plasma, urine, feces, and organs were analysed for radiolabel via liquid scintillation counting. Total doxorubicin in plasma, BALF and lung tissue in pulmonary dosed rats was analysed via a fluorescence HPLC (Kaminskas L M, et al, Nanomedicine. 2012 January; 8(1):103-11). Results are shown in FIGS. 4 to 6. The results show residency in the lung and clearance of the dendrimer-drug conjugate from the lungs over time.

Pharmacokinetic parameters for D-DOX have been normalized to 5 mg/kg for both the ³H-scaffold and doxorubicin. Pharmacokinetic parameters for doxorubicin have been normalized to 0.75 mg/kg (reflecting the dose of doxorubicin given at a dose of 5 mg/kg D-DOX). Pharmacokinetic parameters for intravenously administered D-DOX (by following the ³H-labelled scaffold) were reported previously (Kaminskas L M et al, 2012). Data are represented as mean±s.d. (n=3-7).

| | | Dendrimer-doxorubicin (D-DOX) | | |
|---|---|---|---|---|
| | Units | ³H-scaffold | Doxorubicin | Doxorubicin |
| | | INTRAVENOUS ADMINISTRATION | | |
| AUC | μg/ml · h | 4684 ± 939 | 1980 ± 340 | 0.21 ± 0.04 |
| Kₑₗ | ⁻h | 0.014 ± 0.002 | 0.028 ± 0.005 | 0.518 ± 0.312 |
| Excretion | % | 13 ± 5 | ND | ND |
| in urine | | | | |
| | | INTRATRACHEAL INSTILLATION | | |
| AUC | μg/ml · h | 550 ± 159 | 350 ± 107 | 0.17 ± 0.02 |
| Kₑₗ | ⁻h | 0.008 ± 0.003 | 0.015 ± 0.003 | 0.132 ± 0.010 |
| t_{1/2} | H | 96 ± 41 | 49 ± 11 | 5 ± 0.4 |
| Cₘₐₓ | ng/ml | 3232 ± 691 | 3011 ± 1095 | 125 ± 16 |
| Tₘₐₓ | H | 106 ± 23 | 66 ± 23 | 0.1 ± 0.05 |
| F_{abs} 0-7 d | % | 7 ± 2 | 15 ± 5 | ND |
| F_{abs} 0-∞ | % | 12 ± 3 | 18 ± 6 | 83 ± 9 |
| Excretion | % | 7 ± 1 | ND | ND |
| in urine | | | | |

After intratracheal instillation of the dendrimer, between 12-18% of the dendrimer and dendrimer-associated doxorubicin access the systemic circulation (calculated to infinity). The majority of the D-DOX being retained within the lung at the measured time.

### Example 6: Localization of PEGylated Dendrimers in the Lungs after Intratracheal Instillation

A generation 5 polylysine dendrimer bearing PEG˜1100 conjugated to α-amino groups on the surface and dansyl fluorophore conjugated stably via an amide linkage to surface ε-amino groups (prepared by analogous synthetic procedures to those described in Example 1) was administered via intratracheal instillation to the lungs of 2 rats (1 mg in 100 μl saline). Rats were euthanized after 30 mins or 2 days and the lungs removed and imaged on a Caliper in vivo imaging system to identify the location of the dendrimer dose within the lungs at the various times.

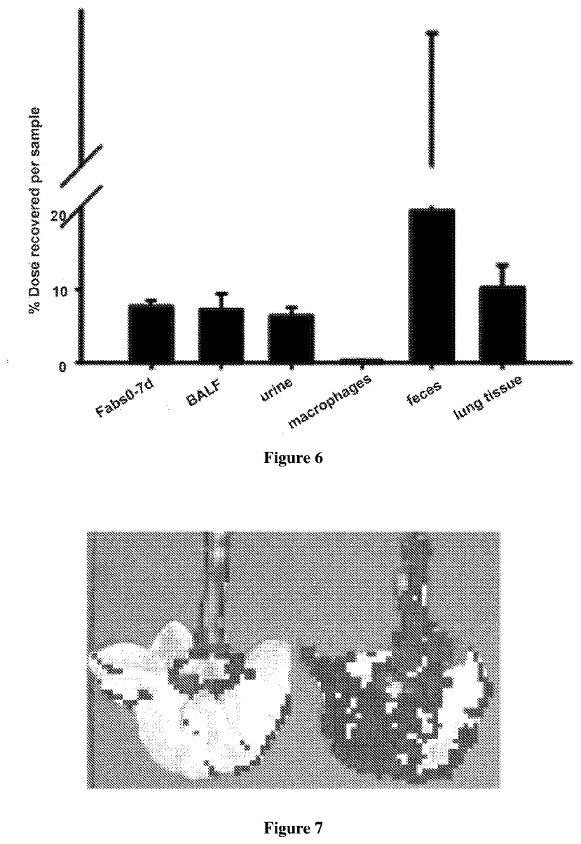

The results showed that dendrimer was distributed throughout the lung over time. Representative images are shown in FIG. 7.

### Example 7: Biodegradability of Dendrimers after Intratracheal Delivery. Size Exclusion Chromatographic Identification of Radiolabelled Species

To understand the fate of IT administered dendrimer, the distribution of dendrimer in lung tissue, BALF, alveolar macrophages, urine and faeces was determined via scintillation counting at various time points following IT dosing in male Sprague dawley rats (n=2). Generation 4 polylysine dendrimers were conjugated at surface amino groups with PEG₂₀₀, PEG₅₇₀ or PEG₂₃₀₀ to give a range of constructs with varying molecular weights (11-78 kDa). With the aid of a laryngoscope, the polyethylene cannula was inserted into the trachea to a distance of 2.5 cm past the larynx. The dendrimer solution was instilled as a bolus into the lungs in time with inhalation and once the cannula was withdrawn from the lungs a 150 μl t0 blood sample was collected. Blood samples taken after t0 were collected at 2, 8 and 24 h after intratracheal instillation of dendrimer. After the last blood sample was collected, rats were sacrificed under isoflurane anaesthesia by exsanguination from the carotid artery cannula. Lungs were excised from rats and stored at −20° C. until processing.

Lung tissue homogenate supernatant was prepared as previously described. Samples of plasma, urine, BALF and lung homogenate supernatant were analysed via size exclusion chromatography on a Superdex 75 column. Samples (100-200 μl) were injected onto the column which was eluted with 50 mM phosphate buffer (pH 3.5) containing 0.3 M NaCl as previously described Boyd B J, Kaminskas L M, Karellas P, Krippner G, Lessene R, Porter C J H. Cationic Poly-1-lysine Dendrimers: Pharmacokinetics, Biodistribution, and Evidence for Metabolism and Bioresorption after Intravenous Administration to Rats. Molecular Pharmaceutics. 2006; 3 (5):614-27).

Column eluate was collected at 1 min intervals over 50 mins into 6 ml scintillation vials, mixed with IRGASafe scintillation cocktail and analysed via liquid scintillation counting. To determine the proportion of total radioactivity in each SEC sample that was present as intact dendrimer or lower or higher molecular weight products, the percentage of the total area under the SEC curve (AUC, calculated using the trapezoidal method) for the peak of interest was compared with the total AUC for all peaks present.

Previous data has shown that IV delivery results in moderate levels of breakdown products in the urine at 7 days. Despite low systemic absorption of the dendrimer scaffold following IT delivery, relatively high levels of dendrimer breakdown products are observed in the urine at the 7-day time point. This indicates that the dendrimer is degraded in the lung following IT delivery. Biodegradability is important to ensure no long-term toxicities result from accumulation of the dendrimer vehicle in the lung.

### Example 8: Pharmacokinetics of BHALys[Lys]₂[Lys]₄[Lys]₈[Lys]₁₆(PEG₂₀₀₀)₃₂ Dendrimer after Aerosol Administration to the Lungs of Rats with a Penn Century Microsprayer

The dendrimer BHALys[Lys]₂[Lys]₄[Lys]₈[Lys]₁₆(PEG₂₀₀₀)₃₂ was constituted to 10 mg/ml in PBS and stored at −20° C. until required. The construction of the dendrimer was as described previously (Kaminskas L M, et al Mol Pharm. 2008 May-June; 5(3):449-63).

Male Sprague Dawley (SD) rats (8-9 weeks, 270-320 g) were obtained from Monash Animal Services (VIC, Australia). SD rats were cannulated via the right carotid artery and jugular vein for collection of blood samples and IV dosing respectively Rats were dosed IV with 5 mg/kg dendrimer and via the lungs with 1 mg dendrimer. Rats were intravenously administered 1 ml of dendrimer in sterile saline over 2 mins. Blood samples and urine were then collected for up to 7 days. For the pharmacokinetic evaluation of dendrimer administered to the lungs as an aerosolized dose, rats were cannulated only via the right carotid artery. Rats were administered 1 mg of dendrimer in 100 μl saline via intratracheal insertion of the Penn Century micro spray aerosol device to the lungs of rats under isoflurane anaesthesia. Blood was sampled from dendrimer dosed rats for 7 days and urine and feces were collected over the sampling period. At the completion of the study, bronchoalveolar lavage fluid (BALF) and lungs were collected from rats for biodistribution analysis. BALF was collected via the intratracheal perfusion of 3×5 nil saline Alveolar macrophages were isolated from BALF via centrifugation at 600×g for 10 mins. Plasma, urine, feces and lungs were analysed for radiolabel via liquid scintillation counting

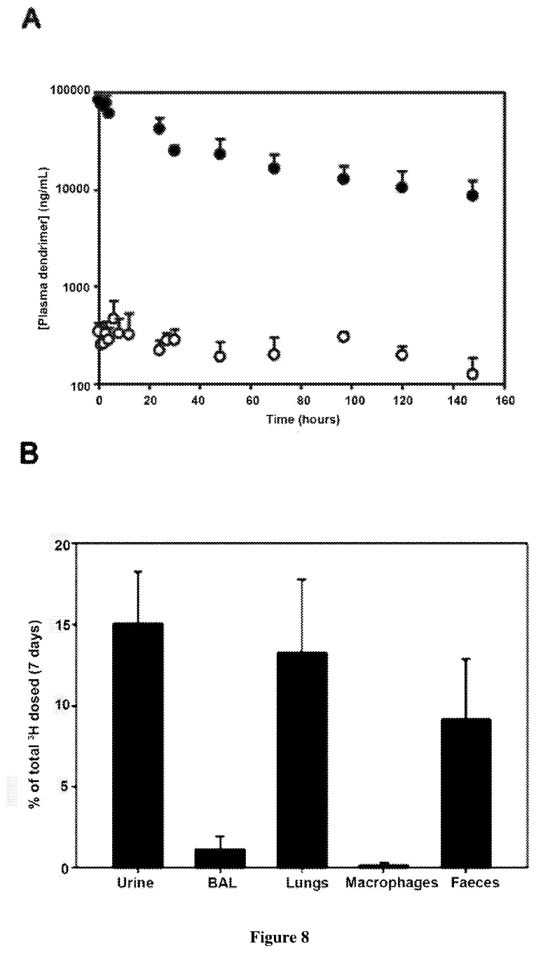

Results are shown in FIGS. 8A and B. Data reported in Figure A represents mean±s.d. (n=4-5 rats) and data is normalized to 5 mg/kg dendrimer. The bioavailability of the dendrimer after lung delivery was 2.4±3.3%. Data in Figure B represents mean±s.d. (n=4 rats) 7 days after the pulmonary dose. The data indicates that a large proportion of the large dendrimer is retained in the lungs, and not rapidly cleared.

### Example 9: Pharmacokinetic Studies of RHa-5 and RHa-15

Studies were undertaken to investigate the pharmacokinetic (PK) profile of two Remdesivir dendrimer conjugates following single intravenous (IV) or subcutaneous (SC) administration, and to compare them with that of Remdesivir (RDV). Analysis was conducted for both Remdesivir and its active metabolite GS-441524.

The study consisted of two phases (PK Phase 1 and PK Phase 2) in a total of 6 animals (age 36-48 months) each part separated by a washout period:
PK Phase 1: Four animals (mammals) were studied. One male and one female were administered RHa-5 (BHALys [Lys]₂[Lys]₂[Lys]₈[Lys]₁₆[Lys]₃₂[(α-NH-6N-TDA-Remdesivir)₃₂(ε-NH-COPEG₂₀₀₀)₃₂] 20% Remdesivir loading w/w) (test item) intravenously; and one male and one female were administered RHa-5 subcutaneously. Blood samples were taken over a period of 7 days.
   A minimum washout period of 14 days took place between the last blood sampling point of the PK Phase 1 and the start of PK Phase 2.
PK Phase 2: Six animals (mammals) were studied. One male and one female were administered RHa-15 (BHALys[Lys]₂[Lys]₄[Lys]₈[Lys]₁₆[Lys]₃₂[(α-NH-3′ O-TDA-Remdesivir)₃₂(ε-NH-COPEG₂₀₀₀)₃₂] 18.63% Remdesivir loading w/w) (test item) intravenously; one male and one female were administered RHa-15 subcutaneously; and one male and one female were administered RDV (reference item). Blood samples were taken over a period of 7 days.

### PK Phase I and Phase 2 Formulation and Administration

Formulation procedure: Test items RHa-5 and RHa-15 were reconstituted in Required volume of Water for injection (WFI), until a complete solution was obtained. The required volume of WFI was slowly added to RDV formulation (a lyophilate of 3.2% w/w RDV, 96.8% and sodium sulfobutylether-β-cyclodextrin adjusted to pH 3.5 with HCl) and shaken vigorously for 30 seconds. The solution was left for 20 minutes to ensure complete dissolution.

Administration: The clinical Remdesivir loading dose in humans is 200 mg. Assuming a 60 kg human, this results in a dose of 3.33 mg/kg. Allometric conversion of the dose from humans to small mammals results in an equivalent test dose of 6.17 mg/kg Remdesivir. Test item was administered as follows:

PK Phase 1: Two animal groups (A and B) of one male and one female each were administered test item RHa-5 at a Remdesivir equivalent dose of 6.17 mg/kg. Group A were administered the test item as a slow (30 seconds) intravenous bolus; and Group B were administered the test item over 30 seconds using the subcutaneous (SC) route into the back of the animal.

PK Phase 2: Three animal groups (C, D and E) of one male and one female each were included in this study phase. Groups C and D were administered test item RHa-15 at a Remdesivir equivalent dose of 6.17 mg/kg using the IV and SC routes, respectively. Group E were administered 6.17 mg/kg of RDV intravenously. IV administrations were done as a slow (30 seconds) bolus. SC administrations were done over 30 seconds.

TABLE
Reconstitution and administration volumes:

| | Amount | Qty API | | | | Reconstitution | | | |
|---|---|---|---|---|---|---|---|---|---|
| Test | material | in vial | Animal | Animal | Dose | volume | Dose | | |
| Article | (mg) | (mg) | # | weight | (mg) | (mL) | (mL) | ROA | Group |
| RHa-5 | 426.12 | 79.39 | 1M | 10.81 | 66.70 | 2.380 | 2 | IV | A |
| RHa-5 | 425.33 | 79.24 | 2M | 11.14 | 68.73 | 2.306 | 2 | SC | B |
| RHa-5 | 457.07 | 85.15 | 4F | 9.92 | 61.21 | 2.782 | 2 | IV | A |
| RHa-5 | 451.40 | 84.10 | 5F | 9.99 | 61.64 | 2.729 | 2 | SC | B |
| RHa-15 | 454.00 | 90.75 | 1M | 11.16 | 68.86 | 2.636 | 2 | IV | C |
| RHa-15 | 453.48 | 90.65 | 2M | 11.11 | 68.55 | 2.645 | 2 | SC | D |
| RHa-15 | 431.64 | 86.28 | 4F | 9.22 | 56.89 | 3.034 | 2 | IV | C |
| RHa-15 | 433.80 | 86.72 | 5F | 9.64 | 59.48 | 2.916 | 2 | SC | D |
| RDV | 2790.30 | 89.14 | 3M | 10.05 | 62.01 | 17.251 | 12 | IV | E |
| RDV | 2798.70 | 89.41 | 6F | 11.82 | 72.93 | 14.712 | 12 | IV | E |

Sampling schedule: Blood samples for PK analysis were collected as follows:
Day 1: pre-dose and at 15 min, 30 min, 1 h, 2 h, 4 h, 6 h, and 12 h after administration.
Days 2 to 8: 24, 48, 72, and 168 h after administration.

Blood collection time-points were established from completion of administration of the entire dose volume.

PK Sample Collecting and Processing:

A blood volume of 2 mL was collected at each time point from the jugular vein with a 20-21G needle into a lithium heparin tube, and transferred into a tube containing 200 μL 0.15 M citric acid. Immediately following blood collection, the blood samples were mixed gently and kept on crushed ice until centrifugation at 1500 G for 10 min at 4° C. within 30 minutes of collection. Each plasma sample was stored frozen (˜80±10° C.).

### Pharmacokinetic Analysis

The concentration of Remdesivir and GS-441524 was determined in plasma samples using research grade liquid chromatography-mass spectrometry methods (LC/MS). The quantification range was 0.6 to 14400 ng/mL for Remdesivir and 0.6 to 14400 ng/mL for G441524.

Non-compartmental pharmacokinetic analysis was adopted to obtain the pharmacokinetic parameters from the blood concentration profiles. All values reported as below the lower limit of quantitation (0.6 ng/mL for Remdesivir and GS-441524) were assumed to be zero for the calculations.

The maximum concentration, Cₘₐₓ, and time of maximum concentration, Tₘₐₓ, were determined as the maximum measured concentration and its associated time. The area under the plasma concentration curve from 0 hours to the time of the last measurable concentration, AUC₀₋ₗₐₛₜ, was calculated using linear trapezoidal estimation. The calculation of the PK parameters: half-life (t_{1/2}), area under the plasma concentration curve from 0 hours to infinity (AUC_{0-inf}), volume of distribution during terminal elimination (V_{z}) and total body clearance (CL); required at least three data points in the terminal elimination phase. All the PK parameters were obtained using Kinetica 5.0 software (Thermo Scientific, Waltham, USA).

### Results

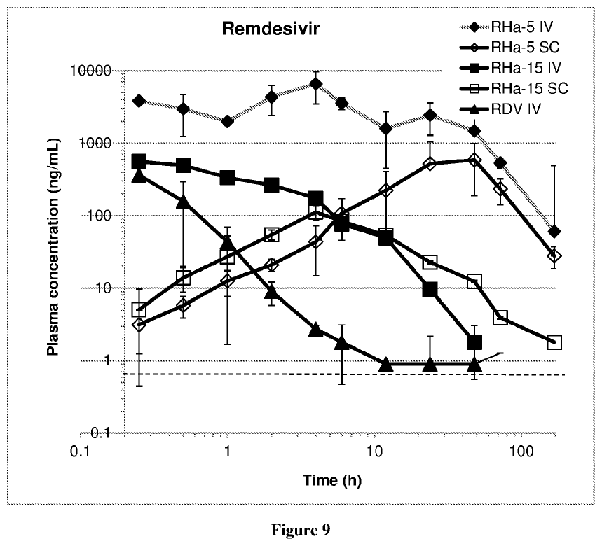

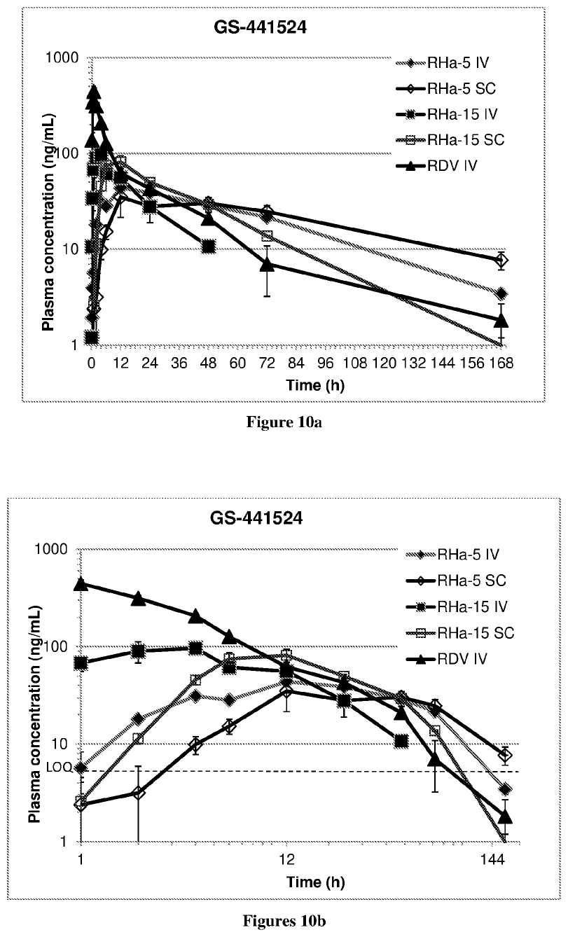

Remdesivir and GS-441524 were measured for samples taken. Results are shown in FIGS. 9 and 10. PK parameters were calculated as described above.

TABLE
PK parameters for Remdesivir and GS-441524

| | Remdesivir | | | | GS-441524 | | | |
|---|---|---|---|---|---|---|---|---|
| RHa-5 (IV) | 1M | 4F | Mean | SD | 1M | 4F | Mean | SD |
| Cₘₐₓ (ng/mL) | 7,061 | 6,553 | 6,807 | 359 | 39.5 | 51.0 | 45 | 8 |
| Tₘₐₓ (h) | 4 | 2 | 3.0 | 1.4 | 24 | 12 | 18.0 | 8.5 |
| C₀ (ng/mL) | 5701 | 4648 | 5175 | 744 | 2 | 2 | 2 | 0 |
| Cₗₐₛₜ (ng/mL) | 98.7 | 22.0 | 60 | 54 | 3.1 | 3.7 | 3 | 0 |
| tₗₐₛₜ (h)* | 168 | 168 | 168 | 0 | 168 | 168 | 168 | 0 |
| t_{1/2last} (h) | 29.5 | 19.2 | 24.3 | 7.3 | 36.6 | 39.6 | 38.1 | 2.1 |
| AUC₍₀₋ₗₐₛₜ₎ (ng*h/ml) | 157781 | 176696 | 167239 | 13375 | 3389 | 3507 | 3448 | 84 |
| AUC_{(0-inf)} (ng*h/ml) | 161978 | 177304 | 169641 | 10837 | 3552 | 3718 | 3635 | 117 |
| Vz (mL/kg) | 19438 | 11554 | 15496 | 5575.0 | | | | |
| CL (mL/h/kg) | 457 | 418 | 437 | 27.9 | | | | |
| F AUC₍₀₋ₗₐₛₜ₎ (x-fold) | 438.1 | 667.2 | 553 | 162 | 0.8 | 0.8 | 1 | 0 |
| F AUC_{(0-inf)} (x-fold) | 437.4 | 605.7 | 521.6 | 119.0 | 0.9 | 0.8 | 0.8 | 0.0 |

| | Remdesivir | | | | GS-441524 | | | |
|---|---|---|---|---|---|---|---|---|
| RHa-5 (SC) | 2M | 5F | Mean | SD | 2M | 5F | Mean | SD |
| Cₘₐₓ (ng/mL) | 897 | 306 | 602 | 418 | 45 | 27 | 36 | 13 |
| Tₘₐₓ (h) | 24.0 | 48.0 | 36.0 | 17 | 12.0 | 48.0 | 30.0 | |
| Cₗₐₛₜ (ng/mL) | 34.4 | 21.1 | 28 | 9 | 8.8 | 6.5 | 7.7 | 1.6 |
| tₗₐₛₜ (h)* | 168 | 168 | 168 | | 168 | 168 | 168 | |
| t_{1/2last} (h) | 28.9 | NC | 28.9 | — | 61.9 | NC | 61.9 | — |
| AUC₍₀₋ₗₐₛₜ₎ (ng*h/ml) | 52914 | 30133 | 41524 | 16108 | 3953 | 2978 | 3465 | 689 |
| AUC_{(0-inf)} (ng*h/ml) | 54347 | NC | 54347 | — | 4738 | NC | 4738 | — |
| V_{z} (mL/kg) | 56763 | NC | 56763 | | | | | |
| CL (mL/h/kg) | 1362 | NC | 1362.4 | | | | | |
| F AUC₍₀₋ₗₐₛₜ₎ (x-fold) | 146.9 | 113.8 | 130 | 23 | 1.0 | 0.7 | 1 | 0 |
| F AUC_{(0-inf)} (x-fold) | 146.8 | NC | 147 | | 1.1 | NC | 1.1 | |

| | Remdesivir | | | | GS-441524 | | | |
|---|---|---|---|---|---|---|---|---|
| RHa-15 (IV) | 1M | 4F | Ave | SD | 1M | 4F | Ave | SD |
| Cₘₐₓ (ng/mL) | 601 | 640 | 620 | 28 | 104.0 | 105.4 | 105 | 1 |
| Tₘₐₓ (h) | 0.25 | 0.25 | 0.3 | 0.0 | 6 | 4 | 5.0 | 1.4 |
| C₀ (ng/mL) | 664 | 691 | 677 | 19 | 1 | 1 | 1 | 0 |
| Cₗₐₛₜ (ng/mL) | 1.8 | 1.8 | 2 | 0 | 10.9 | 10.5 | 11 | 0 |
| tₗₐₛₜ (h)* | 72 | 72 | 2 | | 72 | 72 | 72 | |
| t_{1/2last} (h) | 10.0 | 10.1 | 10.0 | 0.0 | 21.5 | 19.0 | 20.3 | 1.8 |
| AUC₍₀₋ₗₐₛₜ₎ (ng*h/ml) | 4460 | 4310 | 4385 | 106 | 2901 | 3193 | 3047 | 206 |
| AUC_{(0-inf)} (ng*h/ml) | 4486 | 4336 | 4411 | 106 | 3240 | 3481 | 3360 | 170 |
| V_{z} (mL/kg) | 238470 | 248368 | 243419 | 6999 | | | | |
| CL (mL/h/kg) | 16505 | 17075 | 16790 | 403 | | | | |
| F AUC₍₀₋ₗₐₛₜ₎ (x-fold) | 12.4 | 16.3 | 14 | 3 | 0.7 | 0.7 | 1 | 0 |
| F AUC_{(0-inf)} (x-fold) | 12.1 | 14.8 | 13.5 | 1.9 | 0.8 | 0.8 | 0.8 | 0.0 |

| | Remdesivir | | | | GS-441524 | | | |
|---|---|---|---|---|---|---|---|---|
| RHa-15 (SC) | 2M | 5F | Mean | SD | 2M | 5F | Mean | SD |
| Cₘₐₓ (ng/mL) | 93 | 130 | 112 | 26 | 90 | 83 | 87 | 5 |
| Tₘₐₓ (h) | 4.0 | 4.0 | 4.0 | 0 | 12.0 | 6.0 | 9.0 | 4 |
| Cₗₐₛₜ (ng/mL) | 2 | 2 | 2 | 0 | 1 | 1 | 1 | 0 |
| tₗₐₛₜ (h)* | 168 | 168 | 168 | | 168 | 168 | 168 | |
| t_{1/2last} (h) | 41.3 | 31.5 | 36.4 | 7.0 | 26.2 | 24.5 | 25.3 | 1.2 |
| AUC₍₀₋ₗₐₛₜ₎ (ng*h/ml) | 2206 | 2148 | 2177 | 40 | 3565 | 3658 | 3612 | 66 |
| AUC_{(0-inf)} (ng*h/ml) | 2313 | 2230 | 2272 | 59 | 3607 | 3690 | 3648 | 59 |
| V_{z} (mL/kg) | 1909370 | 1506840 | 1708105 | 284632 | | | | |
| CL (mL/h/kg) | 32011 | 33199 | 32605 | 840 | | | | |
| F AUC₍₀₋ₗₐₛₜ₎ (x-fold) | 12.4 | 16.3 | 14 | 3 | 0.9 | 0.8 | 1 | 0 |
| F AUC_{(0-inf)} (x-fold) | 12.1 | 14.8 | 13.5 | 1.9 | 0.9 | 0.8 | 0.8 | 0.0 |

| | Remdesivir | | | | GS-441524 | | | |
|---|---|---|---|---|---|---|---|---|
| RDV (IV) | 3M | 6F | Mean | SD | 3M | 6F | Mean | SD |
| Cₘₐₓ (ng/mL) | 462 | 267 | 364 | 138 | 471.7 | 456.6 | 464 | 11 |
| Tₘₐₓ (h) | 0.25 | 0.25 | 0.3 | 0.0 | 1 | 0.5 | 0.8 | 0.4 |
| Clast (ng/mL) | 1.8 | 1.8 | 2 | 0 | 2.4 | 1 | 2 | 1 |
| tₗₐₛₜ (h)* | 24 | 48 | 36 | | 168 | 168 | 168 | |
| t_{1/2last} (h) | 3.9 | 10.7 | 7.3 | 4.8 | 29.6 | 27.6 | 28.6 | 1.4 |
| AUC₍₀₋ₗₐₛₜ₎ (ng*h/ml) | 360 | 265 | 312 | 67 | 4031 | 4470 | 4250 | 311 |
| AUC_{(0-inf)} (ng*h/ml) | 370 | 293 | 332 | 55 | 4133 | 4518 | 4326 | 272 |
| V_{z} (mL/kg) | 1133060 | 3915230 | 2524145 | 1967291 | | | | |
| CL (mL/h/kg) | 199938 | 252944 | 226441 | 37481 | | | | |

Both RHa-5 and RHa-15 delivered IV or SC show substantially extended plasma Remdesivir t_{1/2} and increased AUC, compared to RDV (reference product) delivered IV. As expected, RDV delivered IV was rapidly cleared from blood.

Dendrimer-drug conjugates provided plasma levels of Remdesivir greater than 10 ng/ml for at least 7 days. In comparison, RDV (unconjugated) provided plasma levels of greater than 10 ng/ml for about 1 hour. Similarly, dendrimer-drug conjugates provided plasma levels of Remdesivir greater than 100 ng/ml for at least 3 days, compared to RDV (unconjugated) which provided plasma levels of greater than 100 ng/ml for about 30 minutes.

RHa-5 showed an excellent sustained release profile for plasma Remdesivir, with the subcutaneous delivery providing a lower Cₘₐₓ and delayed Tₘₐₓ, and significantly increased Remdesivir bioavailability as compared to RDV (unconjugated). RHa-15 provided similar attributes, to a lesser degree.

All dendrimer-drug conjugates avoided the GS-441524 plasma spike shown by the reference product, and again, the sustained release pattern was evident. Cₘₐₓ and Tₘₐₓ were delayed for both conjugates, and in particular RHa-5, or the SC route. GS-441524 bioavailability following RHa-5 (SC) was slightly superior to that of the same conjugate delivered by the IV route.

Dendrimer-drug conjugates provided plasma levels of GS-441524 greater than 10 ng/ml for at least 3 days. In comparison, RDV (unconjugated) provided plasma levels of greater than 10 ng/ml for about 2 days. Remarkably, dendrimer-drug conjugates provided plasma levels of GS-441524 greater than 5 ng/ml for at least 7 days, compared to RDV (unconjugated) which provided plasma levels of greater than 5 ng/ml for about 3 days.

Taken together with the impressive Cₗₐₛₜ of RHa-15, these data indicate that dendrimer-drug conjugates can be used to provide an extended, 5 day, weekly or longer dose interval by either an IV or SC route of administration.

The AUC data for the important metabolite GS-441524, demonstrates it is available in equivalent quantities to RDV (unconjugated) delivered IV. As expected, AUCs of GS-441524 resulting from RDV (unconjugated) were the largest due to high plasma concentrations in the first hours. Otherwise, GS-441524 AUC are very similar between dendrimer constructs.

### REFERENCES

Chandel et al (2019) Biomedicine & Pharmaco doi: 10.1016/j.biopha.2019.108601 Coronaviridae Study Group of the International Committee on Taxonomy of Viruses (2020) Nature microbiology doi: 10.1038/s41564-020-0695-z.
Foster et al (2020) PNAS https://doi.org/10.1073/pnas.2004999117
Gordon et al (2020) bioRxiv doi: 10.1101/2020.03.22.002386.
Ibrahim et al (2015) Med Devices (Auckl) doi: 10.2147/MDER.S48888.
Kucharski et al (2020) Lancet https://doi.org/10.1016/S1473-3099(20)30144-4
Shen et al (2020) Clin Infect Dis. doi: 10.1093/cid/ciaa203.
Tang et al (2020) National Science Review https://doi.org/10.1093/nsr/nwaa036
Ugurel et al., Turk J Biol., 2020, 44(3): 157-167
Wang et al., J. Medical Virology, 2020, 92: 667-674
WO2012167309 (2012)

1. A dendrimer-drug conjugate comprising:
i) a core unit (C); and
ii) building units (BU), each building unit being a lysine residue or an analogue thereof;
wherein the core unit is covalently attached to at least two building units via amide linkages, each amide linkage being formed between a nitrogen atom present in the core unit and the carbon atom of an acyl group present in a building unit; and
wherein the dendrimer-drug conjugate has from three to six generations of building units; and
wherein building units of different generations are covalently attached to one another via amide linkages formed between a nitrogen atom present in one building unit and the carbon atom of an acyl group present in another building unit;
the dendrimer-drug conjugate further comprising:
iii) a plurality of first terminal groups (T1) attached to an outer building unit of the dendrimer, comprising a drug moiety comprising a Remdesivir nucleoside and a cleavable linker that provides for controlled release of the drug moiety; and
iv) a plurality of second terminal groups (T2) attached to an outer building unit of the dendrimer, comprising a hydrophilic polymeric group;
or a pharmaceutically acceptable salt thereof.

2. A dendrimer-drug conjugate as claimed in claim 1, wherein the dendrimer-drug conjugate is capable of releasing in vivo:

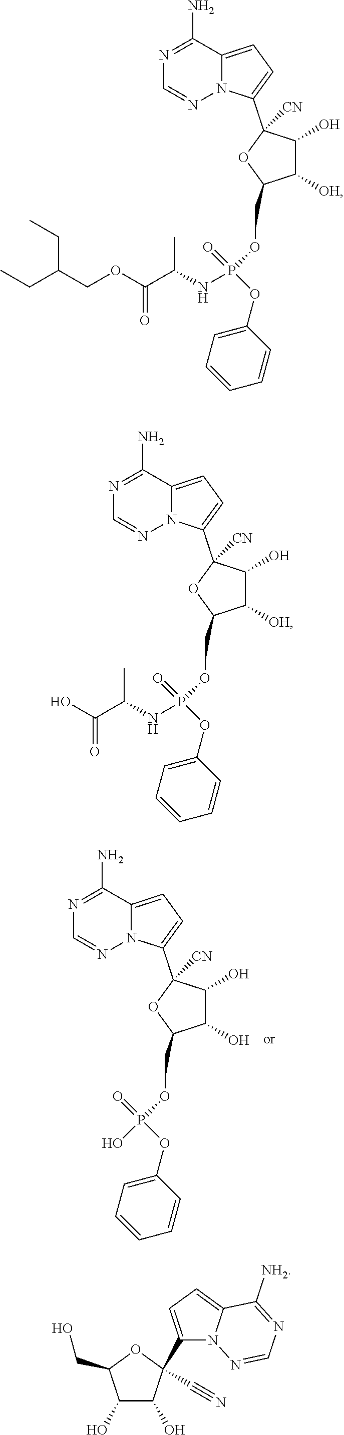

3. A dendrimer-drug conjugate as claimed in claim 2, wherein the dendrimer-drug conjugate is capable of releasing in vivo:

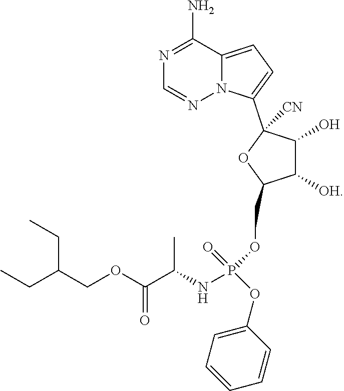

4. A dendrimer-drug conjugate as claimed in claim 2, wherein the dendrimer-drug conjugate is capable of releasing in vivo:

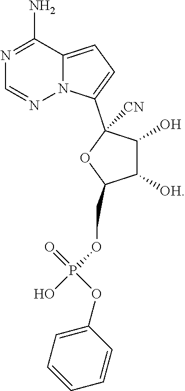

5. A dendrimer-drug conjugate as claimed in claim 2, wherein the dendrimer-drug conjugate is capable of releasing in vivo:

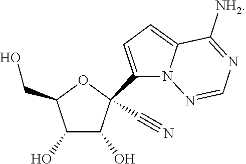 [SMILES: N#C[C@@]1(c2ccc3c(N)ncnn23)O[C@H](CO)[C@@H](O)[C@H]1O]

6. A dendrimer-drug conjugate as claimed in any of claims 1 to 5, wherein the core unit is formed from a core unit precursor comprising two amino groups.

7. A dendrimer-drug conjugate as claimed in any of claims 1 to 6, wherein the core unit is:

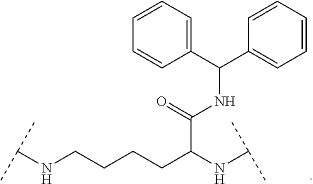

8. A dendrimer-drug conjugate as claimed in any of claims 1 to 7, wherein the building units are each: 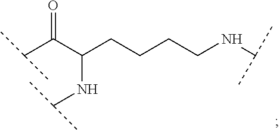
wherein the acyl group of each building unit provides a covalent attachment point for attachment to the core or to a previous generation building unit; and wherein each nitrogen atom provides a covalent attachment point for covalent attachment to a subsequent generation building unit, a first terminal group or a second terminal group.

9. A dendrimer-drug conjugate as claimed in claim 8, wherein the building units are each:

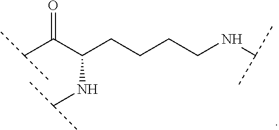

10. A dendrimer-drug conjugate as claimed in any of claims 1 to 9, wherein the dendrimer has five generations of building units.

11. A dendrimer-drug conjugate as claimed in any of claims 1 to 10, wherein the cleavable linker is covalently attached to the drug moiety such that, when exposed to PBS and 10% DMSO at pH 7.4 and 37° C., less than 50% of drug moiety is released from the conjugate within 24 hours.

12. A dendrimer-drug conjugate as claimed in any of claims 1 to 10, wherein the cleavable linker is covalently attached to the drug moiety such that, when exposed to PBS and 10% DMSO at pH 7.4 and 37° C., within 5% to 40% of drug moiety is released from the conjugate within 24 hours.

13. A dendrimer-drug conjugate as claimed in any of claims 1 to 12, wherein the cleavable linker is a diacyl linker group of formula: 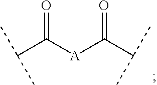
wherein A is a C₂-C₁₀ alkylene group which is optionally interrupted by at least one O, S, NH, or N(Me), or wherein A is a heterocycle selected from the group consisting of tetrahydrofuran, tetrahydrothiophene, pyrrolidine, and N-methylpyrrolidine.

14. A dendrimer-drug conjugate as claimed in any of claims 1 to 13, wherein the cleavable linker is:

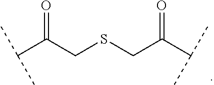

15. A dendrimer-drug conjugate as claimed in any of claims 1 to 14, wherein the drug moiety is: 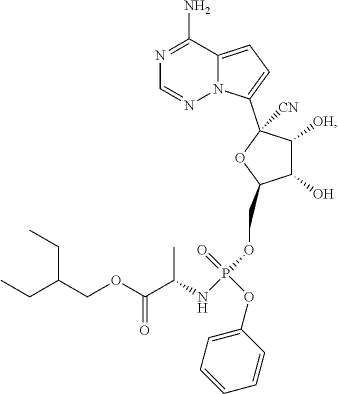
which is covalently attached to the cleavable linker through an —OH or —NH₂ group.

16. A dendrimer-drug conjugate as claimed in claim 15, wherein the drug moiety is selected from the group consisting of:

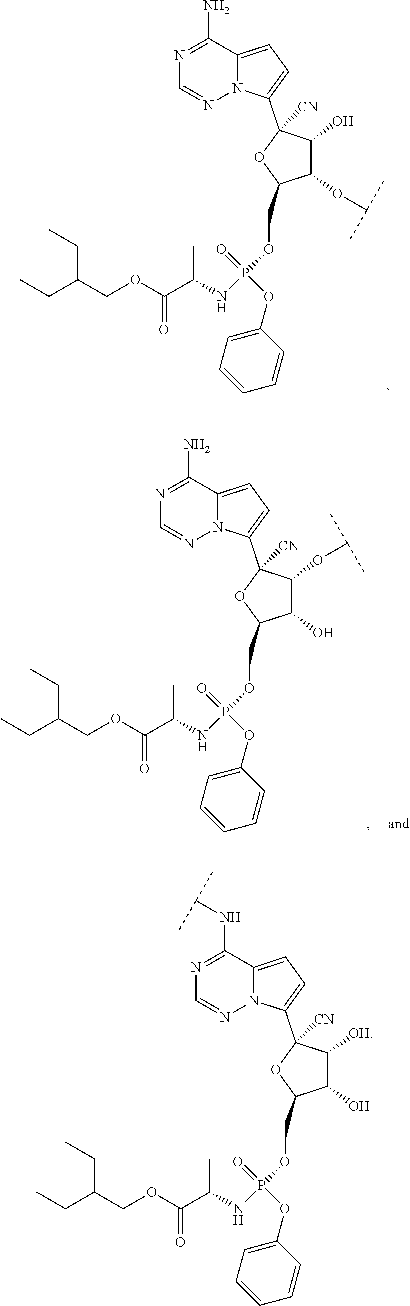

17. A dendrimer-drug conjugate as claimed in claim 16, wherein the first terminal group is:

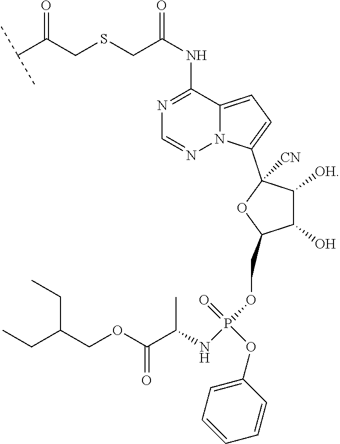

18. A dendrimer-drug conjugate as claimed in any of claims 1 to 14, wherein the drug moiety is selected from the group consisting of:

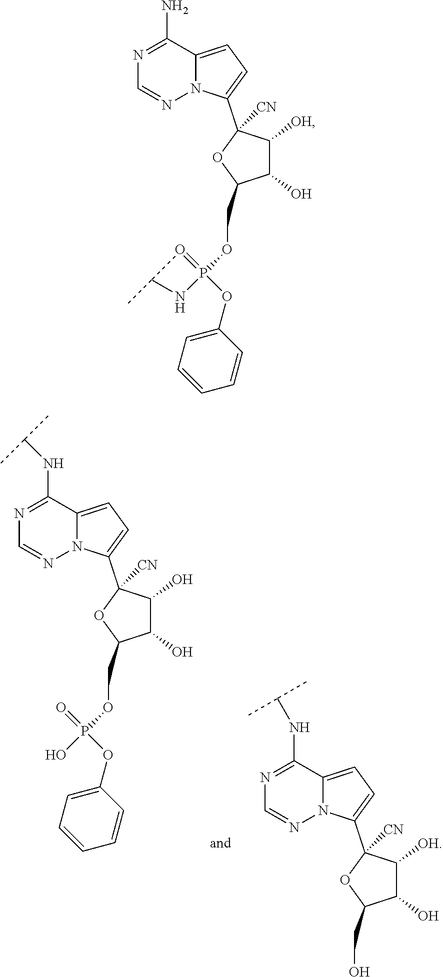

19. A dendrimer-drug conjugate as claimed in any of claims 1 to 18, wherein the hydrophilic polymers comprise polyethylene gycol (PEG), polyethyloxazoline (PEOX) or poly sarcosine groups.

20. A dendrimer-drug conjugate as claimed in claim 19, wherein the second terminal groups have an average molecular weight in the range of from 500 to 2500 Daltons.

21. A dendrimer-drug conjugate as claimed in claim 19 or 20, wherein the second terminal groups each comprise a PEG group covalently attached to a PEG linking group (L1) via an ether linkage formed between a carbon atom present in the PEG group and an oxygen atom present in the PEG linking group, and each second terminal group is covalently attached to a building unit via an amide linkage formed between a nitrogen atom present in a building unit and the carbon atom of an acyl group present in the PEG linking group.

22. A dendrimer-drug conjugate as claimed in claim 21, wherein the second terminal group is: 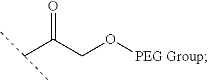
and wherein the PEG group is a methoxy-terminated PEG having an average molecular weight in the range of from 500 to 2500 Daltons.

23. A dendrimer-drug conjugate as claimed in claim 22, wherein the dendrimer-drug conjugate comprises surface units comprising an outer building unit attached to a first terminal group and a second terminal group, the surface units having the structure: 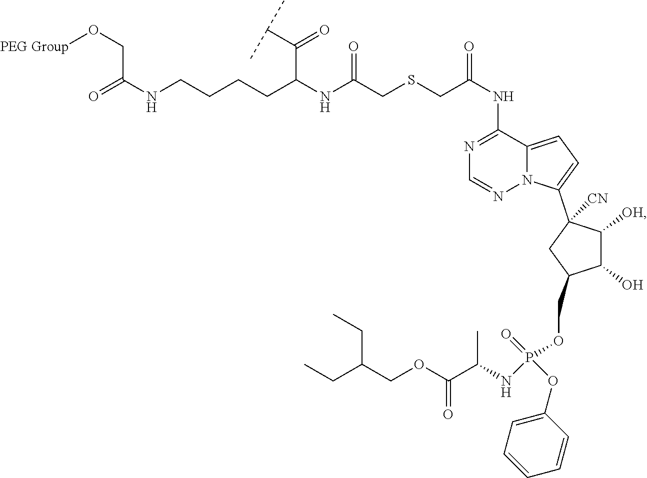
and wherein the PEG group is a methoxy-terminated PEG having an average molecular weight in the range of from 500 to 2500 Daltons.

24. A dendrimer-drug conjugate as claimed in any of claims 1 to 23, wherein the dendrimer has five generations of building units, the five generations are complete generations, and wherein the outer generation of building units provides 64 nitrogen atoms for covalent attachment to a first terminal group or a second terminal group, wherein from 24 to 32 first terminal groups are covalently attached to one of said nitrogen atoms, and wherein from 24 to 32 second terminal groups are each covalently attached to one of said nitrogen atoms.

25. A dendrimer-drug conjugate as claimed in claim 1, wherein the dendrimer-drug conjugate is: 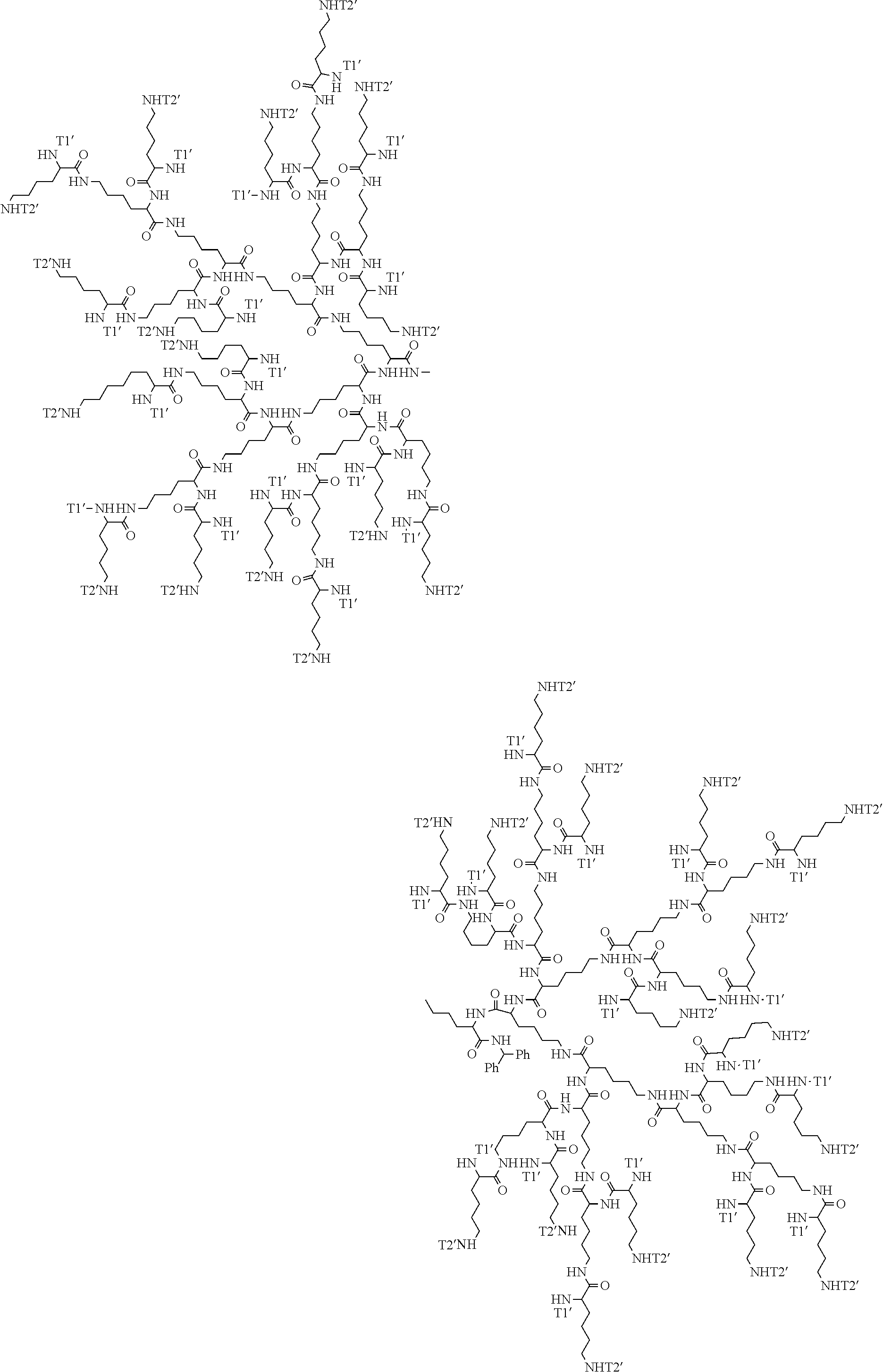
in which T1′ represents a group selected from the group consisting of hydrogen, and 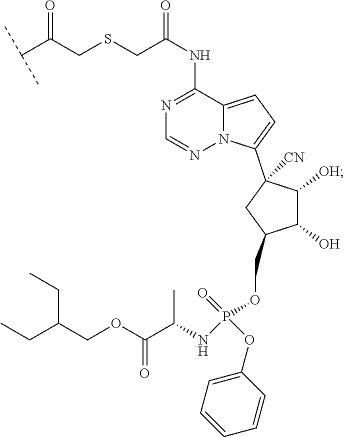
and wherein less than 10 of T1′ are hydrogen; and
T2′ represents a second group which is 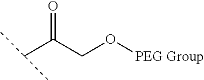
wherein the PEG group is a methoxy-terminated PEG having an average molecular weight in the range of from 500 to 2500 Daltons, or T2′ represents H, and wherein less than 10 of T2′ are H.

26. A composition comprising a plurality of dendrimer-drug conjugates or pharmaceutically acceptable salts thereof, wherein the dendrimer-drug conjugates are as defined in any of claims 1 to 25.

27. A pharmaceutical composition comprising:
i) a dendrimer-drug conjugate as claimed in any of claims 1 to 25, or a pharmaceutically acceptable salt thereof; and
ii) a pharmaceutically acceptable excipient.

28. A pharmaceutical composition as claimed in claim 27, wherein the composition is free of cyclodextrin.

29. A pharmaceutical composition as claimed in claim 27 or claim 28, wherein the composition has greater aqueous solubility of drug moiety comprising Remdesivir nucleoside than Remdesivir, in terms of moles of Remdesivir nucleoside solubilised.

30. A pharmaceutical composition as claimed in claim 27 or claim 28, wherein the composition is a non-aqueous composition formulated for intramuscular injection.

31. A pharmaceutical composition as claimed in claim 27 or claim 28, wherein the composition is a solid composition formulated for pulmonary delivery.

32. A pharmaceutical composition as claimed in claim 27 or claim 28, wherein the composition is formulated for pulmonary delivery.

33. A dendrimer-drug conjugate as claimed in any of claims 1 to 25, or a pharmaceutical composition as claimed in any of claims 27 to 32, for use in the treatment and/or prevention of a viral infection.

34. A method of treating and/or preventing a viral infection comprising administering to a subject in need thereof a therapeutically effective amount of a dendrimer-drug conjugate as claimed in any of claims 1 to 25, or a pharmaceutical composition according to claims 27 to 32.

35. Use of a dendrimer-drug conjugate as claimed in any of claims 1 to 25, or of a composition as claimed in any of claims 27 to 32, in the manufacture of a medicament for the treatment and/or prevention of a viral infection.

36. A method, use, or dendrimer-drug conjugate or composition for use, as claimed in any of claims 33 to 35, wherein the viral infection is an RNA viral infection.

37. A method, use, or dendrimer-drug conjugate or composition for use, as claimed in any of claims 33 to 36, wherein the viral infection is a Coronavirus (CoV) infection.

38. A method, use, or dendrimer-drug conjugate or composition for use, as claimed in claim 37, wherein the Coronavirus (CoV) is selected from the group consisting of severe acute respiratory syndrome-related coronavirus-2 (SARS-CoV-2), human coronavirus OC43 (HCoV-OC43), human coronavirus HKU1 (HCoV-HKU1), human coronavirus 229E (HCoV-229E), human coronavirus NL63 (HCoV-NL63), severe acute respiratory-related coronavirus (SARS-CoV), and middle-east respiratory syndrome-related coronavirus (MERS-CoV), and subtypes or variants thereof.

39. A method, use, or dendrimer-drug conjugate or composition for use, as claimed in claim 38, wherein the Coronavirus (CoV) is SARS-CoV-2 or a subtype or variant thereof.

40. A method, use, or dendrimer-drug conjugate or composition for use, as claimed in any of claims 37 to 39, wherein the prevention and/or treatment of a viral infection includes preventing or reducing the likelihood or severity of a symptom associated with a Coronavirus (CoV) infection.

41. A method, use, or dendrimer-drug conjugate or composition for use, as claimed in claim 40, wherein the symptom associated with a Coronavirus (CoV) infection is one or more selected from the group consisting of fever, cough, sore throat, shortness of breath, viral shedding, respiratory insufficiency, runny nose, nasal congestion, bronchitis, headache, muscle pain, dyspnea, moderate pneumonia, severe pneumonia, and acute respiratory distress syndrome (ARDS).

42. A method, use, or dendrimer-drug conjugate or composition for use as claimed in any of claims 33 to 41, wherein the dendrimer-drug conjugate or composition is administered parenterally.

43. A method, use, or dendrimer-drug conjugate or composition for use as claimed in any of claims 33 to 42, wherein the dendrimer-drug conjugate or composition is administered intravenously.

44. A method, use, or dendrimer-drug conjugate or composition for use as claimed in any of claims 33 to 43, wherein the dendrimer-drug conjugate or composition is administered by fast infusion or as a bolus.

45. A method, use, or dendrimer-drug conjugate or composition for use as claimed in any of claims 33 to 42, wherein the dendrimer-drug conjugate or composition is administered intramuscularly.

46. A method, use, dendrimer-drug conjugate or composition for use as claimed in any of claims 33 to 42, wherein the dendrimer-drug conjugate or composition is administered subcutaneously.

47. A method, use, dendrimer-drug conjugate or composition for use as claimed in any of claims 33 to 41, wherein the dendrimer-drug conjugate or composition is administered by inhalation.

48. A method, use, or dendrimer-drug conjugate or composition for use, as claimed in any of claims 33 to 47, wherein a single dose of dendrimer-drug conjugate provides plasma levels of Remdesivir of greater than 10 ng/mL for at least 5 days.

49. A method, use, or dendrimer-drug conjugate or composition for use, as claimed in any of claims 33 to 48, wherein a single dose of dendrimer-drug conjugate provides plasma levels of Remdesivir of greater than 100 ng/mL for at least 2 days.

50. A method, use, or dendrimer-drug conjugate or composition for use, as claimed in any of claims 33 to 49, wherein a single dose of dendrimer-drug conjugate provides plasma levels of GS-441524 of greater than 10 ng/mL for at least 2 days.

51. A method, use, or dendrimer-drug conjugate or composition for use, as claimed in any of claims 33 to 45, wherein a single dose of dendrimer-drug conjugate provides plasma levels of GS-441524 of greater than 5 ng/mL for at least 5 days.

52. A method, use, or dendrimer-drug conjugate or composition for use, as claimed in any of claims 33 to 47, wherein a single dose of dendrimer-drug conjugate provides a therapeutically effective amount of Remdesivir nucleoside over a period of at least five days.

53. A method, use, or dendrimer-drug conjugate or composition for use, as claimed in any of claims 33 to 52, wherein a single dose of dendrimer-drug conjugate provides a therapeutically effective amount of the drug moiety comprising Remdesivir nucleoside over a period of at least two days.

54. A method, use, or dendrimer-drug conjugate or composition for use, as claimed in any of claims 33 to 53, wherein a single dose of dendrimer-drug conjugate provides therapeutic drug exposure (AUCinf) of at least 5000 ng/h/mL of Remdesivir.

55. A method, use, or dendrimer-drug conjugate or composition for use, as claimed in any of claims 33 to 54, wherein a single dose of dendrimer-drug conjugate provides therapeutic drug exposure (AUCinf) of at least 3000 ng/h/mL of GS-441524.

56. A method, use, or dendrimer-drug conjugate or composition for use as claimed in any of claims 33 to 55, wherein the dendrimer is administered in combination with a further therapeutic agent used for therapy of a viral condition.
